(19) United States
(12) Patent Application Publication
(10) Pub. No.: US 2009/0069408 A1
(43) Pub. Date: Mar. 12, 2009

(54) TRICYCLIC COMPOUNDS USEFUL IN TREATING IRON DISORDERS

(75) Inventors: Mikhail Chafeev, Burnaby (CA); Nagasree Chakka, Burnaby (CA); Jean-Jacques Cadieux, Burnaby (CA); Jianmin Fu, Coquitlam (CA); Rajender Kamboj, Burnaby (CA); Vishnumurthy Kodumuru, Burnaby (CA); Jonathan Langille, Langley (CA); Shifeng Liu, Port Coquitlam (CA); Jianyu Sun, Richmond (CA); Serguei Sviridov, Burnaby (CA); Zaihui Zhang, Vancouver (CA)

Correspondence Address: Seed IP Law Group PLLC, 701 Fifth Avenue, Suite 5400, Seattle, WA, 98104, US

(73) Assignee: XENON PHARMACEUTICALS INC., Burnaby (CA)

(21) Appl. No.: 12/044,731

(22) Filed: Mar. 7, 2008

Related U.S. Application Data
(60) Provisional application No. 60/893,585, filed on Mar. 7, 2007.

(51) Int. Cl. A61K 31/381; C07D 333/76; A61K 31/343; C07D 307/91; C07C 257/10; A61K 31/155

(52) U.S. Cl. 514/443; 549/43; 514/468; 549/460; 564/243; 514/636

(57) ABSTRACT

This invention is directed to, for example, compounds of formula (I):

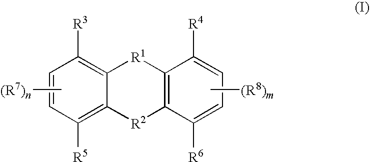

wherein n, m, R¹, R², R³, R⁴, R⁵, R⁶, R⁷ and R⁸ are as defined herein, as a stereoisomer, enantiomer, tautomer thereof or mixtures thereof; or a pharmaceutically acceptable salt, solvate or prodrug thereof, for the treatment of iron disorders. This invention is also directed to pharmaceutical compositions comprising the compounds and methods of using the compounds to treat iron disorders.

### CROSS-REFERENCE TO RELATED APPLICATION

This application claims the benefit under 37 U.S.C. § 119(e) of U.S. Provisional Patent Application No. 60/893,585 filed Mar. 7, 2007, which application is incorporated herein by reference in its entirety.

### FIELD OF THE INVENTION

The present invention is directed to tricyclic compounds which are divalent metal transporter-1 inhibitors. The compounds of the invention, and pharmaceutical compositions comprising the compounds, are therefore useful in treating iron disorders in mammals.

### BACKGROUND OF THE INVENTION

Iron is an essential metal for life because it is a key constituent of a family of fundamental proteins, which includes hemoglobin, cytochromes, and NADH-coenzyme Q reductase. Maintaining body iron homeostasis is paramount to health because iron deficiency or excess results in morbidity and mortality.

Divalent metal transporter-1 (DMT1), also known as natural resistance-associated macrophage protein-2 (NRAMP2) and divalent cation transporter-1 (DCT1), is a ubiquitiously expressed transmembrane protein involved in the maintenance of iron levels in the body. DMT1 is particularly important for iron absorption in the duodenum of the small intestine, where it is localized in the cytoplasm and brush border membrane of the villus enterocytes and mediates the influx of dietary non-heme iron from the intestinal lumen into the enterocytes (Gunshin et al., J. Clin. Invest., 2005, 115:1258-1266). Once dietary iron is absorbed across the intestinal wall, there is no physiologic mechanism for excreting iron from the body. Thus, excess absorbed iron is largely retained in the body and can accumulate throughout life. Excess accumulation of iron leads to considerable tissue damage and increased subsequent disease risk such as, for example, cirrhosis or hepatocellular carcinoma. Therefore, DMT1 is the primary focal point of controlling intestinal iron absorption for the maintenance of body iron homeostatsis.

There is compelling evidence to support that DMT1 activity is tightly associated with many common diseases, such as, but not limited to, primary iron overload disorders, especially diseases related to hereditary hemochromatosis (Rolfs et al., Am. J. Physiol. Gastrointest Liver Physiol., 2002, 282(4):G598-607). Further, DMT1 plays a significant role in intestinal iron hyperabsorption in patients suffering from hypochromic microcytic anemias and related disorders (Morgan et al., Blood Cell, Molecules, and Diseases, 2002, 29(3):384-399).

To date, there are only three known small-molecule, drug-like compounds that specifically modulate or inhibit DMT1 (Welti et al., Chem. Biol., 2006, 13:965-972). Accordingly, there is an unmet medical need to treat iron disorders, preferably primary iron overload and transfusional iron overload, including thalassemia, in mammals, preferably in humans, effectively and without adverse side effects. The present invention provides compounds and methods to meet these critical needs.

### SUMMARY OF THE INVENTION

The present invention is directed to tricyclic compounds of the invention and pharmaceutical compositions comprising the compounds for the treatment of iron disorders.

Accordingly, in one aspect this invention provides compounds of formula (I):

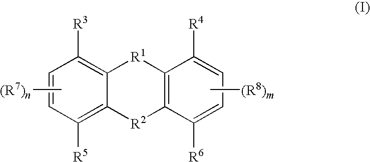

wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are different and are each independently selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³, or —R¹¹—N(R⁹)—C(═NR¹²)N(R¹²)R¹³; or R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³, or —R¹¹—N(R⁹)—C(═NR¹²)N(R¹²)R¹³;

R⁵ and R⁶ are different and are each independently selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
or R⁵ and R⁶ are the same and are selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴) —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³—N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)R¹⁴, —R¹¹—C(O)R¹⁴, —R¹¹—C(S)R¹⁵, —R¹¹—C(O)OR¹⁴, —R¹¹—OC(O)R¹⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl, or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl;
   as a stereoisomer, enantiomer, tautomer thereof or mixtures thereof;
   or a pharmaceutically acceptable salt, solvate or prodrug thereof.

In another aspect, the invention provides pharmaceutical compositions comprising a pharmaceutically acceptable excipient and a compound of formula (I), as a stereoisomer, enantiomer, tautomer thereof or mixtures thereof, or as a pharmaceutically acceptable salt, solvate or prodrug thereof.

In another aspect, this invention provides compounds of formula (II):

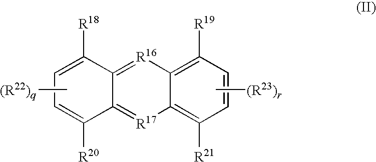

wherein:
q and r are each independently 0, 1 or 2;
R¹⁶ and R¹⁷ are each independently ═C(R²⁴)— or ═N—;
R¹⁸ and R¹⁹ are different and are each independently selected from —R²⁵ SC(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
or R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(—NR²⁶)N(R²⁶)R²⁷ or —R²⁵—N(R⁹)—C(═NR²⁶)N(R²⁶)R²⁷;
R²⁰ and R²¹ are different and are each independently selected from hydrogen, alkyl, halo, haloalkyl, —R²⁵—CN, —R²⁵—NO₂, —R²—N(R)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
or R²⁰ and R²¹ are the same and are selected from hydrogen, alkyl, halo, haloalkyl, —R²⁵CN, —R²⁵—NO₂, —R²⁵—N(R²⁸)₂, —R²⁵—C(O)R²⁸, —R²⁵—C(ON(R²⁸)₂, —R²⁵═SC(NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)₂, —R²⁵—S(O)R²⁸, —R²⁵—C(O)R²⁸, —R²⁵—C(S)R²⁹, —R²⁵—C(O)R²⁸, —R²⁵—OC(O)R²⁸, —R²⁵—C(S)OR²⁸, —R²⁵—C(O)N(R²⁸)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl;
   as a stereoisomer, enantiomer, tautomer thereof or mixtures thereof;
   or a pharmaceutically acceptable salt, solvate or prodrug thereof.

In another aspect, the invention provides pharmaceutical compositions comprising a pharmaceutically acceptable excipient and a compound of formula (II), as a stereoisomer, enantiomer, tautomer thereof or mixtures thereof, or as a pharmaceutically acceptable salt, solvate or prodrug thereof.

In another aspect, the invention provides methods for treating an iron disorder in a mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods for treating a disease or condition associated with an iron disorder in a mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods for treating a disease or condition associated with an iron disorder in a mammal due to accumulation of iron in the body tissues of the mammal, wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods for treating an iron disorder in a mammal or a disease or condition associated with an iron disorder in a mammal, wherein the iron disorder, disease or condition is associated with increased DMT1 activity and wherein the methods comprise administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides methods of inhibiting the activity of DMT1 in a cell, preferably a mammalian cell, wherein the methods comprise contacting the mammalian cell with a DMT1-inhibitory amount of a compound of the invention, as set forth above, as a stereoisomer, enantiomer, tautomer thereof or mixtures thereof, or a pharmaceutically acceptable salt, solvate or prodrug thereof.

In another aspect, the invention provides methods of treating an iron disorder in a mammal, wherein the iron disorder is ameliorated by the inhibition of the activity of DMT1 in the mammal and wherein the methods comprise administering to the mammal a DMT1-inhibiting amount of a compound of the invention, as set forth above, as a stereoisomer, enantiomer, tautomer thereof or mixtures thereof, or a pharmaceutically acceptable salt, solvate or prodrug thereof, or a DMT1-inhibiting amount of a pharmaceutical composition comprising a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

In another aspect, the invention provides pharmaceutical therapy in combination with one or more other compounds of the invention or one or more other accepted therapies or as any combination thereof to increase the potency of an existing or future drug therapy or to decrease the adverse events associated with the accepted therapy.

In one embodiment, the invention relates to a pharmaceutical composition combining compounds of the present invention with established or future therapies for the indications listed in the invention.

In another aspect, this invention is directed to the use of the compounds of the invention, as set forth above, as a stereoisomer, enantiomer, tautomer thereof or mixtures thereof, or a pharmaceutically acceptable salt, solvate or prodrug thereof, or the use of a pharmaceutical composition comprising a pharmaceutically acceptable excipient and a compound of the invention, as set forth above, as a stereoisomer, enantiomer, tautomer thereof or mixtures thereof, or a pharmaceutically acceptable salt, solvate or prodrug thereof, in the preparation of a medicament for the treatment of iron disorders in a mammal.

### DETAILED DESCRIPTION OF THE INVENTION

### Definitions

Certain chemical groups named herein may be preceded by a shorthand notation indicating the total number of carbon atoms that are to be found in the indicated chemical group. For example; C₇-C₁₂alkyl describes an alkyl group, as defined below, having a total of 7 to 12 carbon atoms, and C₄-C₁₂cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, as used in the specification and appended claims, unless specified to the contrary, the following terms have the meaning indicated:

“Amino” refers to the —NH₂ radical.

“Cyano” refers to the —CN radical.

“Hydroxy” refers to the —OH radical.

“Imino” refers to the ═NH substituent.

“Nitro” refers to the —NO₂ radical.

“Oxo” refers to the ═O substituent.

“Thioxo” refers to the ═S substituent.

“Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, preferably one to eight carbon atoms or one to six carbon atoms, and which is attached to the rest of the molecule by a single bond, e.g., methyl, ethyl, n-propyl, 1-methylethyl(iso-propyl), n-butyl, n-pentyl, 1,1-dimethylethyl(t-butyl), 3-methylhexyl, 2-methylhexyl, and the like. Unless stated otherwise specifically in the specification, an alkyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR³⁰, —OC(O)—R³⁰, —N(R³⁰)₂, —C(O)R³⁰, —C(O)OR³⁰, —C(O)N(R³⁰)₂, —N(R³⁰)C(O)OR³², —N(R³)C(O)R³², —N(R³⁰)S(O)ₜR³² (where t is 1 to 2), —S(O)ₜOR³² (where t is 1 to 2), —S(O)ₚR³² (where p is 0 to 2), and —S(O)ₜN(R³⁰)₂ (where t is 1 to 2) where each R³⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R³² is alkyl, haloalkyl, cycloalkyl, cycloalkylalkyl, aryl, aralkyl, heterocyclyl, heterocyclylalkyl, heteroaryl or heteroarylalkyl.

“Alkenyl” refers to a straight or branched hydrocarbon chain radical group consisting solely of carbon and hydrogen atoms, containing at least one double bond, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR³⁰, —OC(O)—R³⁰, —N(R³⁰)₂, —C(O)R³⁰, —C(O)OR³⁰, —C(O)N(R³⁰)₂, —N(R³⁰)C(O)OR³², —N(R³⁰)C(O)R³², —N(R³⁰)S(O)ₜR³² (where t is 1 to 2), —S(O)ₜOR³² (where t is 1 to 2), —S(O)ₚR³² (where p is 0 to 2), and —S(O)ₜN(R³⁰)₂ (where t is 1 to 2) where each R³⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R³² is alkyl, haloalkyl, cycloalkyl, cycloalkylalkyl, aryl, aralkyl, heterocyclyl, heterocyclylalkyl, heteroaryl or heteroarylalkyl.

“Alkynyl” refers to a straight or branched hydrocarbon chain radical group comprising solely of carbon and hydrogen atoms, containing at least one triple bond, optionally containing at least one double bond, having from two to twelve carbon atoms, preferably two to eight carbon atoms and which is attached to the rest of the molecule by a single bond, for example, ethynyl, propynyl, butynyl, pentynyl, hexynyl, and the like. Unless stated otherwise specifically in the specification, an alkynyl group may be optionally substituted by one or more of the following substituents: alkyl, alkenyl, halo, haloalkenyl, cyano, nitro, aryl, cycloalkyl, heterocyclyl, heteroaryl, oxo, trimethylsilanyl, —OR³⁰, —OC(O)—R³⁰, —N(R³⁰)₂, —C(O)R³⁰, —C(O)OR³⁰, —C(O)N(R³⁰)₂, —N(R³⁰)C(O)OR³², —N(R³⁰)C(O)R³², —N(R³⁰)S(O)ₜR³² (where t is 1 to 2), —S(O)ₜOR³² (where t is 1 to 2), —S(O)ₚR³² (where p is 0 to 2), and —S(O)ₜN(R³)₂ (where t is 1 to 2) where each R³⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R³² is alkyl, haloalkyl, cycloalkyl, cycloalkylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR³⁰, —OC(O)—R³⁰, —N(R³)₂, —C(O)R³⁰, —C(O)OR³⁰—C(O)N(R³⁰)₂—N(R³⁰)C(O)OR³², —N(R³⁰)C(O)R³², —N(R³⁰)S(O)ₜR³² (where t is 1 to 2), —S(O)ₜOR³² (where t is 1 to 2), —S(O)ₚR³² (where p is 0 to 2), and —S(O)ₜN(R³⁰)₂ (where t is 1 to 2) where each R³⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R³² is alkyl, haloalkyl, cycloalkyl, cycloalkylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR³⁰, —OC(O)—R³⁰, —N(R³⁰)₂, —C(O)R³⁰, —C(O)OR³⁰, —C(O)N(R³⁰)₂, —N(R³⁰)C(O)OR³², —N(R³⁰)C(O)R³², N(R³⁰)S(O)ₜR³² (where t is 1 to 2), —S(O)ₜOR³² (where t is 1 to 2), —S(O)ₚR³² (where p is 0 to 2), and —S(O)ₜN(R³⁰)₂ (where t is 1 to 2) where each R³⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R³² is alkyl, haloalkyl, cycloalkyl, cycloalkylalkyl, aryl, aralkyl, heterocyclyl, heterocyclylalkyl, heteroaryl or heteroarylalkyl.

“Alkoxy” refers to a radical of the formula —ORₐ where Rₐ is an alkyl radical as defined above containing one to twelve carbon atoms. The alkyl part of the alkoxy radical may be optionally substituted as defined above for an alkyl radical.

“Alkoxyalkyl” refers to a radical of the formula —R_{b}—O—Rₐ where R_{b} is an alkylene chain as defined above and R^{a} is an alkyl radical as defined above. The oxygen atom may be bonded to any carbon in the alkylene chain and in the alkyl radical. The alkyl part of the alkoxyalkyl radical may be optionally substituted as defined above for an alkyl group. The alkylene chain part of the alkoxyalkyl radical may be optionally substituted as defined above for an alkylene chain.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may included fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals optionally substituted by one or more substituents independently selected from the group consisting of alkyl, akenyl, halo, haloalkyl, haloalkenyl, cyano, nitro, aryl, aralkyl, heteroaryl, heteroarylalkyl, —R³¹—OR³⁰, —R³¹—OC(O)—R³⁰, —R³¹—N(R³⁰)₂, —R³¹—C(O)R³⁰, —R³¹—C(O)OR³⁰, —R³¹—C(O)N(R³⁰)₂, —R³¹—N(R³⁰)C(O)OR³², —R³¹—N(R³⁰)C(O)R³², —R³¹—N(R³⁰)S(O)ₜR³² (where t is 1 to 2), —R³¹—N═C(OR³⁰)R³⁰, —R³¹—S(O)ₜOR³² (where t is 1 to 2), —R³¹—S(O)ₚR³² (where p is 0 to 2), and —R³¹—S(O)ₜN(R³⁰)₂ (where t is 1 to 2) where each R³⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; each R³¹ is independently a direct bond or a straight or branched alkylene or alkenylene chain; and each R³² is alkyl, haloalkyl, cycloalkyl, cycloalkylalkyl, aryl, aralkyl, heterocyclyl, heterocyclylalkyl, heteroaryl or heteroarylalkyl.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. The alkylene chain part of the aralkyl radical may be optionally substituted as described above for an alkylene chain. The aryl part of the aralkyl radical may be optionally substituted as described above for an aryl group.

“Aralkenyl” refers to a radical of the formula —R^{d}—R_{c} where R_{d} is an alkenylene chain as defined above and R_{c} is one or more aryl radicals as defined above. The aryl part of the aralkenyl radical may be optionally substituted as described above for an aryl group. The alkenylene chain part of the aralkenyl radical may be optionally substituted as defined above for an alkenylene group.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, the term “cycloalkyl” is meant to include cycloalkyl radicals which are optionally substituted by one or more substituents independently selected from the group consisting of alkyl, alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R³¹—OR³⁰, —R³¹—OC(O)—R³⁰, —R³¹—N(R³⁰) —R³¹—C(O)R³⁰, —R³¹—C(O)OR³⁰, —R³¹—C(O)N(R³⁰)₂, —R³¹—N(R³⁰)C(O)OR³², —R³¹—N(R³⁰)C(O)R³², —R³¹—N(R³⁰)S(O)ₜR³² (where t is 1 to 2), —R³¹—N═C(OR³⁰)R³⁰, —R³¹—S(O)ₜOR³² (where t is 1 to 2), —R³¹—S(O)ₚR³² (where p is 0 to 2), and —R³¹—S(O)ₜN(R³⁰)₂ (where t is 1 to 2) where each R³⁰ is independently hydrogen, alkyl, haloalkyl, cycloalkyl, cycloalkylalkyl, aryl, aralkyl, heterocyclyl, heterocyclylalkyl, heteroaryl or heteroarylalkyl; each R³¹ is independently a direct bond or a straight or branched alkylene or alkenylene chain; and each R³² is alkyl, haloalkyl, cycloalkyl, cycloalkylalkyl, aryl, aralkyl, heterocyclyl, heterocyclylalkyl, heteroaryl or heteroarylalkyl.

“Cycloalkylalkyl” refers to a radical of the formula —R_{b}R_{g} where R_{b} is an alkylene chain as defined above and R_{g} is a cycloalkyl radical as defined above. The alkylene chain and the cycloalkyl radical may be optionally substituted as defined above.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Haloalkenyl” refers to an alkenyl radical, as defined above, that is substituted by one or more halo radicals, as defined above. The alkenyl part of the haloalkyl radical may be optionally substituted as defined above for an alkenyl group.

“Heterocyclyl” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above which are optionally substituted by one or more substituents selected from the group consisting of alkyl, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R³¹—OR³⁰, —R³¹—OC(O)—R³⁰, —R³¹—N(R³⁰)₂, —R³¹—C(O)R³⁰, —R³¹—C(O)OR³⁰, —R³—C(O)N(R³⁰)₂—R³¹—N(R³⁰)C(O)OR³², —R³¹—N(R³⁰)C(O)R³², —R³¹—N(R³⁰)S(O)ₜR³² (where t is 1 to 2), —R³¹—N═C(OR³⁰)R³⁰, —R³¹—S(O)ₜOR³² (where t is 1 to 2), —R³¹—S(O)ₚR³² (where p is 0 to 2), and —R³¹—S(O)ₜN(R³⁰)₂ (where t is 1 to 2) where each R³⁰ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R³¹ is independently a direct bond or a straight or branched alkylene or alkenylene chain; and each R³² is alkyl, alkenyl, haloalkyl, cycloalkyl, cycloalkylalkyl, aryl, aralkyl, heterocyclyl, heterocyclylalkyl, heteroaryl or heteroarylalkyl.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical may be optionally substituted as described above for heterocyclyl radicals.

“Heterocyclylalkyl” refers to a radical of the formula —R_{b}Rₕ where R_{b} is an alkylene chain as defined above and Rₕ is a heterocyclyl radical as defined above, and if the heterocyclyl is a nitrogen-containing heterocyclyl, the heterocyclyl may be attached to the alkyl radical at the nitrogen atom. The alkylene chain of the heterocyclylalkyl radical may be optionally substituted as defined above for an alkyene chain. The heterocyclyl part of the heterocyclylalkyl radical may be optionally substituted as defined above for a heterocyclyl group.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, the term “heteroaryl” is meant to include heteroaryl radicals as defined above which are optionally substituted by one or more substituents selected from the group consisting of alkyl, alkenyl, alkoxy, halo, haloalkyl, haloalkenyl, cyano, oxo, nitro, thioxo, aryl, aralkyl, cycloalkyl, cycloalkylalkyl, heterocyclyl, heterocyclylalkyl, heteroaryl, heteroarylalkyl, —R¹³—OR³⁰, —R³¹—OC(O)—R³⁰, —R³¹—N(R³⁰)₂, —R³¹—C(O)R³⁰, —R³¹—C(O)OR³⁰, —R³¹—C(O)N(R³⁰)₂, —R³¹—N(R³⁰)C(O)OR³², —R³¹—N(R³⁰)C(O)R³², —R³¹—N(R³⁰)S(O)ₜR³² (where t is 1 to 2), —R³¹—N═C(OR³⁰)R³⁰, —R³¹—S(O)ₜOR³² (where t is 1 to 2), —R³¹—S(O)ₚR³² (where p is 0 to 2), and —R³¹—S(O)ₜN(R³⁰)₂ (where t is 1 to 2) where each R³⁰ is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R³¹ is independently a direct bond or a straight or branched alkylene or alkenylene chain; and each R³² is alkyl, alkenyl, haloalkyl, cycloalkyl, cycloalkylalkyl, aryl, aralkyl, heterocyclyl, heterocyclylalkyl, heteroaryl or heteroarylalkyl.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical may be optionally substituted as described above for heteroaryl radicals.

“Heteroarylalkyl” refers to a radical of the formula —R_{b}Rᵢ where R_{b} is an alkylene chain as defined above and Rᵢ is a heteroaryl radical as defined above. The heteroaryl part of the heteroarylalkyl radical may be optionally substituted as defined above for a heteroaryl group. The alkylene chain part of the heteroarylalkyl radical may be optionally substituted as defined above for an alkylene chain.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of formula (I) and formula (II) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the binding affinity to pharmacologically important site of action on DMT1. Certain isotopically-labelled compounds of formula (I) and formula (II), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) and formula (II) can generally be prepared by conventional techniques known to those skilled in the art or by processes analogous to those described in the Preparations and Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reducation, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its coversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets, (e.g. cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. When a functional group is described as “optionally substituted,” and in turn, substitutents on the functional group are also “optionally substituted” and so on, for the purposes of this invention, such iterations are limited to five, preferably such iterations are limited to two.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Therapeutically effective amount” refers to that amount of a compound of the invention which, when administered to a mammal, preferably a human, is sufficient to effect treatment, as defined below, of an iron disorder or a disease or condition associated with an iron disorder, in the mammal, preferably a human. The amount of a compound of the invention which constitutes a “therapeutically effective amount” will vary depending on the compound, the iron disorder, disease or condition and its severity, the manner of administration, and the age of the mammal to be treated, but can be determined routinely by one of ordinary skill in the art having regard to his own knowledge and to this disclosure. Preferably, for purposes of this invention, a “therapeutically effective amount” is that amount of a compound of invention which is sufficient to inhibit the activity of DMT1.

“Treating” or “treatment”, as used herein, covers the treatment of an iron disorder in a mammal, preferably a human, or a disease or condition associated with an iron disorder in a mammal, preferably a human, and includes:

(i) preventing an iron disorder in a mammal, or a disease or condition associated with an iron disorder in the mammal, from occurring in the mammal;

(ii) inhibiting an iron disorder in a mammal, or a disease or condition associated with an iron disorder in the mammal, i.e., arresting its development;

(iii) relieving an iron disorder in a mammal, or a disease or condition associated with an iron disorder in the mammal, i.e., causing regression of the iron disorder or the disease or condition;

(iv) relieving the symptoms of an iron disorder in a mammal, or a disease or condition associated with an iron disorder in the mammal, i.e., relieving the symptoms without addressing the underlying iron disorder, disease or condition; or

(v) restoring and/or maintaining normal serum iron levels, transferrin saturation, serum ferritin, liver iron and/or bodily iron levels in a mammal having an iron disorder or having a disease or condition associated with an iron disorder.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)-for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

Also within the scope of the invention are intermediate compounds of formula (I) and formula (II) and all polymorphs of the aforementioned species and crystal habits thereof.

The chemical naming protocol and structure diagrams used herein are a modified form of the I.U.P.A.C. nomenclature system, using the ChemDraw Version 10 software program (CambridgeSoft®), wherein the compounds of the invention are named herein as derivatives of the central core structure, i.e., the tricyclic structure. For complex chemical names employed herein, a substituent group is named before the group to which it attaches. For example, cyclopropylethyl comprises an ethyl backbone with cyclopropyl substituent. In chemical structure diagrams, all bonds are identified, except for some carbon atoms, which are assumed to be bonded to sufficient hydrogen atoms to complete the valency.

Thus, for example, a compound of formula (I) wherein n and m are both 0, R¹ is —S—, R² is a direct bond, R³ and R⁴ are both —CH₂—S—C(═NH)NH₂, and R⁵ and R⁶ are both hydrogen; i.e., a compound of the following formula:

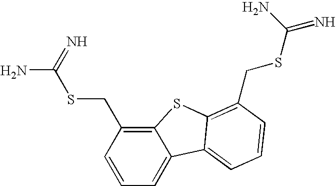

is named herein as dibenzo[b,d]thiophene-4,6-diylbis(methylene) dicarbamimidothioate.

### EMBODIMENTS OF THE INVENTION

Of the various aspects of the invention set forth above in the Summary of the Invention, certain embodiments of the aspects are preferred.

One aspect of the invention is a compound of formula (I), as set forth above in the Summary of the Invention, as a stereoisomer, enantiomer, tautomer thereof or mixtures thereof; or a pharmaceutically acceptable salt, solvate or prodrug thereof.

One embodiment of this aspect is a compound of formula (I), as set forth above in the Summary of the Invention, wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³, or —R¹¹—N(R⁹)—C(═NR¹²)N(R¹²)R¹³;
R⁵ and R⁶ are the same and are selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R¹⁵, —R¹¹—C(O)OR¹⁴, —R¹¹—OC(O)R⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl, or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl.

Another embodiment of this aspect is a compound of formula (I), as set forth above in the Summary of the Invention, wherein:
n and m are each independently 0, 1 or 2;
R¹ is —S—;
R² is a direct bond;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —S—;
R² is a direct bond;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —S—;
R² is a direct bond;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, selected from the group consisting of: dibenzo[b,d]thiophene-4,6-diylbis(methylene) dicarbamimidothioate; (2-fluorodibenzo[b,d]thiophene-4,6-diyl)bis(methylene) dicarbamimidothioate; (3,7-dibromodibenzo[b,d]thiophene-4,6-diyl)bis(methylene) dicarbamimidothioate; (2-chloro-8-fluorodibenzo[b,d]thiophene-4,6-diyl)bis(methylene) dicarbamimidothioate; and (3-bromodibenzo[b,d]thiophene-4,6-diyl)bis(methylene) dicarbamimidothioate.

Another embodiment of this aspect is a compound of formula (I), as set forth above in the Summary of the Invention, wherein:
n and m are each independently 0, 1 or 2;
R¹ is a direct bond;
R² is —S—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, which is dibenzo[b,d]thiophene-1,9-diylbis(methylene) dicarbamimidothioate.

Another embodiment of this aspect is a compound of formula (I), as set forth above in the Summary of the Invention, wherein:
n and m are each independently 0, 1 or 2;
R¹ is —O—;
R² is a direct bond or —C(O)—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —O—;
R² is a direct bond or —C(O)—;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —O—;
R² is a direct bond or —C(O)—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, selected from the group consisting of: (9-oxo-9H-xanthene-4,5-diyl)bis(methylene) dicarbamimidothioate; dibenzo[b,d]furan-4,6-diylbis(methylene)dicarbamimidothioate; (3,7-dimethyldibenzo[b,d]furan-4,6-diyl)bis(methylene) dicarbamimidothioate; (3,7-dichloroldibenzo[b,d]furan-4,6-diyl)bis(methylene) dicarbamimidothioate; (3,7-dibromodibenzo[b,d]furan-4,6-diyl)bis(methylene) dicarbamimidothioate; (2-fluorodibenzo[b,d]furan-4,6-diyl)bis(methylene) dicarbamimidothioate; and (2,8-dibromodibenzo[b,d]furan-4,6-diyl)bis(methylene) dicarbamimidothioate.

Another embodiment of this aspect is a compound of formula (I), as set forth above in the Summary of the Invention, wherein:
n and m are each independently 0, 1 or 2;
R¹ is a direct bond;
R² is a direct bond;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is a direct bond;
R² is a direct bond;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is a direct bond;
R² is a direct bond;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, selected from the group consisting of: biphenylene-1,8-diylbis(methylene) dicarbamimidothioate; and (3,6-difluorobiphenylene-1,8-diyl)bis(methylene) dicarbamimidothioate.

Another embodiment of this aspect is a compound of formula (I), as set forth above in the Summary of the Invention, wherein:
n and m are each independently 0, 1 or 2;
R¹ is a direct bond;
R² is a direct bond;
R³ and R⁴ are both —R¹¹—S—C(═NR¹²)N(R¹²)R¹³;
R⁵ and R⁶ are both —R¹¹—S—C(═NR¹²)N(R¹²)R¹³;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, which is biphenylene-1,4,5,8-tetrayltetrakis(methylene) tetracarbamimidothioate.

Another embodiment of this aspect is a compound of formula (I), as set forth above in the Summary of the Invention, wherein:
n and m are each independently 0, 1 or 2;
R¹ is —C(O)—;
R² is a direct bond;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —C(O)—;
R² is a direct bond;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —C(O)—;

R² is a direct bond;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, which is 2-(8-carbamimidoylsulfanylmethyl-9-oxo-9H-fluoren-1-ylmethyl)-isothiourea.

Another embodiment of this aspect is a compound of formula (I), as set forth above in the Summary of the Invention, wherein:
n and m are each independently 0, 1 or 2;
R¹ is a direct bond;
R² is —C(O)—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, which is (9-oxo-9H-fluorene-4,5-diyl)bis(methylene)dicarbamimidothioate.

Another embodiment of this aspect is a compound of formula (I), as set forth above in the Summary of the Invention, wherein:
n and m are each independently 0, 1 or 2;
R¹ is —O—;
R² is —C(R⁹)₂—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —O—;
R² is —C(R⁹)₂—;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —O—;
R² is —C(R⁹)₂—;
R³ and R⁴ are both —R¹¹—S—C(═NR²)N(R¹²)R¹³;
R⁵ and R⁶ are both hydrogen;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, which is 2-(2,7-di-tert-butyl-5-carbamimidoylsulfanylmethyl-9,9-dimethyl-9H-xanthen-4-ylmethyl)-isothiourea.

Another embodiment of this aspect is a compound of formula (I), as set forth above in the Summary of the Invention, wherein:
n and m are each independently 0, 1 or 2;
R¹ is —O—;
R² is —S—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —O—;
R² is —S—;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is —O—;
R² is —S—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, which is phenoxathiine-4,6-diylbis(methylene) dicarbamimidothioate.

Another embodiment of this aspect is a compound of formula (I), as set forth above in the Summary of the Invention, wherein:
n and m are each independently 0, 1 or 2;
R¹ is a direct bond;
R² is —CH₂—S—CH₂—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is a direct bond;
R² is —CH₂—S—CH₂—;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, wherein:
n and m are each independently 0, 1 or 2;
R¹ is a direct bond;
R² is —CH₂—S—CH₂—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl; and
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

Another embodiment of this aspect is a compound of formula (I), as set forth above in the Summary of the Invention, which is (5,7-dihydrodibenzo[c,e]thiepine-1,11-diyl)bis(methylene) dicarbamimidothioate.

Another embodiment of this aspect is a compound of formula (I), as set forth above in the Summary of the Invention, wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are different and are each independently selected from —R¹¹—S—C(═NR¹²)N(R¹²)R¹³, —R¹¹—O—C(═NR¹²)N(R¹²)R¹³, R¹¹—C(═NR¹²)N(R¹²)R¹³ or —R¹¹—N(R⁹)—C(═NR¹²)N(R¹²)R¹³;
R⁵ and R⁶ are different and are each independently selected from hydrogen, alkyl, halo, haloalkyl, —R¹¹—CN, —R₁—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R⁵, —R¹¹—C(O)OR¹⁴, —R¹¹—OC(O)R⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl.

Another embodiment of this aspect is a compound of formula (I), as set forth above in the Summary of the Invention, wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are different and are each independently selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, alkyl, halo, haloalkyl, —R¹¹—CN, —R¹¹—NO₂, —R¹¹—N(R¹⁴)₂, —R¹¹—C(O)OR¹⁴, —R¹—C(O)N(R¹⁴)₂—R¹¹—S—C(═NR¹²)N(R¹²)R³, —R¹¹—O—C(═NR²)N(R²)R³, —R¹¹—C(═NR²)N(R²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R⁵, —R¹¹—N(R¹⁴)₂, —R¹″-S(O)ₚR¹⁴, —R¹¹—C(O)R¹⁴, —R¹¹—C(S)R¹⁵, —R¹¹—C(O)OR¹⁴, —R¹¹—OC(O)R¹⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl.

Another embodiment of this aspect is a compound of formula (I), as set forth above in the Summary of the Invention, wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are different and are each independently selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹—S—C(═NR¹²)N(R¹²)R³, —R¹¹—O—C(═NR¹²)N(R²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R¹⁵, —R¹¹—C(O)OR⁴, —R¹¹—OC(O)R⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl.

Another aspect of the invention is a compound of formula (II), as set forth above in the Summary of the Invention, as a stereoisomer, enantiomer, tautomer thereof or mixtures thereof; or a pharmaceutically acceptable salt, solvate or prodrug thereof.

One embodiment of this aspect is a compound of formula (II), as set forth above in the Summary of the Invention, wherein:
q and r are each independently 0, 1 or 2;
R¹⁶ and R¹⁷ are each independently ═C(R²⁴)— or ═N—;
R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are the same and are selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—_—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR², or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)₂, —R²⁵—S(O)R²⁸, R²⁵—C(O)R²⁸, —R²⁵—C(S)R²⁹, —R²⁵—C(O)OR²⁸, —R²⁵—OC(O)R²⁸, —R²⁵—C(S)OR²⁸, —R²⁵—C(O)N(R²⁸)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl.

Another embodiment of this aspect is a compound of formula (II), as set forth above in the Summary of the Invention, wherein:
q and r are each independently 0, 1 or 2;
R¹⁶ and R¹⁷ are each ═C(R²⁴)—;
R¹⁸ and R¹⁹ are the same and are selected from —R²⁵—S—C(═NR²⁶)N(R²⁶)R²⁷, —R⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
R²⁰ and R²¹ are the same and are selected from hydrogen, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
each R²² and R²³ is independently selected from the group consisting of —R²⁵—OR²⁴, alkyl, halo and haloalkyl;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain; and
each R²⁶ and R²⁷ is independently hydrogen, alkyl or —OR²⁴.

Another embodiment of this aspect is a compound of formula (II), as set forth above in the Summary of the Invention, wherein:
q and r are each independently 0, 1 or 2;
R¹⁶ and R¹⁷ are each ═C(R²⁴)—;
R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
R²⁰ and R²¹ are the same and are selected from hydrogen, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
each R²² and R²³ is independently selected from the group consisting of —R²⁵—OR²⁴, alkyl, halo and haloalkyl;
each R²⁴ is hydrogen, alkyl, haloalkyl, alkoxyalkyl, optionally substituted aryl or optionally substituted aralkyl;
each R²⁵ is independently a direct bond or a straight or branched alkylene chain; and
each R²⁶ and R²⁷ is independently hydrogen, alkyl or —OR²⁴.

Another embodiment of this aspect is a compound of formula (II), as set forth above in the Summary of the Invention, wherein:
q and r are each independently 0, 1 or 2;
R¹⁶ and R¹⁷ are each ═C(R²⁴)—;
R¹⁸ and R¹⁹ are both —R²⁵—S—C(═NR²⁶)N(R²⁶)R²⁷;
R²⁰ and R²¹ are both hydrogen;
each R²² and R²³ is independently selected from the group consisting of —R²⁵—OR²⁴, alkyl, halo and haloalkyl;
each R²⁴ is hydrogen, alkyl, haloalkyl, alkoxyalkyl, optionally substituted aryl or optionally substituted aralkyl;
each R²⁵ is independently a direct bond or a straight or branched alkylene chain; and
each R²⁶ and R²⁷ is independently hydrogen, alkyl or —OR²⁴.

Another embodiment of this aspect is a compound of formula (II), as set forth above in the Summary of the Invention, which is anthracene-1,8-diylbis(methylene) dicarbamimidothioate.

Another embodiment of this aspect is a compound of formula (II), as set forth above in the Summary of the Invention, wherein:
q and r are each independently 0, 1 or 2;
R¹⁶ is ═N—;
R¹⁷ is ═C(R²⁴)—;
R¹⁸ and R¹⁹ are the same and are selected from —R²⁵—S—C(═NR²⁶)N(R²⁶)R²⁷, —R²⁵—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
R²⁰ and R²¹ are the same and are selected from hydrogen, —R²⁵—S—C(═NR²⁶)N(R²⁶)R²⁷, —R²⁵, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
each R²² and R²³ is independently selected from the group consisting of —R²⁵—OR²⁴, alkyl, halo and haloalkyl;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain; and
each R²⁶ and R²⁷ is independently hydrogen, alkyl or —OR²⁴.

Another embodiment of this aspect is a compound of formula (II), as set forth above in the Summary of the Invention, wherein:
q and r are each independently 0, 1 or 2;
R¹⁶ is ═N—;
R¹⁷ is ═C(R²⁴)—;
R¹⁸ and R¹⁹ are the same and are selected from —R²⁵—S—C(═NR²⁶)N(R²⁶)R²⁷, —R²⁵—OC(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
R²⁰ and R²¹ are the same and are selected from hydrogen, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
each R²² and R²³ is independently selected from the group consisting of —R²⁵—OR²⁴, alkyl, halo and haloalkyl;
each R²⁴ is hydrogen, alkyl, haloalkyl, alkoxyalkyl, optionally substituted aryl or optionally substituted aralkyl;
each R²⁵ is independently a direct bond or a straight or branched alkylene chain; and
each R²⁶ and R²⁷ is independently hydrogen, alkyl or —OR²⁴.

Another embodiment of this aspect is a compound of formula (II), as set forth above in the Summary of the Invention, wherein:
q and r are each independently 0, 1 or 2;
R¹⁶ is ═N—;
R¹⁷ is ═C(R²⁴)—;
R¹⁸ and R¹⁹ are both —R²⁵—S—C(═NR²⁶)N(R²⁶)R²⁷;
R²⁰ and R²¹ are both hydrogen;
each R²² and R²³ is independently selected from the group consisting of —R²⁵—OR²⁴, alkyl, halo and haloalkyl;
each R²⁴ is hydrogen, alkyl, haloalkyl, alkoxyalkyl, optionally substituted aryl or optionally substituted aralkyl;
each R²⁵ is independently a direct bond or a straight or branched alkylene chain; and
each R²⁶ and R²⁷ is independently hydrogen, alkyl or —OR²⁴.

Another embodiment of this aspect is a compound of formula (I), as set forth above in the Summary of the Invention, selected from the group consisting of: acridine-4,5-diylbis(methylene) dicarbamimidothioate; and (9-methylacridine-4,5-diyl)bis(methylene) dicarbamimidothioate.

Another embodiment of this aspect is a compound of formula (II), as set forth above in the Summary of the Invention, wherein:
q and r are each independently 0, 1 or 2;
R¹⁶ and R¹⁷ are each independently ═C(R²⁴)— or ═N—;
R¹⁸ and R¹⁹ are different and are each independently selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are different and are each independently selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)₂, —R²⁵—S(O)R²⁸, —R²⁵—C(O)R²⁸, —R²⁵—C(S)R²⁹, —R²⁵—C(O)OR²⁸, —R²⁵—OC(O)R²⁸, —R²⁵—C(S)OR²⁸, —R²⁵—C(O)N(R²⁸)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl.

Another embodiment of this aspect is a compound of formula (II), as set forth above in the Summary of the Invention, wherein:
q and r are each independently 0, 1 or 2;
R¹⁶ and R¹⁷ are each independently ═C(R²⁴)— or ═N—;
R¹⁸ and R¹⁹ are different and are each independently selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are the same and are selected from hydrogen, alkyl, halo, haloalkyl, —R²⁵CN, —R²⁵—NO₂—R²⁵—N(R²⁸)₂—R²⁵—C(O)R²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)₂, —R²⁵—S(O)R²⁸, —R²⁵—C(O)R²⁸, —R²⁵—C(S)R²⁹, —R²⁵—C(O)OR²⁸, —R²⁵—OC(O)R²⁸, —R²⁵—C(S)OR²⁸, —R²⁵—C(O)N(R²)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl.

Another embodiment of this aspect is a compound of formula (II), as set forth above in the Summary of the Invention, wherein:
q and r are each independently 0, 1 or 2;
R¹⁶ and R¹⁷ are each independently ═C(R²⁴)— or ═N—;
R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are different and are each independently selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)₂, —R²⁵—S(O)R²⁸, —R²⁵— (O)R²⁸, —R²⁵—C(S)R²⁹, —R²⁵—C(O)OR²⁸, —R²⁵—OC(O)R²⁸, —R²⁵—C(S)OR²⁸, —R²⁵—C(O)N(R²⁸)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)N(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl.

Another aspect of this invention are pharmaceutical compositions comprising a pharmaceutically acceptable excipient and a therapeutically effective amount of a compound of the invention, as set forth above in the Summary of the Invention, as a stereoisomer, enantiomer, tautomer thereof or mixtures thereof, or a pharmaceutically acceptable salt, solvate or prodrug thereof.

One embodiment of this aspect of the invention are pharmaceutical compositions comprising a pharmaceutically acceptable excipient and a therapeutically effective amount of an embodiment of a compound of formula (I), as set forth above, as a stereoisomer, enantiomer, tautomer thereof or mixtures thereof, or a pharmaceutically acceptable salt, solvate or prodrug thereof.

Another embodiment of this aspect of the invention are pharmaceutical compositions comprising a pharmaceutically acceptable excipient and a therapeutically effective amount of an embodiment of a compound of formula (II), as set forth above, as a stereoisomer, enantiomer, tautomer thereof or mixtures thereof, or a pharmaceutically acceptable salt, solvate or prodrug thereof.

Another aspect of the invention are methods for treating an iron disorder in a mammal, preferably a human, or a disease or condition associated with an iron disorder in a mammal, preferably a human, wherein the method comprises administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising an embodiment of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

One embodiment of this aspect is where the disease or condition associated with the iron disorder is due to an accumulation of iron in the body tissues of the mammal.

Another embodiment of this aspect is where the iron disorder is a primary iron overload disorder.

Of this embodiment, a preferred embodiment is where the primary iron overload disorder is independently selected from the group consisting of hereditary hemochromatosis, juvenile hemochromatosis, ferroportin disease, neonatal hemochromatosis, Bantu siderosis, African iron overload, gracile syndrome, ataxia, and Friedreich Ataxia. A more preferred embodiment is where the primary iron overload is hereditary hemochromatosis.

Another embodiment of this aspect is where the iron disorder is a secondary iron overload disorder.

Another embodiment of this aspect is where the iron disorder is transfusional iron overload disorder.

Another embodiment of this aspect is where the disease or condition is independently selected from the group consisting of thalassemia (beta and alpha, major, minor and intermedia), hypochromic microcytic anemia, sickle cell anemia, microcytic iron loading anemia, hereditary sideroblastic anemia, congenital dyserythropoeitic anemia, porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia.

Another embodiment of this aspect is where the disease or condition associated with an iron overload is independently selected from the group consisting of neurodegenerative disease (including ALS, prion diseases, Parkinson's, and Alzheimers), cardiovascular disease (including atherosclerosis, ischemic cerebrovascular disease and ischemic stroke), inflammation (including arthritis and disease progression in viral hepatitis), cancer, insulin resistance, non-alcoholic liver disease, alcoholic liver disease, and infectious disease (including HIV, malaria and Yersinia infections).

Another embodiment of the invention are methods for treating an iron disorder associated with DMT1 activity in a mammal, preferably a human, or for treating a disease or condition associated with DMT1 activity in a mammal, preferably a human, wherein the method comprises administering to the mammal in need thereof a therapeutically effective amount of a compound of the invention, as set forth above, as a stereoisomer, enantiomer, tautomer thereof or mixtures thereof, or a pharmaceutically acceptable salt, solvate or prodrug thereof, or a therapeutically effective amount of a pharmaceutical composition comprising an embodiment of a compound of the invention, as set forth above, as a stereoisomer, enantiomer, tautomer thereof or mixtures thereof, or a pharmaceutically acceptable salt, solvate or prodrug thereof, and a pharmaceutically acceptable excipient.

Of this embodiment, one embodiment is where the DMT1 activity is upregulated (i.e., increased levels of DMT1 activity as compared to normal levels of DMT1 activity).

Of this embodiment, another embodiment is where the therapeutically effective amount administered to the mammal is a DMT1-inhibitory amount.

Specific embodiments of the compounds of the invention are described in more detail below in the following sections.

### Utility and Testing of the Compounds of the Invention

The present invention is directed to compounds and pharmaceutical compositions comprising the compounds, as described herein and above in the Summary of the Invention, which are useful in the treatment of iron disorders in a mammal, preferably a human, by modulating, preferably inhibiting, DMT1 activity.

The term “iron disorder” refers to a condition in a mammal, preferably a human, wherein the level of iron in the body is outside the normal range for the particular mammal (i.e. abnormal iron level), such as an elevated or a decreased iron serum level compared to the normal iron serum level for the mammal or an increased or decreased level of iron in the liver of the mammal as compared to the normal level of iron in the liver in the mammal. Abnormal iron serum levels can be determined by direct measurement of serum iron using a calorimetric assay, or by the standard transferrin saturation assay (which reveals how much iron is bound to the protein that carries iron in the blood), or by the standard serum ferritin assay. For example, transferrin saturation levels of 45% or higher are usually indicative of abnormally high levels of iron in the serum. Abnormal iron levels in the liver can be determined measuring the iron content of the liver from tissue obtained by a liver biopsy or by imaging technique such as MRI and/or SQUID. The degree of iron levels in other tissues (e.g., brain, heart) may also be estimated using these and other imaging techniques. Preferably, for purposes of this invention, an abnormal iron level is an elevated iron level in serum or tissue.

The term “iron disorders” therefore includes both iron deficiency disorders and iron overload disorders. Preferably, the iron disorder is an iron overload disorder, such as primary iron overload disorder (including, but not limited to, hereditary hemochromatosis, juvenile hemochromatosis, ferroportin disease, neonatal hemochromatosis, Bantu siderosis, African iron overload, gracile syndrome, ataxia, and Friedreich Ataxia, as well as all of the anemias listed below in which patients may not be transfused but may become iron overloaded due to increased erythroid drive and the resulting increased iron absorption in the gut) and secondary (or transfusional) iron overload disorder which can be caused by repeated transfusions used to treat a number of distinct anemias, including, but not limited to, thalassemia (beta and alpha, major, minor and intermedia), hypochromic microcytic anemias, sickle cell anemia, microcytic iron loading anemias, hereditary sideroblastic anemias, congenital dyserythropoeitic anemias, porphyria cutanea tarda, pyruvate kinase deficiency, hereditary atransferrinemia, ceruloplasmin deficiency, myelodysplastic syndromes, pulmonary hemosiderosis, aceruloplasminemia and x-linked sideroblastic anemia.

Iron disorders of particular interest in the practice of the invention are iron overload disorders where the level of iron in a mammal is higher than the normal level of iron in the mammal. Such iron overload disorders including, but are not limited to, primary iron overload disorders (including, but not limited to, hereditary hemochromatosis, juvenile hemochromatosis, ferroportin disease, neonatal hemochromatosis, Bantu siderosis, African iron overload, gracile syndrome, ataxia, and Friedreich Ataxia, as well as all of the anemias listed below, in which patients may not be transfused but may become iron overloaded due to increased erythroid drive and the resulting increased iron absorption in the gut), and secondary (transfusional) iron overload disorders (including, but not limited to, thalassemia (beta and alpha, major, minor and intermedia)), hypochromic microcytic anemias, sickle cell anemia, microcytic iron loading anemias, hereditary sideroblastic anemias, congenital dyserythropoeitic anemias, porphyria cutanea tarda, pyruvate kinase deficiency, hereditary atransferrinemia, ceruloplasmin deficiency, myelodysplastic syndromes, pulmonary hemosiderosis, aceruloplasminemia, and x-linked sideroblastic anemia. Iron overload may also be responsible for a portion of the pathology observed in neurodegenerative diseases (including ALS, prion diseases, Parkinson's, Alzheimers), cardiovascular diseases (including atherosclerosis, ischemic cerebrovascular disease and ischemic stroke), inflammatory diseases and conditions (including arthritis and disease progression in viral hepatitis), cancer, insulin resistance, non-alcoholic liver disease, alcoholic liver disease, and infectious disease (including HIV, malaria and Yersinia infections).

The compounds of the invention, and pharmaceutical compositions comprising the compounds of the invention, are useful in treating iron disorders by modulating, preferably inhibiting, DMT1 activity. There is evidence that the upregulation (i.e., increased activity) of DMT1 has a role in iron disorders caused by genetic abnormalities, such as hereditary hemochromatosis. Hereditary hemochromatosis is an iron overload disorder due to intestinal iron hyperabsorption. Hereditary hemochromatosis is characterized by a slow accumulation of iron from the diet to toxic levels resulting in tissue injury and multi-organ malfunction. Patients, typically men, develop symptoms of hemochromatosis in their fourth and fifth decade with variable combinations of cirrhosis, hepatoma, arthritis, hypogonadism, diabetes mellitus and cardiomyopathy. The biochemical profile shows elevated transferrin saturation above 45% and a high serum ferritin. The underlying genetic defect in hereditary hemochromatosis is a mutation in the hemochromatosis gene (HFE) on chromosome 6p21. 90% of Northern Europeans with hereditary hemochromatosis are homozygous for a single missense mutation, C282Y in exon 4 of the HFE gene.

DMT1 activity has also been implicated in the etiology and pathophysiology of hypochromic microcytic anemias, thalassemia, microcytic iron loading anemias, hereditary sideroblastic anemias, hereditary hypochromic anemias, congenital dyserythropoietic anemias, pyruvate kinase deficiency, hereditary atransferrinemia, and certain myelodysplastic syndromes, as there is a direct correlation between the degree of iron limited anemia, increased DMT1 expression in the duodenum and, by extension, increased iron absorption via DMT1 (Morgan et al., Blood Cells Molecules and Diseases, 2002, 29:384-399).

There is also evidence that DMT1 has a role in iron disorders such as acquired iron overload. The risk factors for acquired iron overload might include for example excessive ingestion of red meat, iron supplements or foods that are iron fortified. Acquired iron overload can also occur from the use of iron cookware, drinking unpurified tap water, use of oral contraceptives, blood transfusions and cigarette smoking. DMT1 pattern of expression and function supports it as a candidate target for the treatment of acquired iron overload and other related maladies.

In addition to the small intestine, DMT1 is also highly expressed in the kidney suggesting a role in renal iron handling and possibly reabsorption of filtered iron (Ferguson et al., Am. J. Physiol. Renal. Physiol., 2001, 280: F803-F814) and is also involved in the delivery of iron to peripheral tissues by transferrin (Fleming et al., Proc. Natl. Acad. Sci., 1998, 85:1148-1153). DMT1 inhibitors, when dosed in a fashion that increases their systemic exposure, may be useful in an acute unloading of iron via the urine, by inhibiting DMT1 expressed in the kidney.

DMT1 may also play a role in regulating iron flux to the brain. As there is some indication that iron overload in the brain may play a role in brain pathology, such as Alzheimer's, DMT1 inhibitors may act to reduce the amount of iron absorbed by the brain, when dosed in a fashion that increases their systemic exposure and allows them to play a role at the blood brain barrier or within the brain (Lehmann et al., 2006, J. Med. Genet., 2006, 43(10):e52; Schenck et al., Top. Magn Reson. Imaging., 2006, 17(1):41-50).

Studies show that mutant mice that are defective in DMT1 activity (mk/mk) develop hyprochromic microcytic anemia, a severe form of iron deficiency anemia, due to a defect in intestinal iron absorption. In contrast, the hfe^{-/-} knockout mouse model of hereditary hemochromatosis is characterized by an enhanced intestinal iron uptake and total body iron overload. The hfe^{-/-}:mk/mk double mutant mouse, which carries mutations in both the HFE and DMT1 genes, fails to load iron, indicating that hemochromatosis (hfe^{-/-}) can be prevented by blocking the flux of iron through the DMT1 protein (Levy et al., J. Clin. Invest., 2000, 105:1209-16). In addition, studies of human patients with hereditary hemochromatosis show that DMT1 is inappropriately upregulated at the intestinal brush border. This aberrant excessive expression of DMT1 in hereditary hemochromatosis is fundamental to the primary pathophysiology of this condition (Zoller et al., Gastroenterology, 2001, 120:1412-1419). These findings have made DMT1 a therapeutic target for the treatment of iron overload disorders in general, and, in particular, for the treatment of hereditary hemochromatosis. In further support of DMT1 as a therapeutic target in the treatment of iron overload, it has been shown in clinical studies that the majority of the excess iron burden is absorbed in the form of ferrous (non-heme) iron, as opposed to heme-iron (Lynch et al., Blood, 1989, 74:2187-2193).

While not wishing to be bound to any particular mechanism of action, the compounds of the invention, and pharmaceutical compositions comprising the compounds of the invention, are useful in treating iron disorders by directly interacting with a region of the DMT1 protein that modulates or controls iron flux. A direct interaction is supported by the fact that the compounds are not potent inhibitors of cation flux in the closely related transporter Natural Resistance-Associated Macrophage Protein-1 (NRAMP1). In general, the compounds of the invention modulate the activity of DMT1 downwards, thereby inhibiting the ability of DMT1 to uptake non-heme iron across the cellular membrane. The compounds of the invention are therefore considered to be DMT1 inhibitors and are therefore useful in treating iron disorders which are ameliorated by the modulation, preferably the inhibition, of DMT1 activity. The compounds of the invention, as DMT1 inhibitors, are also useful in reducing normal or slightly abnormal iron serum levels in a mammal, preferably a human, wherein the reduction of iron serum levels provides a therapeutic benefit to the mammal, preferably a human, such as neuroprotective activity after a stroke.

The compounds of the invention, and pharmaceutical compositions comprising the compounds of the invention, are also useful in treating or preventing symptoms, diseases and/or conditions in a mammal associated with hereditary hemochromatosis due to accumulation of iron in body tissues such as arthritis, liver disease, heart disease, impotence, early menopause, abnormal skin pigmentation, thyroid deficiency, damage to pancreas, diabetes, and damage to adrenal gland (Sheth et al., Annu. Rev. Med., 2000, 51:443-464).

The compounds of the invention, and pharmaceutical compositions comprising the compounds of the invention, are also useful in treating or preventing other forms of hemochromatosis including, but are not limited to, juvenile hemochromatosis and neonatal hemochromatosis. Juvenile hemochromatosis has a much earlier onset and exhibits more severe symptoms such as endocrine dysfunction, joint disease, and cardiac abnormalities due to excessive iron deposition from an early age. Neonatal hemochromatosis is a rare fetal gestational condition that results in iron accumulation in the liver of the fetus.

The compounds of the invention, and pharmaceutical compositions comprising the compounds of the invention, are also useful in treating or preventing transfusional iron overload. Chronic blood transfusion is the established therapy for thalassaemia major, bone marrow failure and complications of sickle cell anaemia and other related disorders. With hypertransfusion, the systemic iron load accumulates. Because there is no natural way for the body to eliminate the iron, the excess iron in the transfused blood builds up to cause iron overload and becomes toxic to tissues and organs, particularly the liver, heart, and pancreas. Transfusional iron overload typically results in the patient's premature death from organ failure. The transfusional iron overload is unfortunately augmented by increased iron absorption, which is the natural attempt of the body to increase iron levels in order to promote erythropoiesis, which is itself compromised by the disease states above. Decreased absorption of iron by the inhibition of DMT1 activity may reduce the iron overload related to the transfusional iron overload and supports the use of DMT1 inhibitors for the treatment of this disease.

In addition, due to iron's ability to generate reactive oxygen species (free radicals), which can result in inflammation and tissue damage, the compounds of the invention, and pharmaceutical compositions comprising the compounds of the invention, may also be useful as anti-inflammatory or neuroprotective agents due to their ability to reduce iron serum levels by the modulation, preferably inhibition, of DMT1 activity.

The general value of the compounds of the invention, and pharmaceutical compositions comprising the compounds of the invention, in modulating, preferably inhibiting, DMT1 activity can be determined using the assays described herein or below in the Biological Assays section. Alternatively, the general value of the compounds of the invention, and pharmaceutical compositions comprising the compounds of the invention, in treating iron disorders in humans may be established in industry standard animal models for demonstrating the efficacy of compounds in treating iron disorders.

In particular, identification of the compounds of the invention ability to modulate, preferably to inhibit, DMT1 activity, can be assessed using a variety of in vitro and in vivo assays, for measuring uptake of reduced iron (Fe²⁺). One such protocol involves the screening of chemical agents for ability to modulate the activity of DMT1 thereby identifying it as a modulating agent. The in vitro activity of DMT1 can be measured in cell based assays by either directly measuring iron flux (using a radioactively labelled iron ⁵⁵Fe) or by measuring the fluorescence of a cell permeable iron fluorophore such as calcein. Stable cell lines overexpressing DMT1 are exposed to ⁵⁵Fe or loaded with calcein and then compound is applied. Decreased flux of ⁵⁵Fe or lack of fluorescence quenching indicates that the given modulator has inhibited DMT1 function (Picard et al., J. Biol. Chem., 2000, 275(46):35738-45 and Wetli et al., Chem. Biol. 2006 September; 13(9):965-72). Alternatively, in another format electrophysiological techniques can be used to measure the current or iron or other metals traversing the cell membrane with DMT1 in a Xenopus oocyte or other cell based system (Gunshin et al., Nature, 1997, 31; 388(6641):482-8).

Other assays may involve intestinal cells or tissues which express endogenous DMT1, using the same detection techniques such as fluorescence, radiolabelled iron or electrophysiology. A human Caco2 cell line can be used for such assays (Alvarez-Hernandez et al., Biochimica. et. Biophysica. Acta., 1991, 1070:205-208). These assays can be performed in the presence of desferroxamine to render the cells iron deficient and upregulate DMT1 expression. Alternatively, intestinal tissue may be used, either as gut rings which will take up iron (Raja et al., Cell. Biochemistry and Function, 1987, 5:69-76; Leppert et al., J. of Pharm. Sci., 1994, 83:976-981), or as gut slices ex vivo (Vaghefi et al., Reprod. Nutr. Dev., 1998, 38:559-566) where iron flux across the epithelial layer can be assessed in an Ussing chamber. In these assays, tissue can be excised from iron replete or iron deficient animals. In addition, the heme versus non-heme iron absorptive capacity of the tissue can be measured.

These assays can be carried out in transfected cells, or cell or tissue endogenously expressing the channel of interest in a natural endogenous setting or in a recombinant setting. Other methods of testing the compounds disclosed herein are also readily known and available to those skilled in the art.

Compounds of the invention can also be tested in a variety of in vivo models so as to determine if they alleviate a particular iron disorder in a mammal, particularly an iron overload disorder, with minimal adverse events. The assays described herein and below in the Biological Assays Section are useful in assessing the in vivo activity of the compounds of the invention.

For example, a typical rat model of iron overload disorder can be created by establishing an iron deficient state in the rate, which will then cause the upregulation of DMT1 expression and activity, resulting in increased iron absorption. These models can be used to demonstrate that compounds of the invention have the ability to modulate, preferably inhibit, the activity of DMT1 as demonstrated by the increase in serum iron levels in the iron-deficient rat. Iron deficiency is induced in these rat models in order to mimic the DMT1 over-expression and iron hyperabsorption observed in humans having iron overload disorders such as hereditary hemochromatosis as well as humans suffering from thalassemia.

Alternatively, an iron deficient, and therefore hyperabsorptive state, may be induced by dietary means, such as, for example, treatment with phenylhydrazine, or by phlebotomy (Refino et al., Am. J. Clin. Nutr. 1983, 37:904-909; Redondo et al., Lab. Animal Sci. 1995, 45:578-583; Frazer et al., Gastroenterology, 2002, 123:835-844). Alternatively, iron absorption can also be stimulated by creating an hypoxic state to stimulate erythropoiesis (Raja et al., Br. J. Haematol., 1988, 68:373-378). In these models, a compound's efficacy can be assessed by measuring reduced iron flux via the duodenum acutely or by monitoring whether chronic exposure to a compound causes a decrease in the amount of iron loading as measured by serum iron, transferrin saturation, ferritin and liver iron. Alternatively, iron flux in these animals can be measured by tracing the absorption of radioactive iron administered orally. These experiments can also be performed in iron replete animals, although changes in these parameters will be less pronounced and therefore compound efficacy will be more difficult to judge.

Genetic rat models of iron overload offers another format to show efficacy of DMT1 inhibitors in preventing further iron loading. These models are applicable to a variety of iron disorders such as hereditary hemochromatosis (Levy et al., Blood, 1999, 94:9-11), juvenile hemochromatosis (Huang et al., J. Clin. Invest., 2005 115:2187-2191), beta-2-microglobulin (de Sousa et al., Immun. Lett., 1994, 39:105-111), thalassemia (Ciavatta et al., Proc. Nat. Acad. Sci., 1995, 92: 9259-9263), hypotransferrinmia (Craven et al., Proc. Nat. Acad. Sci., 1987, 84(10):3457-61) and other hypochromic microcytic anemias. A compound's efficacy can be assessed by measuring reduced iron flux via the duodenum acutely or by monitoring whether chronic exposure to a compound causes a decrease in the amount of iron loading as judged by serum iron, transferrin saturation, ferritin and liver iron. Alternatively, iron flux in these animals can be measured by tracing the absorption of radioactive iron administered orally.

Typically, a successful therapeutic agent of the present invention will meet some or all of the following criteria. Oral availability should be at less than 5%. Animal model efficacy is less than about 0.1 μg to about 100 mg/Kg body weight and the target human dose is between 0.1 μg to about 100 mg/Kg body weight, although doses outside of this range may be acceptable (“mg/Kg” means milligrams of compound per kilogram of body mass of the subject to whom it is being administered). The therapeutic index (or ratio of toxic dose to therapeutic dose) should be greater than 100. The potency (as expressed by IC₅₀ value) should be less than 10 μM, preferably below 1 μM and most preferably below 50 nM. The IC₅₀ (“Inhibitory Concentration—50%”) is a measure of the amount of compound required to achieve 50% inhibition of DMT1, over a specific time period, in an assay of the invention.

In another use of the invention, the compounds of the invention can be used in in vitro or in vivo studies as exemplary agents for comparative purposes to find other compounds useful in the treatment of an iron disorder or diseases or conditions associated with an iron disorder.

In another use of the invention, the compounds of the invention can be used in the preparation of a medicament for the treatment of an iron disorder in a mammal or for the treatment of a disease or condition associated with an iron disorder in a mammal.

### Pharmaceutical Compositions of the Invention and Administration

The present invention also relates to pharmaceutical composition containing the compounds of the invention disclosed herein. In one embodiment, the present invention relates to a composition comprising compounds of the invention in a pharmaceutically acceptable carrier, excipient or diluent and in an amount effective to modulate, preferably inhibit, DMT1 in order to treat iron disorders when administered to an animal, preferably a mammal, most preferably a human patient.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., N.J. current edition).

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 10% by weight of the compound prior to dilution of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 Kg mammal) from about 0.001 mg/Kg (i.e., 0.07 mg) to about 100 mg/Kg (i.e., 7.0 g); preferably a therapeutically effective dose is (for a 70 Kg mammal) from about 0.01 mg/Kg (i.e., 0.7 mg) to about 50 mg/Kg (i.e., 3.5 g); more preferably a therapeutically effective dose is (for a 70 Kg mammal) from about 1 mg/Kg (i.e., 70 mg) to about 25 mg/Kg (i.e., 1.75 g).

The ranges of effective doses provided herein are not intended to be limiting and represent preferred dose ranges. However, the most preferred dosage will be tailored to the individual subject, as is understood and determinable by one skilled in the relevant arts. (see, e.g., Berkow et al., eds., The Merck Manual, 16^{th} edition, Merck and Co., Rahway, N.J., 1992; Goodman et al., eds., Goodman and Gilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

The total dose required for each treatment can be administered by multiple doses or in a single dose over the course of the day, if desired. Generally, treatment is initiated with smaller dosages, which are less than the optimum dose of the compound. Thereafter, the dosage is increased by small increments until the optimum effect under the circumstances is reached. The diagnostic pharmaceutical compound or composition can be administered alone or in conjunction with other diagnostics and/or pharmaceuticals directed to the pathology, or directed to other symptoms of the pathology. The recipients of administration of compounds and/or compositions of the invention can be any vertebrate animal, such as mammals. Among mammals, the preferred recipients are mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the preferred recipients are turkeys, chickens and other members of the same order. The most preferred recipients are humans.

For topical applications, it is preferred to administer an effective amount of a pharmaceutical composition according to the invention to target area, e.g., skin surfaces, mucous membranes, and the like, which are adjacent to peripheral neurons which are to be treated. This amount will generally range from about 0.0001 mg to about 1 g of a compound of the invention per application, depending upon the area to be treated, whether the use is diagnostic, prophylactic or therapeutic, the severity of the symptoms, and the nature of the topical vehicle employed. A preferred topical preparation is an ointment, wherein about 0.001 to about 50 mg of active ingredient is used per cc of ointment base. The pharmaceutical composition can be formulated as transdermal compositions or transdermal delivery devices (“patches”). Such compositions include, for example, a backing, active compound reservoir, a control membrane, liner and contact adhesive. Such transdermal patches may be used to provide continuous pulsatile, or on demand delivery of the compounds of the present invention as desired.

The compositions of the invention can be formulated so as to provide quick, sustained or delayed release of the active ingredient after administration to the patient by employing procedures known in the art. Controlled release drug delivery systems include osmotic pump systems and dissolutional systems containing polymer-coated reservoirs or drug-polymer matrix formulations. Examples of controlled release systems are given in U.S. Pat. Nos. 3,845,770 and 4,326,525 and in P. J. Kuzma et al, Regional Anesthesia 22 (6): 543-551 (1997), all of which are incorporated herein by reference.

The compositions of the invention can also be delivered through intra-nasal drug delivery systems for local, systemic, and nose-to-brain medical therapies. Controlled Particle Dispersion (CPD)™ technology, traditional nasal spray bottles, inhalers or nebulizers are known by those skilled in the art to provide effective local and systemic delivery of drugs by targeting the olfactory region and paranasal sinuses.

The invention also relates to an intravaginal shell or core drug delivery device suitable for administration to the human or animal female. The device may be comprised of the active pharmaceutical ingredient in a polymer matrix, surrounded by a sheath, and capable of releasing the compound in a substantially zero order pattern on a daily basis similar to devises used to apply testosterone as described in PCT Patent No. WO 98/50016.

Current methods for ocular delivery include topical administration (eye drops), subconjunctival injections, periocular injections, intravitreal injections, surgical implants and iontophoresis (uses a small electrical current to transportionized drugs into and through body tissues). Those skilled in the art would combine the best suited excipients with the compound for safe and effective intra-occular administration.

The most suitable route will depend on the nature and severity of the condition being treated. Those skilled in the art are also familiar with determining administration methods (oral, intravenous, inhalation, sub-cutaneous, rectal etc.), dosage forms, suitable pharmaceutical excipients and other matters relevant to the delivery of the compounds to a subject in need thereof.

### Combination Therapy

The compounds of the invention may be usefully combined with one or more other compounds of the invention or one or more other therapeutic agent or as any combination thereof, in the treatment of iron disorders. For example, a compound of the invention may be administered simultaneously, sequentially or separately in combination with other therapeutic agents, including, but not limited to iron chelators, e.g. deferasirox (ICL-670), deferiprone, and desferroxamine; erythropoietin (EPO), e.g. rh-EPO. In addition, compounds of the invention, as inhibitors of DMT1 activity, could also be combined with phlebotomy therapy for the treatment of iron overload disorders.

As used herein “combination” refers to any mixture or permutation of one or more compounds of the invention and one or more other compounds of the invention or one or more additional therapeutic agent. Unless the context makes clear otherwise, “combination” may include simultaneous or sequentially delivery of a compound of the invention with one or more therapeutic agents. Unless the context makes clear otherwise, “combination” may include dosage forms of a compound of the invention with another therapeutic agent. Unless the context makes clear otherwise, “combination” may include routes of administration of a compound of the invention with another therapeutic agent. Unless the context makes clear otherwise, “combination” may include formulations of a compound of the invention with another therapeutic agent. Dosage forms, routes of administration and pharmaceutical compositions include, but are not limited to, those described herein.

### Kits-of-Parts

The present invention also provides kits that contain a pharmaceutical composition which includes one or more compounds of the invention. The kit also includes instructions for the use of the pharmaceutical composition for treating iron disorders as well as other utilities as disclosed herein. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

### Preparation of the Compounds of the Invention

The following Reaction Schemes illustrate methods to make compounds of formula (I):

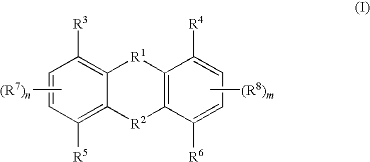

wherein n, m, R¹, R², R³, R⁴, R⁵, R⁶, R⁷ and R⁸ are as defined above in the Summary of the Invention for compounds of formula (I), as a stereoisomer, enantiomer, tautomer thereof or mixtures thereof; or a pharmaceutically acceptable salt, solvate or prodrug thereof.

The following Reaction Schemes also illustrate methods to make compounds of formula (II):

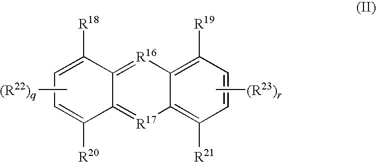

wherein q, r, R¹⁵, R¹⁶, R¹⁷, R¹⁸, R¹⁹, R²⁰, R²¹ and R²² are as defined above in the Summary of the Invention for compounds of formula (II), as a stereoisomer, enantiomer, tautomer thereof or mixtures thereof; or a pharmaceutically acceptable salt, solvate or prodrug thereof.

It is understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters.

Protecting groups may be added or removed in accordance with standard techniques, which are known to one skilled in the art and as described herein.

The use of protecting groups is described in detail in Greene, T. W. and P. G. M. Wuts, Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

The following Reaction Schemes illustrate methods to make compounds of this invention. It is understood that one skilled in the art would be able to make these compounds by similar methods or by methods known to one skilled in the art. It is also understood that one skilled in the art would be able to make in a similar manner as described below other compounds of the invention not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Smith, M. B. and J. March, Advanced Organic Chemistry: Reactions, Mechanisms, and Structure, 5th edition (Wiley, December 2000)) or prepared as described herein.

### A. Preparation of Compounds of Formula (Ia)

Compounds of formula (Ia) are compounds of formula (I), as set forth above in the Summary of the Invention, where R¹ is —C(O)—, R² is a direct bond, R³ and R⁴ are both —R¹¹—S—C(═NR¹²)N(R¹²)R¹³ (where R¹¹ is methylene, each R¹² is hydrogen and R¹³ is hydrogen), and n, m, R⁵, R⁶, R⁷ and R⁸ are each as described above in the Summary of the Invention, and are prepared as set forth below in Reaction Scheme 1:

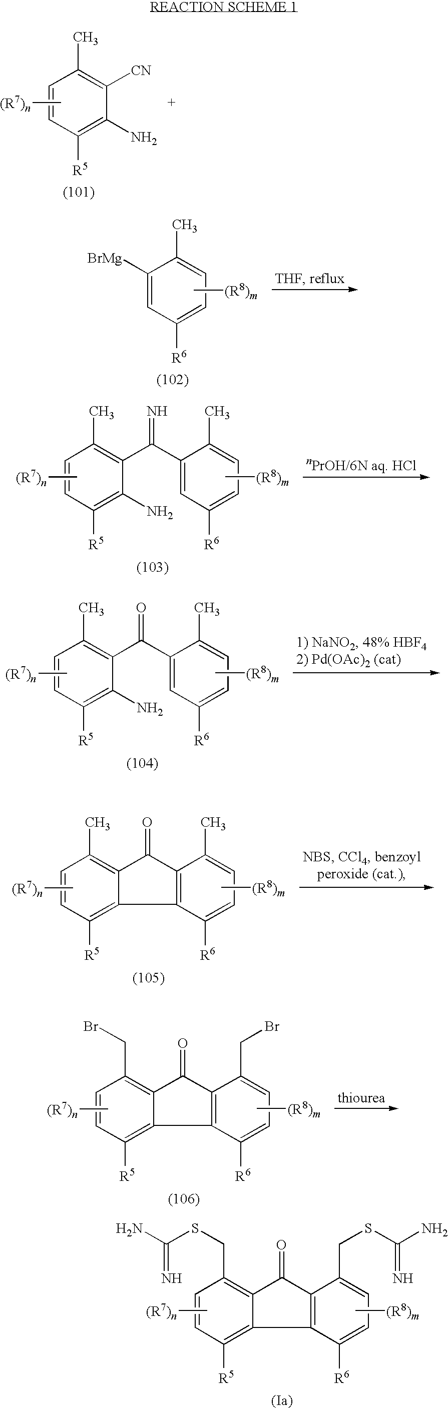

Compounds of formula (101) and formula (102) are commercially available, or can be prepared by methods known to one skilled in the art or by the methods disclosed herein.

In general, the compounds of formula (Ia) can be synthesized by the method shown above in Reaction Scheme 1 by first reacting a cyano compound of formula (101) with a Gringard reagent of formula (102) under reflux to afford the imine compound of formula (103), which is converted to the ketone compound of formula (104) under acidic conditions. Compound of formula (104) is treated with a diazotization reagent, such as, but not limited to, sodium nitrite, at low temperature in the presence of tetrafluoroboric acid. Intramolecular cyclization of the diazonium salt in the presence of catalytic amount of palladium(II) acetate affords the fluorenone compound of formula (105). Bromination of compound of formula (105) with N-bromosuccinimide generates the di-bromo compound of formula (106) and subsequent displacement of the bromo groups with thiourea affords the compound of formula (Ia) of the invention.

### B. Preparation of Compounds of Formula (Ib)

Compounds of formula (Ib) are compounds of formula (I), as set forth above in the Summary of the Invention, where R¹ is —O— or —S—, R² is a direct bond, R³ and R⁴ are both —R¹¹—S—C(═NR¹²)N(R¹²)R¹³ (where R¹¹ is methylene, each R¹² is hydrogen and R¹³ is hydrogen), and n, m, R⁵, R⁶, R⁷ and R⁸ are each as described above in the Summary of the Invention, and X is chloro or bromo, and are prepared as set forth below in Reaction Scheme 2 where n, m, R¹, R⁵, R⁶, R⁷ and R⁸ are as described above:

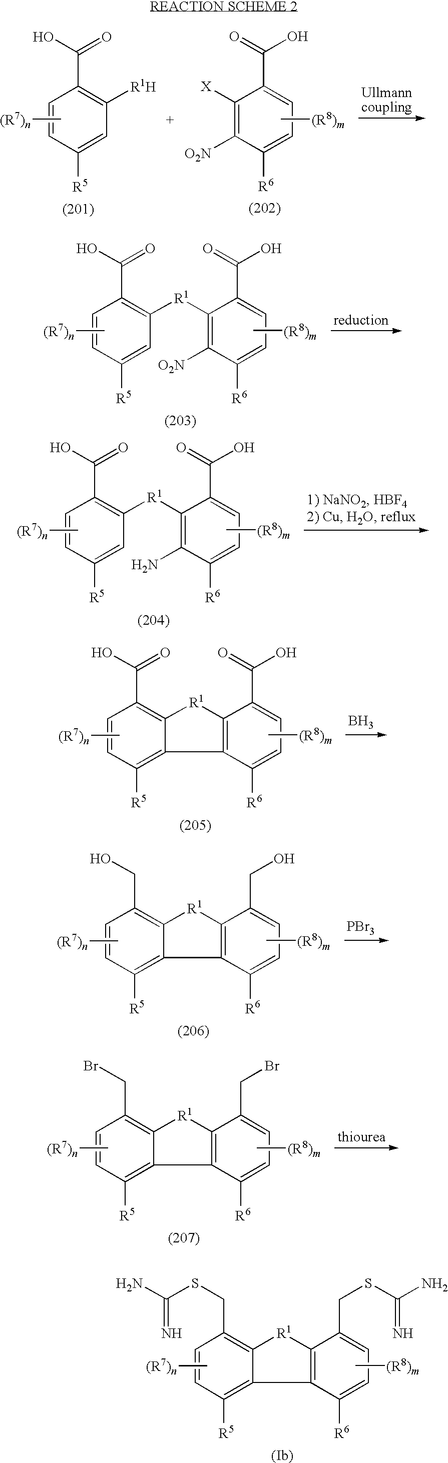

Compounds of formula (201) and formula (202) are commercially available, or can be prepared by methods known to one skilled in the art or by the methods disclosed herein.

In general, the compounds of formula (Ib) can be synthesized by the method shown above in Reaction Scheme 2 by first coupling the compound of formula (201) coupled with a compound of formula (202) under Ullmann coupling conditions in the presence of copper at 120-200° C. to afford the di-aryl compound of formula (203). Reduction of the nitro group of the compound of formula (203) with a reducing agent, such as, but not limited to, zinc, affords the amino compound of formula (204), which is treated with a diazotization reagent, such as, but not limited to, sodium nitrite, at low temperature, such as 0° C., in the presence of tetrafluoroboric acid to lead to the intramolecular cyclization of the diazonium salt in the presence of copper at reflux to afford the compound of formula (205). Reduction of the di-acid compound of formula (205) with a reducing agent, such as, but not limited to, borane-tetrahydrofuran complex, generates the di-alcohol compound of formula (206). Bromination of the compound of formula (206) with a brominating agent, such as, but not limited to, PBr₃, affords the di-bromo compound of formula (207). Subsequent displacement of the bromo groups in the compound of formula (207) with thiourea affords the compound of formula (Ib) of the invention.

Alternatively, compounds of formula (Ib), as described above, can be prepared as set forth below in Reaction Scheme 3 where n, m, R¹, R⁵, R⁶, R⁷ and R⁸ are as described above for compounds of formula (Ib):

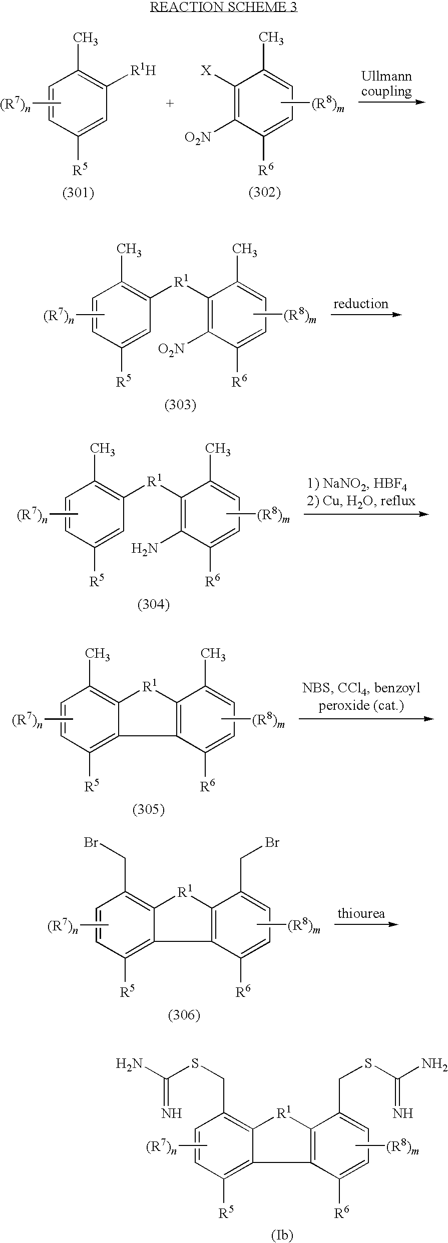

Compounds of formula (301) and formula (302) are commercially available, or can be prepared by methods known to one skilled in the art or by the methods disclosed herein.

In general, compounds of formula (Ib) can be synthesized by the method shown above in Reaction Scheme 3 by first coupling a compound of formula (301) with a compound of formula (302) under Ullmann coupling conditions in the presence of copper at 120-200° C. to afford the compound of formula (303). Reduction of the nitro group of the compound of formula (303) with a reducing agent, such as, but not limited to, zinc, affords the amino compound of formula (304), which is treated with a diazotization reagent, such as, but not limited to, sodium nitrite, at low temperature, such as 0° C., in the presence of tetrafluoroboric acid to lead to the intramolecular cyclization of the diazonium salt in the presence of copper at reflux to afford the compound of formula (305). Bromination of the compound of formula (305) with N-bromosuccinimide affords the di-bromo compound of formula (306). Subsequent displacement of the bromo groups of the compound of formula (306) with thiourea affords the compound of formula (Ib), as described above.

### C. Preparation of Compounds of Formula (Ic)

Compounds of formula (Ic) are compounds of formula (I), as set forth above in the Summary of the Invention, where R¹ is —O— or —S—, R² is a direct bond, R³ and R⁴ are both —R¹¹—S—C(═NR¹²)N(R¹²)R¹³ (where R¹¹ is methylene, each R¹² is hydrogen and R¹³ is hydrogen), R⁵ and R⁶ are both hydrogen, and n is 1 and R⁷ is methyl, and m is 1 and R⁸ is methyl, and are prepared as set forth below in Reaction Scheme 4 where R¹ is as described above:

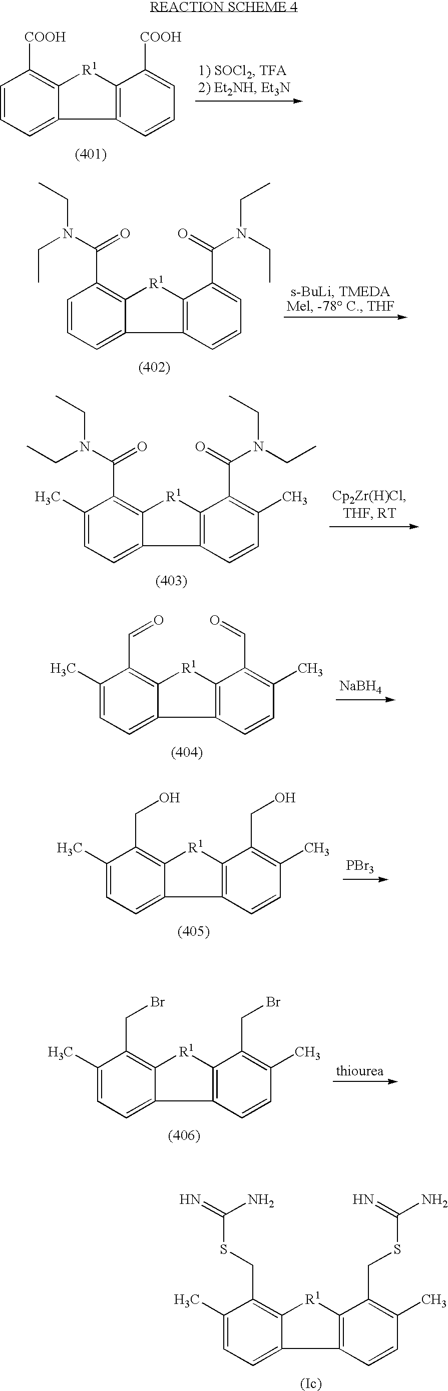

Compounds of formula (401) can be prepared by methods known to one skilled in the art or by the methods disclosed herein.

In general, compounds of formula (Ic) can be synthesized by the method shown above in Reaction Scheme 4 by first treating the di-acid compound of formula (401) with diethylamine under standard amide formation conditions known to the one skilled in the art to afford the amide compound of formula (402). The compound of formula (402) is methylated at the ortho positions relative to the amide groups under directed ortho-metalation (DoM) conditions known to one skilled in the art to generate compound of formula (403). Reduction of the amide groups of the compound of formula (403) using Schwarts reagent affords the aldehyde intermediate of formula (404), which is further reduced to the corresponding alcohol compound of formula (405) by a reducing agent, such as, but not limited to, sodium borohydride, to afford a compound of formula (405). Bromination of the compound of formula (405) with phosphorus tribromide affords the di-bromo compound of formula (406). Subsequent displacement of the bromo groups on the compound of formula (406) with thiourea affords the compound of formula (Ic) of the invention.

### D. Preparation of Compounds of Formula (Id)

Compounds of formula (Id) are compounds of formula (I), as set forth above in the Summary of the Invention, where R¹ and R² are each a direct bond, R³ and R⁴ are both —R¹¹—S—C(═NR¹²)N(R¹²)R¹³ (where R¹¹ is methylene, each R¹² is hydrogen and R¹³ is hydrogen), R⁵ and R⁶ are both hydrogen, and n, m, R⁷ and R⁸ are as described above in the Summary of the Invention, and are prepared as set forth below in Reaction Scheme 5 where n, m, R⁷ and R⁸ are as described above:

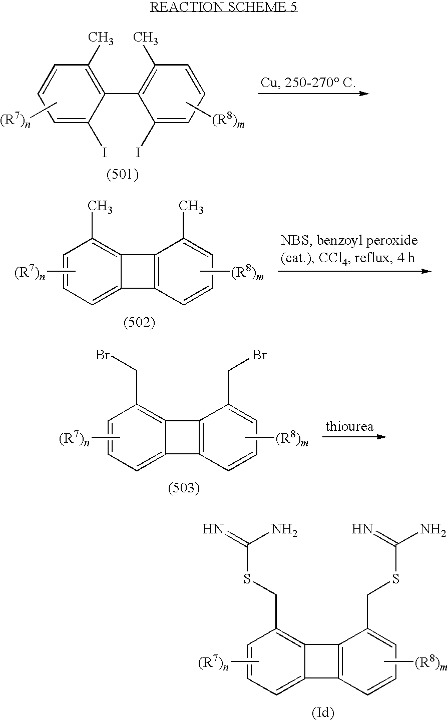

Compounds of formula (501) are commercially available, or can be prepared by methods known to one skilled in the art or by the methods disclosed herein.

In general, compounds of formula (Id) can be synthesized by the method shown above in Reaction Scheme 5 by first cyclizing the di-iodo compound of formula (501) intramolecularly in the presence of copper at 250-270° C. to afford the biphenylene compound of formula (502). Bromination of the compound (502) with N-bromosuccinimide affords the di-bromo compound of formula (503). Subsequent displacement of the bromo groups of the compound of formula (503) with thiourea affords the compound of formula (Id) of the invention.

### E. Preparation of Compounds of Formula (Ie)

Compounds of formula (Ie) are compounds of formula (I), as set forth above in the Summary of the Invention, where R¹ is —O— or —S—, R² is a direct bond, R³ and R⁴ are both —R¹¹—S—C(═NR¹²)N(R¹²)R¹³ (where R¹¹ is methylene, each R¹² is hydrogen and R¹³ is hydrogen), R⁵ and R⁶ are both hydrogen, n and m are each 1, and R⁷ and R⁸ are as described above in the Summary of the Invention, and can be prepared as described below in Reaction Scheme 6 where R¹ is as described above, and R⁷ and R⁸ are alkyl, R¹⁴ is alkyl and Y is I or Br:

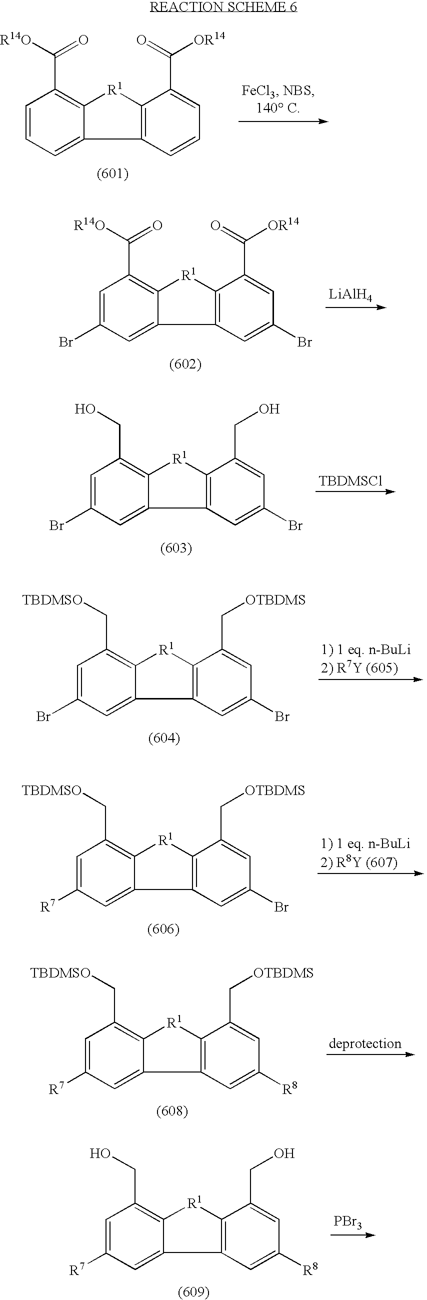

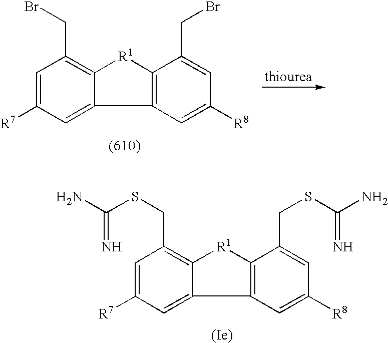

Compounds of formula (601) can be prepared by methods known to one skilled in the art or by the methods disclosed herein.

In general, compounds of formula (Ie) can be synthesized by the method shown above in Reaction Scheme 6 by first brominating the compound of formula (601) with N-bromosuccinimide in the presence of FeCl₃ at 140° C. to afford the di-bromo compound of formula (602). Reduction of the ester groups of compound of formula (602) by lithium aluminum hydride generates the di-alcohol compound of formula (603). Protection of the alcohol groups of the compound of formula (603) with tert-butyldimethylsilyl (TBDMS) groups generates the compound of formula (604). The compound of formula (604) undergoes metal-halogen exchange reaction with 1 equivalent of n-butyl lithium and followed by quenching with an electrophile of formula (605) to generate the compound of formula (606), which undergoes another metal-halogen exchange reaction with 1 equivalent of n-butyl lithium followed by quenching with an electrophile of formula (607) to generate the compound of formula (608). Removal of the TBDMS protecting groups under standard conditions known to one skilled in the art generates the compound of formula (609). Bromination of the compound of formula (609) with phosphorus tribromide affords the di-bromo compound of formula (610). Subsequent displacement of the bromo groups on the compound of formula (610) with thiourea affords the compound of formula (Ie) of the invention.

Alternatively, the compounds of formula (Ie), as described above, can be synthesized following the general procedure described below in Reaction Scheme 7 where R¹ is —O— or —S—, R⁷ and R⁸ are each optionally substituted aryl and X is Cl or Br:

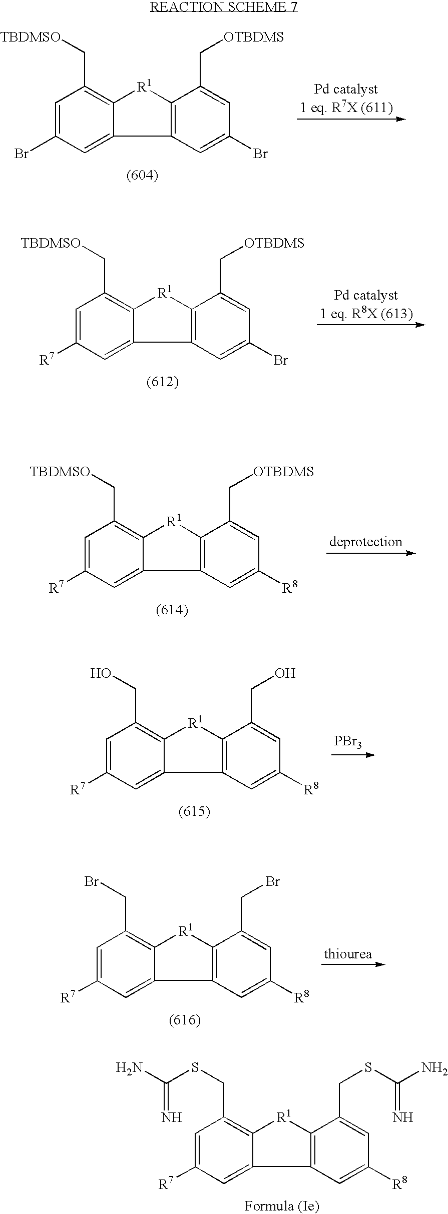

Compounds of formula (604) can be prepared according to methods known to one skilled in the art or by methods disclosed herein.

In general, compounds of formula (Ie) can be synthesized by the method shown above in Reaction Scheme 7 by first treating the di-bromo compound of formula (604) with 1 equivalent of a compound of formula (611) under standard metal-catalyzed cross-coupling reaction conditions known to one skilled in the art, such as palladium catalyzed cross-coupling reaction conditions, to generate the compound of formula (612). The compound of formula (612) is treated with 1 equivalent of compound (613) under standard metal catalyzed cross coupling reaction conditions to generate compound (614). Removal of the TBDMS protecting groups on the compound of formula (614) under standard conditions known to one skilled in the art generates the compound of formula (615). Bromination of the compound of formula (615) with phosphorus tribromide affords the di-bromo compound of formula (616). Subsequent displacement of the bromo groups on the compound of formula (616) with thiourea affords the compound of formula (Ie) of the invention.

### F. Preparation of Compounds of Formula (If)

Compounds of formula (If) are compounds of formula (I), as set forth above in the Summary of the Invention, where n and m are both 0, R¹ is a direct bond, R² is —O— or —S—, R³ and R⁴ are both —R¹¹—S—C(═NR¹²)N(R¹²)R¹³ (where R¹¹ is methylene, each R¹² is hydrogen and R¹³ is hydrogen), and R⁵ and R⁶ are both hydrogen, and can be prepared as described below in Reaction Scheme 8 where R² is as described above:

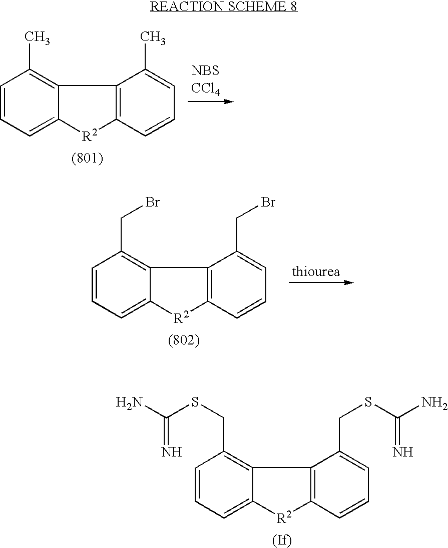

Compound of formula (801) is commercially available or can be prepared according to methods known to one skilled in the art or by methods disclosed herein.

In general, compounds of formula (If) can be synthesized by the method shown above in Reaction Scheme 8 by first brominating a compound of formula (801) with N-bromosuccinimide to afford the di-bromo compound of formula (802). Subsequent displacement of the bromo groups on the compound of formula (802) with thiourea affords the compound of formula (If) of the invention.

### G. Preparation of Compounds of Formula (IIa)

Compounds of formula (IIa) are compounds of formula (II), as set forth above in the Summary of the Invention, where R¹⁶ is ═N—, R¹⁷ is ═C(R²⁴)— (where R²⁴ is as described above in the Summary of the Invention), R²⁰ and R²¹ are both hydrogen, q, r, R²², R²³ and R²⁴ are as described above in the Summary of the Invention, and R¹⁸ and R¹⁹ are both —R²⁵—S—C(═NR²⁶)N(R²⁶)R²⁷ (where R²⁵ is methylene, each R²⁶ is hydrogen and R²⁷ is hydrogen), and can be prepared as described below in Reaction Scheme 9 where q, r, R²², R²³ and R²⁴ are as described above:

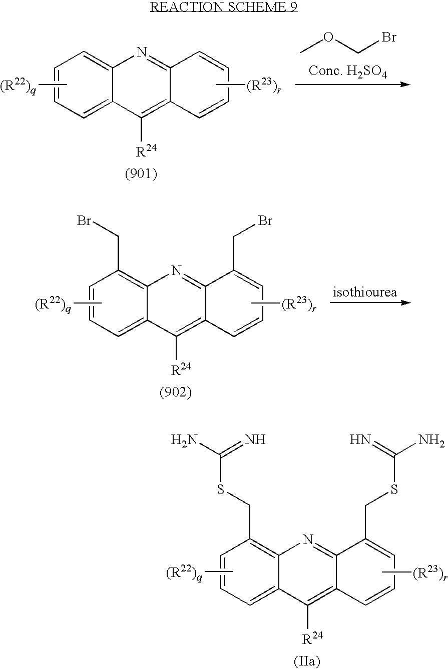

Compound of formula (901) can be prepared according to methods known to one skilled in the art utilizing commercially available starting materials or by methods disclosed herein.

In general, compounds of formula (IIa) can be synthesized by the method shown above in Reaction Scheme 9 by first treating the acridine compound of formula (901) with bromo(methoxy)methane in concentrated sulfuric acid to afford the di-bromo compound of formula (902). Displacement of the bromo groups on the compound of formula (902) with thiourea affords the compound of formula (IIa) of the invention.

All compounds of the invention as prepared above and below which exist in free base or acid form may be converted to their pharmaceutically acceptable salt by treatment with the appropriate inorganic or organic base or acid by methods known to one skilled in the art. Salts of the compounds prepared herein may be converted to their free base or acid by standard techniques known to one skilled in the art.

The following Preparations, which are directed to the preparation of intermediates used in the preparation of the compounds of formula (I) and formula (II), and the following Examples, which are directed to the preparation of the compounds of formula (I) and formula (II), are provided as a guide to assist in the practice of the invention, and are not intended as a limitation on the scope of the invention.

### Preparation 1

### Preparation of 3,7-dibromo-4,6-dimethyldibenzo[b,d]thiophene

To a mixture of 4,6-dimethyldibenzo[b,d]thiophene (0.21 g, 1.00 mmol) in acetic acid (3 mL) was added bromine (0.11 mL, 2.20 mmol) at ambient temperature. The reaction mixture was stirred at ambient temperature for 16 h. The solid was collected by filtration and recrystallized from ethyl acetate to afford 3,7-dibromo-4,6-dimethyldibenzo[b,d]thiophene as a colorless solid in 73% yield (0.27 g): ¹H NMR (300 MHz, CDCl₃) δ 7.77 (d, J=8.4 Hz, 2H), 7.62 (d, J=8.4 Hz, 2H), 2.67 (s, 6H).

### Preparation 1.1

### Preparation of 3,7-dibromo-4,6-dimethyldibenzo[b,d]furan

Following the procedure as described in Preparation 1, making non-critical variations using 4,6-dimethyldibenzo[b,d]furan to replace 4,6-dimethyldibenzo[b,d]-thiophene, 3,7-dibromo-4,6-dimethyldibenzo[b,d]furan was obtained as a colorless solid in 43% yield: ¹H NMR (300 MHz, CDCl₃) δ 7.56 (d, J=8.3 Hz, 2H), 7.48 (d, J=8.3 Hz, 2H), 2.62 (s, 6H).

### Preparation 2

### Preparation of 3-bromo-4,6-dimethyldibenzo[b,d]thiophene

To a mixture of 4,6-dimethyldibenzo[b,d]thiophene (0.83 g, 4.0 mmol) in acetic acid (3 mL) was added bromine (0.21 mL, 4.0 mmol) at ambient temperature. The reaction mixture was stirred at ambient temperature for 16 h. The solid obtained was collected by filtration and recrystallized from ethyl acetate to afford 3-bromo-4,6-dimethyldibenzo[b,d]thiophene as a colorless solid in 34% yield (0.40 g): ¹H NMR (300 MHz, CDCl₃) δ 7.95 (d, J=7.8 Hz, 1H), 7.82 (d, J=8.4 Hz, 1H), 7.62 (d, J=8.4 Hz, 1H), 7.40 (t, J=7.5 Hz, 1H), 7.29 (d, J=6.9 Hz, 1H), 2.69 (s, 3H), 2.61 (s, 3H).

### Preparation 3

### Preparation of 4,6-dimethyldibenzo[b,d]furan

A solution of dibenzo[b,d]furan (5.00 g, 29.70 mmol) in diethyl ether (200 mL) was flushed with argon for one hour before the addition of N,N,N′,N′-tetramethylethylenediamine (11.1 mL, 74.3 mmol), followed by the addition of s-butyllithium (53.1 mL of 1.4 M solution, 74.3 mmol) slowly at −78° C. The mixture was stirred at ambient temperature for 16 h and methyl iodide (9.3 mL, 148.6 mmol) was added. The resulting mixture was stirred at ambient temperature for another 16 h, followed by the addition of saturated ammonium chloride solution (100 mL) to quench the reaction. The mixture was extracted with diethyl ether (3×100 mL). The combined organic layers was dried over anhydrous sodium sulfate and filtered. The filtrate was concentrated in vacuo. The residue was recrystallized from methanol to afford 4,6-dimethyldibenzo[b,d]furan as a colorless solid in 43% yield (2.50 g): ¹H NMR (300 MHz, CDCl₃) δ 7.78-7.72 (m, 2H), 7.26-7.17 (m, 4H), 2.61 (s, 6H).

### Preparation 4

### Preparation of 3,7-difluoro-4,6-dimethyldibenzo[b,d]furan

To a solution of 3,7-dibromo-4,6-dimethyldibenzo[b,d]furan (1.34 g, 3.81 mmol) in tetrahydrofuran (20 mL) at −78° C. was added n-butyl lithium in cyclohexane (5.0 mL of 1.6 M solution, 8.0 mmol). The reaction mixture was stirred at −78° C. for 1 h, followed by the addition of N-fluorobenzenesulfonimide (3.60 g, 11.40 mmol) in tetrahydrofuran (10 mL). The reaction mixture was stirred at −78° C. for 4 h, followed by the addition of saturated ammonium chloride solution to quench the reaction. The mixture was diluted with ethyl acetate (100 mL), and washed with water and brine. The organic layer was dried over anhydrous sodium sulfate, filtered and the filtrate was concentrated to dryness. The residue was purified by column chromatography (hexane) to afford 3,7-difluoro-4,6-dimethyldibenzo[b,d]furan as a colorless solid in 54% yield (0.48 g): ¹H NMR (300 MHz, CDCl₃) δ 7.62 (dd, J=8.4, 5.4 Hz, 2H), 7.04 (dd, J=9.9, 8.4 Hz, 2H), 2.51 (s, 6H).

### Preparation 4.1

### Preparation of 3,7-dichloro-4,6-dimethyldibenzo[b,d]furan

Following the procedure as described in Preparation 4, making non-critical variation using hexachloroethane to replace N-fluorobenzenesulfonimide, 3,7-dichloro-4,6-dimethyldibenzo[b,d]furan was obtained as a colorless solid in 38% yield: ¹H NMR (300 MHz, CDCl₃) δ 7.63 (d, J=8.4 Hz, 2H), 7.33 (d, J=8.4 Hz, 2H), 2.62 (s, 6H).

### Preparation 5

### Preparation of 2-fluoro-4,6-dimethyldibenzo[b,d]furan

### A. Preparation of 2-(4-fluoro-2-methylphenoxy)-1-methyl-3-nitrobenzene

To a solution of 4-fluoro-2-methylphenol (3.78 g, 30.00 mmol) and 2-bromo-3-nitrotoluene (4.32 g, 20.00 mmol) in dioxane (40 mL) were added Cu(I) iodide (0.76 g, 4.00 mmol), N,N-dimethylglycine hydrochloride (1.67 g, 12.00 mmol) and cesium carbonate (13.00 g, 40.00 mmol). The reaction mixture was heated to 120° C. in a sealed steel bomb for 16 h and cooled to ambient temperature. The cooled mixture was diluted with ethyl acetate (600 mL), washed with aqueous saturated sodium bicarbonate (2×50 mL) and brine (2×50 mL), dried over anhydrous sodium sulfate and filtered. The filtrate was concentrated in vacuo and the residue was purified by column chromatography eluted with hexane to afford 2-(4-fluoro-2-methylphenoxy)-1-methyl-3-nitrobenzene as a colorless solid (1.75 g, 34%): ¹H NMR (300 MHz, CDCl₃) δ 7.80 (dd, J=8.4, 1.5 Hz, 1H), 7.50 (dd, J=7.5, 0.9 Hz, 1H), 7.27 (dd, J=7.5, 7.5 Hz, 1H), 6.95 (dd, J=8.7, 3.0 Hz, 1H), 6.68 (ddd, J=8.1, 8.1, 3.0 Hz, 1H), 6.23 (dd, J=9.0, 4.5 Hz, 1H), 2.40 (s, 3H), 2.22 (s, 3H).

### B. Preparation of 2-(4-fluoro-2-methylphenoxy)-3-methylaniline

To a stirred solution of 2-(4-fluoro-2-methylphenoxy)-1-methyl-3-nitrobenzene (1.75 g, 6.70 mmol) in acetic acid (12.0 mL) was added a few drops of concentrated hydrochloric acid and zinc dust (3.27 g, 50.00 mmol) at 0° C. The mixture was stirred at ambient temperature for 16 h and filtered. The filtrate was evaporated in vacuo. The residue was taken into ethyl acetate and washed with saturated sodium bicarbonate solution. The organic layer was dried over anhydrous sodium sulfate, filtered, and the filtrate was concentrated to dryness to yield 2-(4-fluoro-2-methylphenoxy)-3-methylaniline as a colorless liquid in 99% yield (1.55 g): ¹H NMR (300 MHz, CDCl₃) δ 7.00-6.91 (m, 2H), 6.74-6.64 (m, 3H), 6.35 (dd, J=9.0, 4.5 Hz, 1H), 4.41-4.00 (br m, 2H), 2.42 (s, 3H), 2.04 (s, 3H); MS (ES+) m/z 232.3 (M+1).

### C. Preparation of 2-fluoro-4,6-dimethyldibenzo[b,d]furan

To an ice cold solution of 2-(4-fluoro-2-methylphenoxy)-3-methylaniline (1.55 g, 6.70 mmol) in tetrahydrofuran (20 mL) were added 48% tetrafluoroboric acid solution (12 mL) and a solution of sodium nitrate (0.55 g, 8.02 mmol) in water (3 mL). The reaction mixture was stirred at 0° C. for 30 min, followed by the addition of palladium acetate (0.01 g). The reaction mixture was heated to 60-70° C. for 2 h, diluted with ethyl acetate (100 mL), and washed with water and brine. The organic layer was dried over anhydrous sodium sulfate, filtered and the filtrate was concentrated to dryness. The residue was purified by column chromatography eluted with hexane to afford 2-fluoro-4,6-dimethyldibenzo[b,d]furan as a colorless solid in 10% yield (0.15 g): ¹H NMR (300 MHz, CDCl₃) δ 7.79-7.70 (m, 1H), 7.46-7.39 (m, 1H), 7.33-7.25 (m, 3H), 2.64 (s, 3H), 2.63 (s, 3H).

### Preparation 6

### Preparation of 4,6-bis(bromomethyl)-2-fluorodibenzo[b,d]thiophene

### A. Preparation of 2-(2-carboxy-4-fluorophenylthio)-3-nitrobenzoic acid

5-Fluorothiosalicilic acid (1.00 g, 5.85 mmol) and 2-bromo-3-nitrobenzoic acid (1.44 g, 5.85 mmol) were dissolved in a solution of potassium carbonate (2.43 g, 17.6 mmol) in water (8.0 mL) and powdered copper (0.38 g, 5.85 mmol) was added. The reaction mixture was heated in a sealed tube at 150° C. for 10 min, cooled to ambient temperature and filtered. The filtrate was acidified with concentrated hydrochloric acid to yield 2-(2-carboxy-4-fluorophenylthio)-3-nitrobenzoic acid in 73% yield (1.45 g): ¹H NMR (300 MHz, DMSO-d₆) δ 13.62 (br s, 2H), 8.09 (dd, J=7.8, 1.4 Hz, 1H), 7.95 (dd, J=7.8, 1.4 Hz, 1H), 7.80 (dd, J=7.8, 7.8 Hz, 1H), 7.63 (dd, J=9.0, 3.0 Hz, 1H), 7.28 (ddd, J=9.0, 7.8, 3.0 Hz, 1H), 6.66 (dd, J=9.0, 5.1 Hz, 1H).

### B. Preparation of (5-fluoro-2-(2-(hydroxymethyl)-6-nitrophenylthio)phenyl)methanol

To a stirred solution of 2-(2-carboxy-6-nitrophenylsulfanyl)-5-fluorobenzoic acid (1.45 g, 4.30 mmol) in tetrahydrofuran (55 mL) was added borane tetrahydrofuran complex solution (14.0 mL of 1 M solution in tetrahydrofuran). The reaction mixture was stirred at ambient temperature overnight. The reaction was quenched by the addition of methanol (15 mL). The solvent was evaporated in vacuo and the residue was taken into ethyl acetate (75 mL). This solution was washed with water and sodium bicarbonate, dried over sodium sulfate, filtered and concentrated in vacuo to yield crude (5-fluoro-2-(2-(hydroxymethyl)-6-nitrophenylthio)phenyl)methanol as an dark oil in 73% yield (1.02 g). MS (ES+) m/z 292.1 (M−17).

### C. Preparation of (3-amino-2-(4-fluoro-2-(hydroxymethyl)phenylthio)phenyl)methanol

To a stirred solution of (5-fluoro-2-(2-(hydroxymethyl)-6-nitrophenylthio)phenyl)methanol (1.00 g, 3.23 mmol) in methanol (25 mL) were added acetic acid (2.0 mL) and a few drops of concentrated hydrochloric acid. Zinc dust (3.50 g, 53.3 mmol) was added. The mixture was stirred at ambient temperature overnight and filtered. The filtrate was concentrated in vacuo and the residue was taken into ethyl acetate and washed with saturated sodium bicarbonate solution. The organic layer was separated, dried over anhydrous sodium sulfate and filtered. The filtrate was concentrated in vacuo to afford (3-amino-2-(4-fluoro-2-(hydroxymethyl)phenylthio)phenyl)methanol in 83% yield (0.75 g) as an oil: ¹H NMR (300 MHz, DMSO-d₆) δ 7.25-7.10 (m, 2H), 7.02-6.76 (m, 1H), 6.78 (d, J=8.0 Hz, 1H), 6.69 (d, J=8.0 Hz, 1H), 6.47 (dd, J=8.6, 5.5 Hz, 1H), 5.46 (t, J=5.5 Hz, 1H), 5.36 (s, 2H), 5.02 (t, J=5.5 Hz, 1H), 4.59 (d, J=5.5 Hz, 2H), 4.42 (d, J=5.5 Hz, 2H).

### D. Preparation of (2-fluorodibenzo[b,d]thiophene-4,6-diyl)dimethanol

(3-Amino-2-(4-fluoro-2-(hydroxymethyl)phenylthio)phenyl)methanol (0.75 g, 2.69 mmol) was dissolved in 25% sulfuric acid (25.0 mL) and sodium nitrite (1.20 g, 17.5 mmol) solution was added at 0° C. Copper powder (1.02 g, 16.1 mmol) was added portionwise to the stirred solution, and the mixture was kept at ambient temperature for 3 h, and then refluxed for 10 min. The product was extracted with ethyl acetate and triturated with hexane to yield (2-fluorodibenzo[b,d]thiophene-4,6-diyl)dimethanol as an off-white solid in 19% yield (0.14 g): ¹H NMR (300 MHz, DMSO-d₆) δ 8.26 (dd, J=6.8, 2.5 Hz, 1H), 8.13 (dd, J=9.5, 2.5 Hz, 1H), 7.40-7.50 (m, 2H), 7.33 (dd, J=9.5, 2.5 Hz, 1H), 5.68 (t, J=5.6 Hz, 1H), 5.54 (t, J=5.6 Hz, 1H), 4.75 (s, 2H), 4.73 (s, 2H).

### E. Preparation of 4,6-bis(bromomethyl)-2-fluorodibenzo[b,d]thiophene

To a stirred suspension of (2-fluorodibenzo[b,d]thiophene-4,6-diyl)dimethanol (0.14 g, 0.51 mmol) in a mixture of dry ether (10 mL) and dichloromethane (10 mL) was added phosphorus tribromide (0.28 g, 1.04 mmol) in one portion. The mixture was stirred at ambient temperature for 16 h, washed with water and dried over sodium sulfate and filtered through a short pad of silica gel. The solvent of the filtrate was removed in vacuo and 4,6-bis(bromomethyl)-2-fluorodibenzo[b,d]thiophene was obtained as a white solid in 45% yield (0.091 g): ¹H NMR (300 MHz, DMSO-d₆) δ 8.82 (dd, J=7.7, 2.5 Hz, 1H), 8.33 (d, J=7.7 Hz, 1H), 7.49-7.32 (m, 3H), 4.81 (s, 2H), 4.78 (s, 2H).

### Preparation 7

### Preparation of 4,6-bis(bromomethyl)-2-chloro-8-fluorodibenzo[b,d]thiophene

### A. Preparation of 2-(2-carboxy-4-fluorophenylthio)-5-chloro-3-nitrobenzoic acid

To a mixture of potassium carbonate (3.52 g, 25.5 mmol) in water (70 mL) and powdered copper (0.11 g, 1.65 mmol) were added 5-fluorothiosalicilic acid ethyl ester (2.00 g, 10.0 mmol) and 3-nitro-2,5-dichlorobenzoic acid (2.36 g, 10.00 mmol). The reaction mixture was heated at 90° C. for 12 h, cooled to ambient temperature, and filtered. The filtrate was acidified with concentrated hydrochloric acid and 2-(2-carboxy-4-fluorophenylthio)-5-chloro-3-nitrobenzoic acid was isolated by filtration as a white solid in 82% yield (3.10 g): ¹H NMR (300 MHz, DMSO-d₆) δ 13.76 (s, 2H), 8.36 (d, J=2.3 Hz, 1H), 8.08 (d, J=2.3 Hz, 1H), 7.64 (dd, J=9.0, 3.0 Hz, 1H), 7.28 (ddd, J=9.0, 8.2, 3.0 Hz, 1H), 6.73 (dd, J=9.0, 5.0 Hz, 1H).

### B. Preparation of (5-chloro-2-(4-fluoro-2-(hydroxymethyl)phenylthio)-3-nitrophenyl)methanol

To a stirred solution of 2-(2-carboxy-4-fluorophenylsulfanyl)-5-chloro-3-nitrobenzoic acid (3.05 g, 8.21 mmol) in tetrahydrofuran (85 mL) was added borane tetrahydrofuran complex solution (24.0 mL of 1 M solution in tetrahydrofuran). The mixture was stirred at ambient temperature overnight. The reaction was quenched by the addition of methanol (15 mL) and acetic acid (6 mL) and then stirred for 16 h. All solvents were removed in vacuo and the residue was taken into ethyl acetate (75 mL). This solution was washed with water and sodium bicarbonate, dried over sodium sulfate, filtered and concentrated in vacuo to afford (5-chloro-2-(4-fluoro-2-(hydroxymethyl)phenylthio)-3-nitrophenyl)methanol as an oil in 99% yield (2.80 g). MS (ES+) m/z 327.1 (M−17).

### C. Preparation of (2-(2-amino-4-chloro-6-(hydroxymethyl)phenylthio)-5-fluorophenyl)methanol

To a stirred solution of (5-chloro-2-(4-fluoro-2-(hydroxymethyl)phenylthio)-3-nitrophenyl)methanol (2.80 g, 8.15 mmol) in methanol (75 mL) was added acetic acid (12 mL) and a few drops of concentrated hydrochloric acid, followed by zinc dust (3.99 g, 61.1 mmol). The mixture was stirred at ambient temperature overnight, filtered and the filtrate was concentrated in vacuo. The residue was taken into ethyl acetate and washed with saturated sodium bicarbonate solution. The organic layer was separated, dried over sodium sulfate and filtered. The filtrate was concentrated in vacuo and the residue was purified by flash chromatography to yield (2-(2-amino-4-chloro-6-(hydroxymethyl)phenylthio)-5-fluorophenyl)methanol in 59% yield (1.50 g): ¹H NMR (300 MHz, DMSO-d₆) δ 7.20 (dd, J=2.9, 9.9 Hz, 1H), 7.02-6.79 (m, 1H), 6.75-6.73 (m, 2H), 6.48 (dd, J=8.6, 5.4 Hz, 1H), 5.71 (s, 2H), 5.48 (br s, 1H), 5.22 (br s, 1H), 4.58 (s, 2H), 4.39 (s, 2H).

### D. Preparation of (2-chloro-8-fluorodibenzo[b,d]thiophene-4,6-diyl)dimethanol

(2-(2-amino-4-chloro-6-(hydroxymethyl)phenylthio)-5-fluorophenyl)methanol (2.00 g, 6.37 mmol) was dissolved in 25% sulfuric acid (50 mL), followed by the addition of sodium nitrite (2.25 g, 31.90 mmol) solution at 0° C. Copper powder (2.03 g, 31.90 mmol) was added portionwise to the stirred solution. The reaction mixture was kept at ambient temperature for 3 h and then boiled for 10 min. The product was extracted with ethyl acetate and triturated with hexane to yield (2-chloro-8-fluorodibenzo[b,d]thiophene-4,6-diyl)dimethanol as an off-white solid in 6.5% yield (0.12 g): ¹H NMR (300 MHz, DMSO-d₆) δ 8.43 (d, J=2.0 Hz, 1H), 8.23 (dd, J=9.5, 2.5 Hz, 1H), 7.51 (d, J=2.0 Hz, 1H), 7.36 (dd, J=9.5, 2.5 Hz, 1H), 5.70 (dd, J=10.5, 5.4 Hz, 2H), 4.73 (s, 4H).

### E. Preparation of 4,6-bis(bromomethyl)-2-chloro-8-fluorodibenzo[b,d]thiophene

To a stirred suspension of (2-chloro-8-fluorodibenzo[b,d]thiophene-4,6-diyl)dimethanol (0.10 g, 0.34 mmol) in dry dichloromethane (10 mL) was added phosphorus tribromide (0.18 g, 0.69 mmol) in one portion. The mixture was stirred at ambient temperature for 16 h, washed with water and dried over sodium sulfate and filtered through a short pad of silica gel. The solvent of the filtrate was removed in vacuo to afford 4,6-bis(bromomethyl)-2-chloro-8-fluorodibenzo[b,d]thiophene as a colorless solid in 80% yield (0.12 g): ¹H NMR (300 MHz, DMSO-d₆) δ 8.57 (d, J=2.0 Hz, 1H), 8.39 (dd, J=9.3, 2.5 Hz, 1H), 7.81 (d, J=2.0 Hz, 1H), 7.67 (dd, J=9.3, 2.5 Hz, 1H), 4.97 (s, 4H).

### Preparation 8

### Preparation of 4,6-bis(bromomethyl)phenoxathiine

### A. Preparation of phenoxathiine-4,6-diyldimethanol

To a stirred solution of phenoxathiine-4,6-dicarboxylic acid (0.15 g, 0.52 mmol) in tetrahydrofuran (25.0 mL) was added borane tetrahydrofuran complex solution (3.0 mL of 1 M solution in tetrahydrofuran). The mixture was stirred at ambient temperature overnight. The reaction was quenched by the addition of methanol (5 mL) and acetic acid (1 mL). The mixture was then taken into ethyl acetate (75 mL) and washed with saturated sodium bicarbonate solution. The organic layer was separated, dried over sodium sulfate and filtered. The filtrate was concentrated in vacuo and the viscous oil was purified by flash chromatography on silica gel to provide phenoxathiine-4,6-diyldimethanol as a white solid in 99% yield (0.14 g): ¹H NMR (300 MHz, DMSO-d₆) δ 7.2-7.3 (m, 2H), 7.12 (dd, J=1.9, 7.7 Hz, 2H), 7.07 (dd, J=6.5, 13.9 Hz, 2H), 5.33 (s, 2H), 4.63 (s, 4H).

### B. Preparation of 4,6-bis(bromomethyl)phenoxathiine

To a stirred suspension of phenoxathiine-4,6-diyldimethanol (0.14 g, 0.52 mmol) in dry ether (30 mL) was added phosphorus tribromide (0.42 g, 1.56 mmol) in one portion. The mixture was stirred for 16 h at ambient temperature, washed with water and filtered through a short pad of silica gel. The solvent of the filtrate was removed in vacuo to afford 4,6-bis(bromomethyl)phenoxathiine as a colorless solid in 85% yield (0.16 g): ¹H NMR (300 MHz, DMSO-d₆) δ 7.34 (dd, J=1.5, 7.6 Hz, 2H), 7.24 (dd, J=1.5, 7.6 Hz, 2H), 7.08 (dd, J=7.6, 7.6 Hz, 2H), 4.84 (s, 4H).

### Preparation 9

### Preparation of 1,8-bis(bromomethyl)-9H-fluoren-9-one

### A. Preparation of 2-(imino(o-tolyl)methyl)-3-methylaniline

To a cooled (0° C.) solution of 2-amino-6-methylbenzonitrile (5.00 g, 37.81 mmol) in anhydrous tetrahydrofuran (50 mL) was added dropwise a solution of 2-methylphenylmagnesium bromide (77.5 mL of a 2 M solution in diethyl ether, 155.0 mmol). The resultant mixture was then heated at reflux for 16 h and allowed to cool to ambient temperature. The reaction mixture was then poured into ice-water (500 mL) and concentrated hydrochloric acid (100 mL) was added. The mixture was transferred to a separatory funnel and was washed with diethyl ether (3×100 mL). The aqueous phase was rendered alkaline by the addition of solid sodium hydroxide (8.0 g) and was extracted with dichloromethane (3×150 mL). The combined dichloromethane extract was washed with brine (150 mL), dried over sodium sulfate and filtered. The filtrate was concentrated in vacuo to dryness to afford 2-(imino(o-tolyl)methyl)-3-methylaniline: MS (ES+) m/z 225.3 (M+1).

### B. Preparation of (2-amino-6-methylphenyl)(o-tolyl)methanone

A mixture of 2-(imino(o-tolyl)methyl)-3-methylaniline in 1-propanol (30 mL) and 6 N aqueous hydrochloric acid (45 mL) was heated at reflux for 16 h and was subsequently cooled to 0° C. The reaction mixture was rendered alkaline by the addition of solid sodium hydroxide (5.0 g) and was transferred to a separatory funnel. The reaction mixture was extracted with dichloromethane (3×150 mL). The combined organic extract was washed with water (3×100 mL) and brine (100 mL), dried over sodium sulfate, filtered and concentrated in vacuo to dryness. The residue was purified by column chromatography eluted with dichloromethane to afford (2-amino-6-methylphenyl)(o-tolyl)methanone as an off-white solid in 29% yield (2.50 g): ¹H NMR (300 MHz, CDCl₃) δ 7.39-7.33 (m, 2H), 7.25 (d, J=7.5 Hz, 1H), 7.17 (dd, J=7.5, 7.5 Hz, 1H), 7.08 (dd, J=7.5, 7.5 Hz, 1H), 6.57 (d, J=7.9 Hz, 1H), 6.50 (d, J=7.9 Hz, 1H), 4.45 (br s, 2H), 2.50 (s, 3H), 1.87 (s, 3H); MS (ES+) m/z 226.3 (M+1).

### C. Preparation of 1,8-dimethyl-9H-fluoren-9-one

To a cooled (0° C.) solution of (2-amino-6-methylphenyl)(o-tolyl)methanone (0.50 g, 2.21 mmol) in tetrahydrofuran (5.0 mL) and 48% aqueous tetrafluoroboric acid (5.0 mL) was added dropwise a solution of sodium nitrite (0.17 g, 2.4 mmol) in water (3.0 mL). The reaction mixture was stirred for 1 h at 0° C., followed by the addition of palladium(II) acetate (0.005 g, 0.02 mmol). The reaction mixture was gradually warmed to 60° C. over 15 min and was held at 60° C. for a further 15 min and cooled to ambient temperature. The precipitate was collected by suction filtration, washed with water (10 mL) and hexanes (10 mL), air-dried and dried under high vacuum. This preparation was repeated three times and the combined material from the four batches was triturated with hexanes (25 mL) to afford 1,8-dimethyl-9H-fluoren-9-one as a yellow solid in 29% yield (0.53 g): ¹H NMR (300 MHz, CDCl₃) δ 7.38-7.26 (m, 4H), 7.04 (d, J=7.8 Hz, 2H), 2.62 (s, 6H).

### D. Preparation of 1,8-bis(bromomethyl)-9H-fluoren-9-one

To a solution of 1,8-dimethyl-9H-fluoren-9-one (0.34 g, 1.52 mmol) in carbon tetrachloride (12.0 mL) was added N-bromosuccinimide (0.54 g, 3.04 mmol) and dibenzoyl peroxide (0.015 g, 0.061 mmol). The reaction mixture was heated at reflux for 6 h, cooled to ambient temperature, diluted with dichloromethane (50 mL) and washed with water (3×50 mL). The organic phase was dried over sodium sulfate, filtered and the filtrate was concentrated in vacuo to dryness. The residue was triturated in boiling chloroform (10 mL) and the solid was collected by suction filtration, washed with ice-cold chloroform (10 mL) and air-dried to afford 1,8-bis(bromomethyl)-9H-fluoren-9-one as a yellow solid in 40% yield (0.22 g): ¹H NMR (300 MHz, DMSO-d₆) δ 7.75-7.67 (m, 2H), 7.63-7.58 (m, 2H), 7.53-7.47 (m, 2H), 5.01 (s, 4H).

### Preparation 10

### Preparation of dimethyl 2,7-di-tert-butyl-9,9-dimethyl-9H-xanthene-4,5-dicarboxylate

To a stirred solution of 2,7-di-tert-butyl-9,9-dimethyl-9H-xanthene-4,5-dicarboxylic acid (1.00 g, 2.44 mmol) in methanol (40.0 mL) was added several drops of thionyl chloride. The mixture was stirred at refluxing temperature for 72 h. Methanol was removed in vacuo and the residue was diluted with ether. The colorless solid was collected by filtration and dried in air to afford dimethyl 2,7-di-tert-butyl-9,9-dimethyl-9H-xanthene-4,5-dicarboxylate in 98% yield (1.05 g): ¹H NMR (300 MHz, DMSO-d₆) δ 7.68 (d, J=1.8 Hz, 2H), 7.46 (d, J=1.8 Hz, 2H), 3.81 (s, 6H), 1.61 (s, 6H), 1.27 (s, 18H).

### Preparation 11

### Preparation of (2,7-di-tert-butyl-9,9-dimethyl-9H-xanthene-4,5-diyl)dimethanol

To a stirred solution of dimethyl 2,7-di-tert-butyl-9,9-dimethyl-9H-xanthene-4,5-dicarboxylate (1.05 g, 2.40 mmol) in a mixture of ether/tetrahydrofuran (1/1, 40.0 mL) was added lithium aluminum hydride (0.22 g, 5.80 mmol). The mixture was stirred for 16 h at ambient temperature, followed by the addition of saturated sodium sulfate solution. The mixture was extracted with ethyl acetate. The organic solution was dried over anhydrous sodium sulfate, filtered and evaporated. The residue was diluted with ether and the colorless solid was collected by filtration and dried in air to afford (2,7-di-tert-butyl-9,9-dimethyl-9H-xanthene-4,5-diyl)dimethanol in 98% yield (0.91 g): ¹H NMR (300 MHz, DMSO-d₆) δ 7.33 (d, J=2.2 Hz, 2H), 7.30 (d, J=2.2 Hz, 2H), 4.62 (s, 4H), 1.55 (s, 6H), 1.26 (s, 18H).

### Preparation 12

### Preparation of 1,8-bis(bromomethyl)anthracene

To a stirred suspension of 1,8-bis(hydroxymethyl)anthracene (0.24 g, 1.00 mmol) in dry ether (30 mL) was added phosphorus tribromide (1.08 g, 4.00 mmol) in one portion. The mixture was stirred for 12 h at ambient temperature, washed with water, dried over sodium sulfate and filtered. The solvent was removed in vacuo and the residue was purified by flash chromatography (ethyl acetate/hexane 1/4) to afford 1,8-bis(bromomethyl)anthracene as a white solid in 96% yield (0.34 g): ¹H NMR (300 MHz, DMSO-d₆) δ 8.98 (s, 1H), 8.70 (s, 1H), 8.11 (d, J=8.5 Hz, 2H), 7.75 (dd, J=1.0, 6.8 Hz, 2H), 7.47 (dd, J=6.8, 8.5 Hz, 2H), 5.37 (s, 4H).

### Preparation 13

### Preparation of 4,6-bis(bromomethyl)dibenzo[b,d]furan

### A. Preparation of dibenzo[b,d]furan-4,6-diyldimethanol

To an ice-cold solution of dimethyl 2-iododibenzo[b,d]furan-4,6-dicarboxylate (12.00 g, 29.27 mmol) in a mixture of tetrahydrofuran/ether (1/1, 12 mL) was added lithium aluminum hydride in tetrahydrofuran (88 mL of 1 M solution, 88 mmol). The temperature was then raised to ambient temperature and stirred for 16 h under nitrogen. The reaction was quenched with saturated aqueous sodium sulfate (3 drops) and water (3 mL). The organic solvent was removed in vacuo and the residue was diluted with dichloromethane (300 mL) and filtered. The organic layer was concentrated in vacuo to afford dibenzo[b,d]furan-4,6-diyldimethanol in 3% yield (0.19 g): ¹H NMR (300 MHz, DMSO-d₆) δ 7.98 (d, J=7.7 Hz, 1H), 7.51 (d, J=7.4 Hz, 1H), 7.35 (d, J=7.7 Hz, 1H), 4.85 (s, 2H); MS (ES+) m/z 211.2 (M−17).

### B. Synthesis of 4,6-bis(bromomethyl)dibenzo[b,d]furan

Phosphorus tribromide (0.57 g, 21.72 mmol) was added to a mixture of dibenzo[b,d]furan-4,6-diyldimethanol (0.19 g, 0.83 mmol) in benzene (5 mL). The reaction mixture was stirred at ambient temperature for 3 h, followed by the addition of ice (3 g). The mixture was diluted with ethyl acetate (100 mL). The organic layer was dried over anhydrous sodium sulfate, filtered and concentrated in vacuo and the resulting residue was purified by column chromatography eluted with ethyl acetate/hexane (1/8) to afford 4,6-bis(bromomethyl)dibenzo[b,d]furan as a light yellow solid in 10% yield (0.03 g): ¹H NMR (300 MHz, CDCl₃) δ 7.90 (dd, J=7.7, 1.1 Hz, 1H), 7.52 (d, J=7.5 Hz, 1H), 7.35 (dd, J=7.7 Hz, 1H), 4.91 (s, 2H).

### Preparation 14

### Preparation of 3,7-dibromo-4,6-bis(bromomethyl)dibenzo[b,d]furan

To a stirred suspension of 3,7-dibromo-4,6-dimethyldibenzo[b,d]furan (0.24 g, 0.68 mmol) in carbon tetrachloride (50 mL) was added N-bromosuccinimide (0.24 g, 1.36 mmol) and benzoyl peroxide (5 mg) at ambient temperature. The mixture was stirred at 60° C. for 16 h, cooled to 0° C. and filtered. The filtrate was concentrated in vacuo. The residue was recrystallized from ethyl acetate/hexane to afford 3,7-dibromo-4,6-bis(bromomethyl)dibenzo[b,d]furan as a colorless solid in 40% yield (0.13 g): ¹H NMR (300 MHz, CDCl₃) δ 7.71 (d, J=8.3 Hz, 2H), 7.57 (d, J=8.3 Hz, 2H), 4.97 (s, 4H).

### Preparation 14.1

### Preparation of 3,7-dibromo-4,6-bis(bromomethyl)dibenzo[b,d]thiophene

Following the procedure as described in Preparation 14, making non-critical variation using 3,7-dibromo-4,6-dimethyldibenzo[b,d]thiophene to replace 3,7-dibromo-4,6-dimethyldibenzo[b,d]furan, 3,7-dibromo-4,6-bis(bromomethyl)dibenzo[b,d]thiophene was obtained as a colorless solid in 28% yield: ¹H NMR (300 MHz, CDCl₃) δ 7.89 (d, J=8.4 Hz, 2H), 7.70 (d, J=8.4 Hz, 2H), 4.90 (s, 4H).

### Preparation 14.2

### Preparation of 4,6-bis(bromomethyl)dibenzo[b,d]thiophene

Following the procedure as described in Preparation 14, making non-critical variation using 4,6-dimethyldibenzo[b,d]thiophene to replace 3,7-dibromo-4,6-dimethyldibenzo[b,d]furan, 4,6-bis(bromomethyl)dibenzo[b,d]thiophene was isolated as a white solid in 21% yield: ¹H NMR (300 MHz, DMSO-d₆) δ 8.10 (dd, J=1.4, 7.5 Hz, 2H), 7.51 (dd, J=1.4, 7.5 Hz, 2H), 7.47 (d, J=7.5 Hz, 2H), 4.79 (s, 4H).

### Preparation 14.3

### Preparation of 3-bromo-4,6-bis(bromomethyl)dibenzo[b,d]thiophene

Following the procedure as described in Preparation 14, making non-critical variation using 3-bromo-4,6-dimethyldibenzo[b,d]thiophene to replace 3,7-dibromo-4,6-dimethyldibenzo[b,d]furan, 3-bromo-4,6-bis(bromomethyl)dibenzo[b,d]thiophene was obtained as a colorless solid in 47% yield: ¹H NMR (300 MHz, CDCl₃) δ 8.12-8.04 (m, 1H), 7.94 (d, J=8.4 Hz, 1H), 7.69 (d, J=8.4 Hz, 1H), 7.58-7.44 (m, 2H), 4.93 (s, 2H), 4.79 (s, 2H).

### Preparation 14.4

### Preparation of 4,6-bis(bromomethyl)-3,7-difluorodibenzo[b,d]furan

Following the procedure as described in Preparation 14, making non-critical variation using 3,7-difluoro-4,6-dimethyldibenzo[b,d]furan to replace 3,7-dibromo-4,6-dimethyldibenzo[b,d]furan, 4,6-bis(bromomethyl)-3,7-difluorodibenzo[b,d]furan was obtained as a colorless solid in 38% yield: ¹H NMR (300 MHz, CDCl₃) δ 7.80 (dd, J=8.4, 5.1 Hz, 2H), 7.04 (dd, J=9.6, 8.7 Hz, 2H), 4.88 (s, 4H).

### Preparation 14.5

### Preparation of 4,6-bis(bromomethyl)-3,7-dichlorodibenzo[b,d]furan

Following the procedure as described in Preparation 14, making non-critical variation using 3,7-dichloro-4,6-dimethyldibenzo[b,d]furan to replace 3,7-dibromo-4,6-dimethyldibenzo[b,d]furan, 4,6-bis(bromomethyl)-3,7-dichlorodibenzo[b,d]furan was obtained as a colorless solid in 72% yield: ¹H NMR (300 MHz, CDCl₃) δ 7.78 (d, J=8.1 Hz, 2H), 7.42 (d, J=8.4 Hz, 2H), 4.98 (s, 4H).

### Preparation 14.6

### Preparation of 4,6-bis(bromomethyl)-2-fluorodibenzo[b,d]furan

Following the procedure as described in Preparation 14, making non-critical variation using 2-fluoro-4,6-dimethyldibenzo[b,d]furan to replace 3,7-dibromo-4,6-dimethyldibenzo[b,d]furan, 4,6-bis(bromomethyl)-2-fluorodibenzo[b,d]furan was obtained as a colorless solid in 32% yield: ¹H NMR (300 MHz, CDCl₃) δ 7.86 (d, J=7.8 Hz, 1H), 7.59-7.51 (m, 2H), 7.35 (dd, J=7.8, 7.5 Hz, 1H), 7.29-7.23 (m, 1H), 4.88 (s, 2H), 4.84 (s, 2H).

### Preparation 14.7

### Preparation of 4,6-bis(bromomethyl)dibenzo[b,d]furan

Following the procedure as described in Preparation 14, making non-critical variations using 4,6-dimethyldibenzo[b,d]furan to replace 3,7-dibromo-4,6-dimethyldibenzo[b,d]furan, 4,6-bis(bromomethyl)dibenzo[b,d]furan was obtained as a colorless solid in 35% yield: ¹H NMR (300 MHz, CDCl₃) δ 7.88 (dd, J=7.7, 0.8 Hz, 2H), 7.49 (d, J=7.7 Hz, 2H), 7.33 (t, J=7.7 Hz, 2H), 4.89 (s, 4H).

### Preparation 14.8

### Preparation of 1,9-bis(bromomethyl)dibenzo[b,d]thiophene

Following the procedure as described in Preparation 14, making non-critical variations using 1,9-dimethyldibenzo[b,d]thiophene (prepared according to Cho et al., J. Org. Chem., 2004, 69, 3811-3823) to replace 3,7-dibromo-4,6-dimethyldibenzo[b,d]furan, 1,9-bis(bromomethyl)dibenzo[b,d]thiophene was obtained as a colorless solid in 33% yield: ¹H NMR (300 MHz, CDCl₃) δ 7.78 (dd, J=7.8, 1.2 Hz, 2H), 7.69 (dd, J=7.8, 1.2 Hz, 2H), 7.49 (dd, J=7.7, 7.7 Hz, 2H), 5.08 (s, 4H).

### Preparation 14.9

### Preparation of 4,5-bis(bromomethyl)-9H-fluoren-9-one

Following the procedure described in Preparation 14, making non-critical variations using 4,5-dimethyl-9H-fluoren-9-one (Mulholland et al., J. Chem. Soc., 1956, 2415) to replace 3,7-dibromo-4,6-dimethyldibenzo[b,d]furan, 4,5-bis(bromomethyl)-9H-fluoren-9-one was obtained as a colorless solid in 57% yield: ¹H NMR (300 MHz, CDCl₃) δ 7.71 (dd, J=7.2, 1.2 Hz, 2H), 7.63 (dd, J=7.8, 1.2 Hz, 2H), 7.37 (dd, J=7.7, 7.7 Hz, 2H), 4.87 (s, 4H).

### Preparation 15

### Preparation of dibenzo[b,d]furan-4,6-dicarboxylic acid

N,N,N′,N′-Tetramethylethylenediamine (20.0 mL, 133.4 mmol) was added drop wise to a solution of dibenzo[b,d]furan (10.00 g, 59.50 mmol) in anhydrous ether (500 mL) under argon and the mixture was stirred at 0° C. for 30 min. s-Butyl lithium in cyclohexane (100 mL of 1.4 M solution, 140 mmol) was added to this cooled mixture dropwise. The reaction mixture was stirred at 25° C. for 18 h under argon and cooled to −78° C. Carbon dioxide (excess) was bubbled through the mixture for 3 h. The mixture was warmed to ambient temperature and stirred for 16 h. The solid precipitated upon slowly acidifying the reaction mixture with concentrated hydrochloric acid (to pH<2) was collected by filtration, washed with cold methanol (2×25 mL) and dried under high vacuum to afford dibenzo[b,d]furan-4,6-dicarboxylic acid as a colorless solid in 76% yield (11.5 g): ¹H NMR (300 MHz, DMSO-d₆) δ 8.44 (d, J=7.7 Hz, 1H), 8.03 (dd, J=7.6, 1.0 Hz, 1H), 7.51 (dd, J=7.8, 7.8 Hz, 1H); MS (ES−) m/z 255.2 (M−1).

### Preparation 16

### Preparation of N⁴,N⁴,N⁶,N⁶-tetraethyldibenzo[b,d]furan-4,6-dicarboxamide

A mixture of dibenzo[b,d]furan-4,6-dicarboxylic acid (1.05 g, 4.10 mmol) in trifluoroacetic acid (5.0 mL) and thionyl chloride (20 mL) was refluxed for 10 h under nitrogen. The reaction mixture was concentrated to dryness. N,N-Dimethylformamide (10 mL), triethylamine (1.0 mL), diethylamine (4.26 mL, 4.0 mol) were added to the residue and the resulting mixture was stirred under nitrogen for 16 h. The solvent was removed in vacuo and the residue was purified by column chromatography eluted with ethyl acetate/hexane (1/1) to afford N⁴,N⁴,N⁶,N⁶-tetraethyldibenzo[b,d]furan-4,6-dicarboxamide as a colorless solid in 86% yield (1.29 g): ¹H NMR (300 MHz, CDCl₃) δ 8.20-7.94 (m, 2H), 7.54-7.36 (m, 4H), 3.62 (q, J=6.6 Hz, 4H), 3.24 (q, J=7.2 Hz, 4H), 1.32 (t, J=7.2 Hz, 6H), 1.08 (t, J=7.2 Hz, 6H); MS (ES+) m/z 367.4 (M+1).

### Preparation 17

### Preparation of N⁴,N⁴,N⁶,N⁶-tetraethyl-3,7-dimethyldibenzo[b,d]furan-4,6-dicarboxamide

To a solution of N,N,N′,N′-tetramethylethylenediamine (1.32 mL, 8.80 mmol) in tetrahydrofuran (20 mL) at −78° C. were added s-butyl lithium in cyclohexane (6.29 mL of 1.4 M solution, 8.80 mmol) and N⁴,N⁴,N⁶,N⁶-tetraethyldibenzo[b,d]furan-4,6-dicarboxamide (1.47 g, 4.00 mmol). The reaction mixture was stirred at −78° C. for 1 h, followed by the addition of methyl iodide (0.75 mL, 12 mmol). The mixture was stirred at −78° C. for 3 h, followed by the addition of saturated ammonium chloride solution to quench the reaction, diluted with ethyl acetate (100 mL), and washed with water and brine. The organic layer was dried over anhydrous sodium sulfate, filtered and the filtrate was concentrated in vacuo to dryness. The residue was purified by column chromatography eluted with ethyl acetate/hexane (1/1) to afford N⁴,N⁴,N⁶,N⁶-tetraethyl-3,7-dimethyldibenzo[b,d]furan-4,6-dicarboxamide as a colorless solid in 40% yield (0.63 g): ¹H NMR (300 MHz, CDCl₃) δ 7.76 (d, J=7.8 Hz, 2H), 7.18 (d, J=7.8 Hz, 2H), 3.80-3.51 (m, 4H), 3.16 (q, J=7.2 Hz, 4H), 2.44 (s, 6H), 1.32 (t, J=7.2 Hz, 6H), 1.02 (t, J=7.2 Hz, 6H); MS (ES+) m/z 395.4 (M+1)

### Preparation 18

### Preparation of 3,7-dimethyldibenzo[b,d]furan-4,6-dicarbaldehyde

To a solution of N⁴,N⁴,N⁶, N⁶-tetraethyl-3,7-dimethyldibenzo[b,d]furan-4,6-dicarboxamide (0.50 g, 1.27 mmol) in tetrahydrofuran (20 mL) was added Shwartz reagent (0.98 g, 3.81 mmol). The reaction mixture was stirred at ambient temperature for 20 min, followed by the addition of water (2 mL). The mixture was diluted with ethyl acetate (100 mL), and washed with water and brine. The organic layer was dried over anhydrous sodium sulfate, filtered and the filtrate was concentrated in vacuo to dryness. The residue was purified by column chromatography eluted with ethyl acetate/hexane (1/3) to afford 3,7-dimethyldibenzo[b,d]furan-4,6-dicarbaldehyde as a colorless solid in 93% yield (0.30 g): ¹H NMR (300 MHz, CDCl₃) δ 10.96 (s, 2H), 8.01 (d, J=7.8 Hz, 2H), 7.28 (d, J=7.8 Hz, 2H), 2.82 (s, 6H); MS (ES+) m/z 253.2 (M+1).

### Preparation 19

### Preparation of 3,7-dimethyldibenzo[b,d]furan-4,6-diyl)dimethanol

To a solution of 3,7-dimethyldibenzo[b,d]furan-4,6-dicarbaldehyde (0.30 g, 1.19 mmol) in methanol (20 mL) was added sodium borohydride (0.14 g, 3.57 mmol) at ambient temperature. The reaction mixture was stirred at ambient temperature for 30 min, followed by the addition of 6 N hydrochloric acid solution (3 mL). The solvent was removed in vacuo. The residue was diluted with ethyl acetate (100 mL) and washed with water and brine. The organic layer was dried over anhydrous sodium sulfate, filtered and the filtrate was concentrated in vacuo to dryness. The residue was purified by column chromatography eluted with ethyl acetate/hexane (1/1) to afford 3,7-dimethyldibenzo[b,d]furan-4,6-diyl)dimethanol as a colorless solid in 57% yield (0.18 g): ¹H NMR (300 MHz, DMSO-d₆) δ 7.85 (d, J=7.8 Hz, 2H), 7.19 (d, J=7.8 Hz, 2H), 4.86 (s, 4H), 2.52 (s, 6H); MS (ES+) m/z 239.2 (M−17), 279.2 (M+23).

### Preparation 20

### Preparation of 4,6-bis(bromomethyl)-3,7-dimethyldibenzo[b,d]furan

To a solution of 3,7-dimethyldibenzo[b,d]furan-4,6-diyl)dimethanol (0.15 g, 0.57 mmol) in dichloromethane (10 mL) was added phosphorus tribromide (0.11 mL, 1.17 mmol) at ambient temperature. The reaction mixture was stirred at ambient temperature for 16 h, diluted with dichloromethane (100 mL), and washed with water and brine. The organic layer was dried over anhydrous sodium sulfate, filtered and the filtrate was concentrated in vacuo to dryness. The residue was purified by column chromatography eluted with ethyl acetate/hexane (1/1) to afford 4,6-bis(bromomethyl)-3,7-dimethyldibenzo[b,d]furan as a colorless solid in 56% (0.12 g): ¹H NMR (300 MHz, CDCl₃) δ 7.73 (d, J=7.8 Hz, 2H), 7.17 (d, J=7.8 Hz, 2H), 4.94 (s, 4H), 2.57 (s, 6H).

### Preparation 20.1

### Preparation of 2,8-dibromo-4,6-bis(bromomethyl)dibenzo[b,d]furan

Following the procedure as described in Preparation 20, making non-critical variations using 2,8-dibromodibenzo[b,d]furan-4,6-diyl)dimethanol to replace 3,7-dimethyldibenzo[b,d]furan-4,6-diyl)dimethanol, 2,8-dibromo-4,6-bis(bromomethyl)dibenzo[b,d]furan was obtained as a colorless solid in 30% yield: ¹H NMR (300 MHz, CDCl₃) δ 7.97 (d, J=1.8 Hz, 2H), 7.66 (d, J=1.8 Hz, 2H), 4.79 (s, 4H).

### Preparation 21

### Preparation of 2,8-dibromodibenzo[b,d]furan-4,6-diyl)dimethanol

### A. Preparation of dimethyl dibenzo[b,d]furan-4,6-dicarboxylate

To a mixture of dibenzo[b,d]furan-4,6-dicarboxylic acid (2.56 g, 10.00 mmol) in methanol (40 mL) was added thionyl chloride (1.0 mL, 2.0 mmol). The reaction mixture was refluxed for 4 h and poured into water (400 mL). The solid precipitated was collected by filtration, washed with water and hexane, and dried to afford dimethyl dibenzo[b,d]furan-4,6-dicarboxylate as a colorless solid in 89% yield (2.52 g): ¹H NMR (300 MHz, CDCl₃) δ 8.21-8.15 (m, 4H), 7.47 (dd, J=7.5, 7.5 Hz, 2H), 4.11 (s, 6H); MS (ES+) m/z 285.2 (M+1), 307.2 (M+23).

### B. Preparation of dimethyl 2,8-dibromodibenzo[b,d]furan-4,6-dicarboxylate

A mixture of dimethyl dibenzo[b,d]furan-4,6-dicarboxylate (0.50 g, 1.76 mmol), N-bromosuccinimide (0.94 g, 5.28 mmol) and iron(III) chloride (0.86 g, 5.28 mmol) in acetonitrile (30 mL) was heated to 130-140° C. for 16 h in a sealed tube, cooled to ambient temperature and poured into water (400 mL). The solid precipitated was collected by filtration, washed with water and hexane, and dried. The residue was recrystallized from ethyl acetate to afford dimethyl 2,8-dibromodibenzo[b,d]furan-4,6-dicarboxylate as a colorless solid in 73% yield (0.57 g): ¹H NMR (300 MHz, CDCl₃) δ 8.29 (d, J=1.8 Hz, 2H), 8.23 (d, J=1.8 Hz, 2H), 4.09 (s, 6H); MS (ES+) m/z 463.1 (M+23), 465.1 (M+23), 467.1 (M+23).

### C. Preparation of 2,8-dibromodibenzo[b,d]furan-4,6-diyl)dimethanol

To an ice cold mixture of dimethyl 2,8-dibromodibenzo[b,d]furan-4,6-dicarboxylate (0.50 g, 1.13 mmol) in chloroform (20 mL) was added lithium aluminum hydride (0.25 g, 6.58 mmol). The reaction mixture was stirred at ambient temperature for 1 h, then refluxed for 5 h. The reaction was quenched by slow addition of water. The mixture was diluted with chloroform (100 mL) and followed by the addition of concentrated hydrochloric acid (5 mL). The solid obtained was collected by filtration, washed with water and hexane, and dried to yield the first crop of crude product. The organic layer of the filtrate was separated, washed by water and brine, dried over anhydrous sodium sulfate, filtered and the filtrate was concentrated to yield another crop of crude product. The combined crude product was recrystallized from ether to afford 2,8-dibromodibenzo[b,d]furan-4,6-diyl)dimethanol as a colorless solid in 71% yield (0.31 g): ¹H NMR (300 MHz, CD₃OD) δ 8.07 (d, J=1.8 Hz, 2H), 7.67 (d, J=1.8 Hz, 2H), 4.95 (s, 4H).

### Preparation 22

### Preparation of (2,8-dibromodibenzo[b,d]furan-4,6-diyl)bis(methylene)bis(oxy)bis(tert-butyldimethylsilane)

To a mixture of 2,8-dibromodibenzo[b,d]furan-4,6-diyl)dimethanol (0.10 g, 0.26 mmol) in N,N-dimethylformamide (2 mL) were added imidazole (0.053 g, 0.78 mmol) and tert-butyldimethylsilyl chloride (0.12 g, 0.78 mmol). The reaction mixture was stirred at ambient temperature for 16 h and then poured into water (30 mL). The solid formed was collected by filtration, washed with water and hexane, and dried to afford (2,8-dibromodibenzo[b,d]furan-4,6-diyl)bis(methylene)bis(oxy)bis(tert-butyldimethylsilane) as a colorless solid: ¹H NMR (300 MHz, CDCl₃) δ 7.90 (s, 2H), 7.67 (s, 2H), 5.06 (s, 4H), 0.99 (s, 18H), 0.18 (s, 12H).

### Preparation 23

### Preparation of 4,5-bis(bromomethyl)-9-methylacridine

A solution of 9-methylacridine (2.16 g, 11.16 mmol) and bromo(methoxy)methane (6.08 g, 44.64 mmol) in concentrated sulfuric acid (25 mL) was stirred under nitrogen at 50° C. for 14 h. The reaction mixture was poured on ice and stirred for 1 h. The solid obtained was collected by filtration, then dissolved in chloroform. The resulting solution was dried over sodium sulfate and filtered. The filtrate was concentrated in vacuo and the residue was recrystallized from dichloroethane/hexane to afford 4,5-bis(bromomethyl)-9-methylacridine as an off-white solid in 40% yield (1.7 g): MS (ES+) m/z 378.1 (M+1), 380.3 (M+1).

### Preparation 24

### Preparation of 1,8-bis(bromomethyl)biphenylene

### A. Preparation of 1,8-dimethylbiphenylene

A mixture of 2,2′-diiodo-6,6′-dimethylbiphenyl (2.00 g, 4.61 mmol) and finely divided copper (2.00 g, 31.47 mmol) was heated at 250-270° C. for 1.5 h with occasional mechanical stirring. The reaction mixture was allowed to cool to ambient temperature and the residue was extracted with boiling acetone (5×10 mL). The combined extracts were filtered through a pad of diatomaceous earth and the pad was washed with acetone (50 mL). The filtrate was concentrated in vacuo and the residue was purified by column chromatography eluted with hexanes to afford 1,8-dimethylbiphenylene as a pale yellow solid in 60% yield (0.50 g): ¹H NMR (300 MHz, CDCl₃) δ 6.67-6.60 (m, 2H), 6.53 (d, J=8.2 Hz, 2H), 6.45 (d, J=8.2 Hz, 2H), 2.18 (s, 6H).

### B. Preparation of 1,8-bis(bromomethyl)biphenylene

To a solution of 1,8-dimethylbiphenylene (0.50 g, 2.77 mmol) in anhydrous carbon tetrachloride (20 mL) was added N-bromosuccinimide (0.99 g, 5.5 mmol) and benzoyl peroxide (0.03 g, 0.12 mmol). The reaction mixture was heated at reflux for 4 h and, while still hot, was filtered to remove precipitated succinimide. The filtrate was concentrated in vacuo to a volume of 5 mL and hexanes (50 mL) was added, causing a precipitate to be deposited. The solid was collected by suction filtration, washed with hexanes (20 mL) and air-dried to afford 1,8-bis(bromomethyl)biphenylene as a yellow solid in 51% yield (0.48 g): ¹H NMR (300 MHz, DMSO-d₆) δ 6.82-6.70 (m, 4H), 6.58 (dd, J=6.6, 0.8 Hz, 2H), 4.43 (s, 4H).

### Preparation 25

### Preparation of dimethyl 3,6-difluorobiphenylene-1,8-dicarboxylate

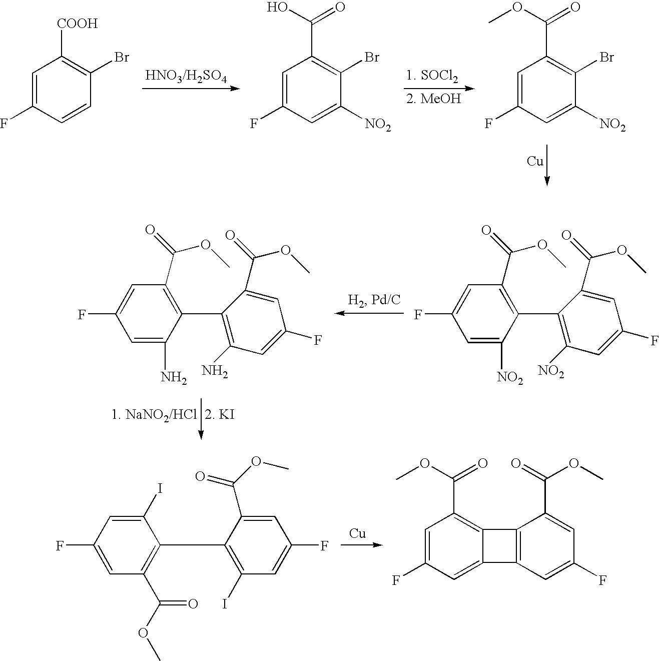

A. 2-Bromo-5-fluorobenzoic acid (44.00 g, 200.00 mmol) was dissolved in a mixture of concentrated sulfuric acid (350 mL) and fuming sulfuric acid (10 mL, 20% SO₃). To the above solution was added 90% nitric acid (30 mL) at 15-25° C. The reaction mixture was stirred at ambient temperature for 1 hour and poured in ice (1 Kg). The solid residue was collected, washed with water and dried. The filtrate was extracted with ethyl acetate (1 L). The extract was dried over magnesium sulfate, filtered and concentrated. The residue was purified by column chromatography and the solid obtained after removal of solvent was combined with the solid obtained earlier to afford 2-bromo-5-fluoro-3-nitrobenzoic acid in 28% yield (14.50 g): ¹H NMR (300 MHz, DMSO-d₆) δ 8.20 (dd, J=7.8, 3.0 Hz, 1H), 7.89 (dd, J=8.3, 3.0 Hz, 1H).

B. To a suspension of 2-bromo-5-fluoro-3-nitrobenzoic acid (34.00 g, 120.00 mmol) in dichloromethane (100 mL) was added thionyl chloride (50.00 g, 420.00 mmol) and dimethylformamide (5 mL). The reaction mixture was stirred to 40° C. for 20 hours. The solvent and excess of thionyl chloride were removed under reduced pressure and the residue was dried in vacuo for 20 hours and then dissolved in dichloromethane (100 mL). The resulting solution was added to methanol (100 mL) and the mixture was stirred at 40° C. for 30 minutes. The solvent was removed in vacuo and the residue was purified by column chromatography to afford methyl 2-bromo-5-fluoro-3-nitrobenzoate in 64% yield (21.4 g): ¹H NMR (300 MHz, CDCl₃) δ 7.61 (dd, J=7.8, 3.0 Hz, 1H), 7.52 (dd, J=6.9, 3.0 Hz, 1H), 3.96 (s, 3H).

C. To a solution of methyl 2-bromo-5-fluoro-3-nitrobenzoate (21.40 g, 77.00 mmol) in dimethylformamide (100 mL) was added activated copper powder (15.00 g, 240.00 mmol). The reaction mixture was warmed up to reflux for 1.5 hour, cooled to ambient temperature, and filtered. The filtrate was poured into water (1 L). The water solution was extracted with ethyl acetate (3×500 mL) and the combined extract was dried over magnesium sulfate, filtered and concentrated. The residue was purified by column chromatography to afford dimethyl 4,4′-difluoro-6,6′-dinitrobiphenyl-2,2′-dicarboxylate in 81% yield (11.80 g): ¹H NMR (300 MHz, CDCl₃) δ 8.08-8.01 (m, 4H), 3.68 (s, 6H).

D. To a solution of 4,4′-difluoro-6,6′-dinitrobiphenyl-2,2′-dicarboxylate (11.80 g, 30.00 mmol) in ethyl acetate (150 mL) was added palladium on carbon (3.0 g, 20%). The mixture was hydrogenated in Parr hydrogenator at 35-40 psi for 16 hours and filtered to remove the catalyst. The filtrate was concentrated and the residue was purified by column chromatography to afford dimethyl 6,6′-diamino-4,4′-difluorobiphenyl-2,2′-dicarboxylate in 73% yield (7.40 g): ¹H NMR (300 MHz, CDCl₃) δ 6.76 (dd, J=9.4, 2.7 Hz, 2H), 6.66 (dd, J=11.1, 2.7 Hz, 2H), 4.81 (s, 4H), 3.46 (s, 6H).

E. To a suspension of dimethyl 6,6′-diamino-4,4′-difluorobiphenyl-2,2′-dicarboxylate (6.20 g, 18.00 mmol) in trifluoroacetic acid (40 mL) was added concentrated hydrochloric acid (15 mL). The reaction mixture was cooled down to 5° C. and a solution of sodium nitrite (2.64 g, 38.00 mmol) in water (10 mL) was added in small portions and the reaction mixture was kept 8° C. during this process. After 30 minutes a solution of urea (2.0 g) in water (10 mL) was added. The mixture was kept at 5° C. for another 30 minutes and added into a solution of potassium iodide (12.61 g, 76.00 mmol) in water (50 mL) and ice (50 g). The mixture was stirred for 30 minutes at 5° C. and extracted with ethyl acetate (2×100 mL). The combined extract was washed with saturated sodium bicarbonate, dried over magnesium sulfate, filtered and concentrated. The residue was purified by column chromatography to afford dimethyl 4,4′-difluoro-6,6′-diiodobiphenyl-2,2′-dicarboxylate in 30% yield (3.01 g): ¹H NMR (300 MHz, CDCl₃) δ 7.87-7.79 (m, 4H), 3.67 (d, J=1.1 Hz, 6H).

F. To a solution of dimethyl 4,4′-difluoro-6,6′-diiodobiphenyl-2,2′-dicarboxylate (1.0 g, 1.79 mmol) in dimethylformamide (1.0 mL) was added copper powder (1.00 g, 15.70 mmol). The reaction mixture was warmed up to 230° C. for 15 minutes in a microwave reactor (100 W) and the residue was purified by column chromatography to afford dimethyl 3,6-difluorobiphenylene-1,8-dicarboxylate in 12% yield (0.075 g). MS (ES+) m/z 305.1 (M+1).

### Example 1

### Synthesis of dibenzo[b,d]thiophene-4,6-diylbis(methylene) dicarbamimidothioate dihydrobromide

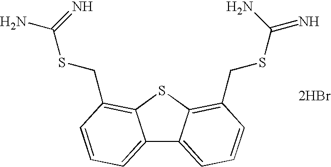

To the solution of 4,6-bis(bromomethyl)dibenzo[b,d]thiophene (0.19 g, 0.50 mmol) in ethanol (10 mL) was added thiourea (0.076 g, 1.0 mmol). The mixture was maintained at 80° C. for 14 h and cooled to ambient temperature. Ethanol was removed to one third of the initial volume and hexane was added. The solid precipitated was collected by filtration, washed with ether and ethyl acetate and dried in air to afford dibenzo[b,d]thiophene-4,6-diylbis(methylene) dicarbamimidothioate dihydrobromide in 84% yield (0.22 g): ¹H NMR (300 MHz, DMSO-d₆) δ 9.34 (br s, 4H), 9.13 (br s, 4H), 8.40 (d, J=7.7 Hz, 2H), 7.64 (d, J=7.7 Hz, 2H), 7.57 (dd, J=7.7, 7.7 Hz, 2H), 4.81 (s, 4H); ¹³C NMR (75 MHz, DMSO-d₆) δ 169.3, 138.3, 136.6, 129.1, 128.9, 126.3, 123.0, 34.5; MS (ES+) m/z 361.1 (M+1).

### Example 1.1

### Synthesis of (3,7-dibromodibenzo[b,d]thiophene-4,6-diyl)bis(methylene) dicarbamimidothioate dihydrobromide

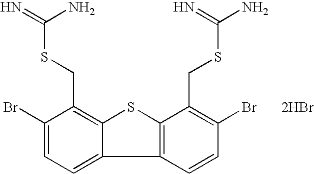

Following the procedure as described in Example 1, making non-critical variations using 3,7-dibromo-4,6-bis(bromomethyl)dibenzo[b,d]thiophene to replace 4,6-bis(bromomethyl)dibenzo[b,d]thiophene, (3,7-dibromodibenzo[b,d]thiophene-4,6-diyl)bis(methylene) dicarbamimidothioate dihydrobromide was obtained as a colorless solid in 85% yield: mp>220° C.; ¹H NMR (300 MHz, DMSO-d₆) δ 9.62-9.16 (br s, 8H), 8.37 (d, J=8.4 Hz, 2H), 7.89 (d, J=8.4 Hz, 2H), 4.90 (s, 4H); ¹³C NMR (75 MHz, DMSO-d₆) δ 169.4, 140.8, 135.5, 131.0, 127.0, 125.0, 124.2, 36.0; MS (ES+) m/z 517.1 (M+1), 519.1 (M+1), 521.1 (M+1).

### Example 1.2

### Synthesis of (3-bromodibenzo[b,d]thiophene-4,6-diyl)bis(methylene) dicarbamimidothioate dihydrobromide

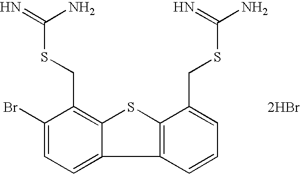

Following the procedure as described in Example 1, making non-critical variations using 3-bromo-4,6-bis(bromomethyl)dibenzo[b,d]thiophene to replace 4,6-bis(bromomethyl)dibenzo[b,d]thiophene, (3-bromodibenzo[b,d]thiophene-4,6-diyl)bis(methylene) dicarbamimidothioate dihydrobromide was obtained as a colorless solid in 51% yield: mp>220° C.; ¹H NMR (300 MHz, DMSO-d₆) δ 9.51-9.10 (br s, 8H), 8.46 (d, J=7.8 Hz, 1H), 8.40 (d, J=8.4 Hz, 1H), 7.91 (d, J=7.8 Hz, 1H), 7.72-7.59 (m, 2H), 4.89 (s, 4H); ¹³C NMR (75 MHz, DMSO-d₆) δ 169.0, 168.6, 140.3, 137.8, 135.7, 135.5, 130.2, 128.9, 128.7, 126.4, 126.2, 124.4, 123.2, 122.8, 35.6, 33.8; MS (ES+) m/z 439.2 (M+1), 441.2 (M+1).

### Example 1.3

### Synthesis of (3,7-dimethyldibenzo[b,d]furan-4,6-diyl)bis(methylene) dicarbamimidothioate dihydrobromide

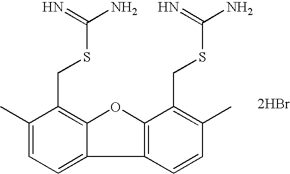

Following the procedure as described in Example 1, making non-critical variations using 4,6-bis(bromomethyl)-3,7-dimethyldibenzo[b,d]furan to replace 4,6-bis(bromomethyl)dibenzo[b,d]thiophene, (3,7-dimethyldibenzo[b,d]furan-4,6-diyl)bis(methylene) dicarbamimidothioate dihydrobromide was obtained as a colorless solid in 91% yield: mp>220° C.; ¹H NMR (300 MHz, DMSO-d₆) δ 9.50-9.00 (br s, 8H), 7.99 (d, J=7.8 Hz, 2H), 7.32 (d, J=7.8 Hz, 2H), 4.93 (s, 4H), 2.55 (s, 6H); ¹³C NMR (75 MHz, DMSO-d₆) δ 169.1, 154.3, 136.9, 125.9, 121.9, 120.9, 116.2, 27.4, 18.7; MS (ES+) m/z 373.2 (M+1).

### Example 1.4

### Synthesis of (3,7-difluorodibenzo[b,d]furan-4,6-diyl)bis(methylene) dicarbamimidothioate dihydrobromide

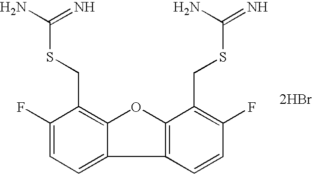

Following the procedure as described in Example 1, making non-critical variations using 4,6-bis(bromomethyl)-3,7-difluorodibenzo[b,d]furan to replace 4,6-bis(bromomethyl)dibenzo[b,d]thiophene, (3,7-difluorodibenzo[b,d]furan-4,6-diyl)bis(methylene) dicarbamimidothioate dihydrobromide was obtained as a colorless solid in 88% yield: mp>220° C.; ¹H NMR (300 MHz, DMSO-d₆) δ 9.50-9.01 (br s, 8H), 8.22 (dd, J=8.7, 5.1 Hz, 2H), 7.43 (dd, J=10.2, 8.7 Hz, 2H), 4.92 (s, 4H); ¹³C NMR (75 MHz, DMSO-d₆) δ 168.3, 161.0, 157.8, 122.2 (d, J_{C—F}=10.8 Hz), 119.8, 111.9 (d, J_{C—F}=23.4 Hz), 107.9 (d, J_{C—F}=21.2 Hz), 24.0; MS (ES+) m/z 381.1 (M+1).

### Example 1.5

### Synthesis of (3,7-dichlorodibenzo[b,d]furan-4,6-diyl)bis(methylene) dicarbamimidothioate

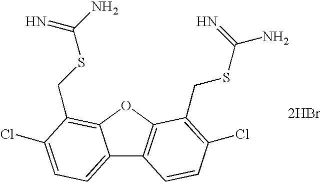

Following the procedure as described in Example 1, making non-critical variations using 4,6-bis(bromomethyl)-3,7-dichlorodibenzo[b,d]furan to replace 4,6-bis(bromomethyl)dibenzo[b,d]thiophene, (3,7-dichlorodibenzo[b,d]furan-4,6-diyl)bis(methylene) dicarbamimidothioate was obtained as a colorless solid in 93% yield: mp>220° C.; ¹H NMR (300 MHz, DMSO-d₆) δ 9.56-9.00 (br s, 8H), 8.24 (d, J=8.4 Hz, 2H), 7.32 (d, J=8.4 Hz, 2H), 4.98 (s, 4H); ¹³C NMR (75 MHz, DMSO-d₆) δ 168.9, 154.9, 133.2, 125.9, 123.2, 122.9, 118.1, 28.3; MS (ES+) m/z 413 (M+1), 415 (M+1).

### Example 1.6

### Synthesis of (2-fluorodibenzo[b,d]furan-4,6-diyl)bis(methylene) dicarbamimidothioate dihydrobromide

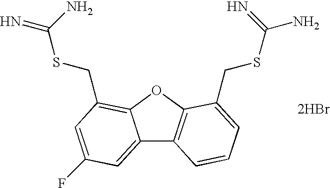

Following the procedure as described in Example 1, making non-critical variations using 4,6-bis(bromomethyl)-2-fluorodibenzo[b,d]furan to replace 4,6-bis(bromomethyl)dibenzo[b,d]thiophene, (2-fluorodibenzo[b,d]furan-4,6-diyl)bis(methylene) dicarbamimidothioate dihydrobromide was obtained as a colorless solid in 71% yield: mp>220° C.; ¹H NMR (300 MHz, DMSO-d₆) δ 9.52-9.00 (br s, 8H), 8.17 (d, J=7.5 Hz, 1H), 8.10 (dd, J=8.1, 2.4 Hz, 1H), 7.68-7.43 (m, 3H), 4.88 (s, 2H), 4.87 (s, 2H); MS (ES+) m/z 363.2 (M+1).

### Example 1.7

### Synthesis of (2,8-dibromodibenzo[b,d]furan-4,6-diyl)bis(methylene) dicarbamimidothioate dihydrobromide

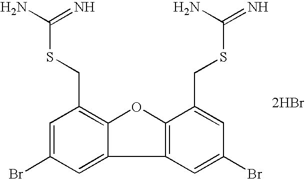

Following the procedure as described in Example 1, making non-critical variations using 2,8-dibromo-4,6-bis(bromomethyl)dibenzo[b,d]furan to replace 4,6-bis(bromomethyl)dibenzo[b,d]thiophene, (2,8-dibromodibenzo[b,d]furan-4,6-diyl)bis(methylene) dicarbamimidothioate dihydrobromide was obtained as a colorless solid in 92% yield: mp>220° C.; ¹H NMR (300 MHz, DMSO-d₆) δ 9.50-9.00 (br s, 8H), 8.51 (d, J=2.1 Hz, 2H), 7.84 (d, J=2.1 Hz, 2H), 4.84 (s, 4H); ¹³C NMR (75 MHz, DMSO-d₆) δ 168.8, 153.1, 131.6, 125.5, 125.1, 122.8, 116.2, 29.0; MS (ES+) m/z 500.9 (M+1), 502.9 (M+1), 504.9 (M+1).

### Example 1.8

### Synthesis of dibenzo[b,d]furan-4,6-diylbis(methylene) dicarbamimidothioate dihydrobromide

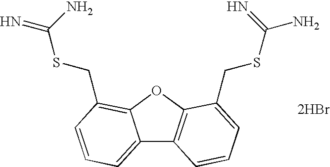

Following the procedure as described in Example 1, making non-critical variations using 4,6-bis(bromomethyl)dibenzo[b,d]furan to replace 4,6-bis(bromomethyl)dibenzo[b,d]thiophene, dibenzo[b,d]furan-4,6-diylbis(methylene) dicarbamimidothioate dihydrobromide was obtained as a colorless solid in 93% yield: mp>250° C.; ¹H NMR (300 MHz, DMSO-d₆) δ 9.18 (br s, 4H), 8.13 (d, J=7.7 Hz, 1H), 7.58 (d, J=7.5 Hz, 1H), 7.42 (t, dd=7.5, 7.5 Hz, 1H), 4.86 (s, 2H); ¹³C NMR (75 MHz, DMSO-d₆) δ 169.3, 153.8, 128.7, 124.34, 124.3, 122.0, 119.9, 29.5; MS (ES+) m/z 345.3 (M+1).

### Example 1.9

### Synthesis of anthracene-1,8-diylbis(methylene) dicarbamimidothioate dihydrobromide

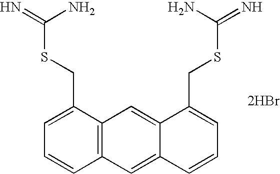

Following the procedure as described in Example 1, making non-critical variations using 1,8-bis(bromomethyl)anthracene to replace 4,6-bis(bromomethyl)dibenzo[b,d]thiophene, anthracene-1,8-diylbis(methylene) dicarbamimidothioate dihydrobromide was obtained as a colorless solid in 95% yield: mp>250° C.; ¹H NMR (300 MHz, DMSO-d₆) δ 9.24 (br s, 4H), 9.08 (br s, 4H), 8.89 (s, 1H), 8.70 (s, 1H), 8.09 (d, J=8.5 Hz, 2H), 7.68 (d, J=6.6 Hz, 2H), 7.51 (dd, J=6.6, 8.5 Hz, 2H), 5.26 (s, 4H); ¹³C NMR (75 MHz, DMSO-d₆) δ 169.6, 131.9, 131.5, 129.6, 129.3, 129.0, 128.5, 125.8, 119.7, 33.7; MS (ES+) m/z 355.1 (M+1).

### Example 1.10

### Synthesis of (2-fluorodibenzo[b,d]thiophene-4,6-diyl)bis(methylene) dicarbamimidothioate dihydrobromide

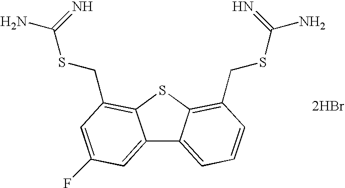

Following the procedure as described in Example 1, making non-critical variations using 4,6-bis-bromomethyl-2-fluoro-dibenzothiophene to replace 4,6-bis(bromomethyl)dibenzo[b,d]thiophene, (2-fluorodibenzo[b,d]thiophene-4,6-diyl)bis(methylene) dicarbamimidothioate dihydrobromide was obtained as a colorless solid in 84% yield: mp>230° C.; ¹H NMR (300 MHz, DMSO-d₆) δ 9.31 (br s, 4H), 9.13 (br s, 4H), 8.43 (d, J=7.0 Hz, 1H), 8.36 (dd, J=9.3, 2.5 Hz, 1H), 7.68-7.53 (m, 3H), 4.80 (s, 4H); MS (ES+) m/z 379.1 (M+1).

### Example 1.11

### Synthesis of (2-chloro-8-fluorodibenzo[b,d]thiophene-4,6-diyl)bis(methylene) dicarbamimidothioate dihydrobromide

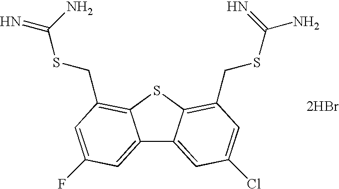

Following the procedure as described in Example 1, making non-critical variations using 4,6-bis(bromomethyl)-2-chloro-8-fluorodibenzo[b,d]thiophene to replace 4,6-bis(bromomethyl)dibenzo[b,d]thiophene, (2-chloro-8-fluorodibenzo[b,d]thiophene-4,6-diyl)bis(methylene) dicarbamimidothioate dihydrobromide was obtained as a colorless solid in 95% yield: ¹H NMR (300 MHz, DMSO-d₆) δ 9.32 (br s, 4H), 9.13 (br s, 4H), 8.62 (d, J=2.0 Hz, 1H), 8.43 (dd, J=2.5, 9.3 Hz, 1H), 7.75 (d, J=2.0 Hz, 1H), 7.61 (dd, J=2.5, 9.3 Hz, 1H), 4.81 (s, 4H); ¹³C NMR (75 MHz, DMSO-d₆) δ 168.8 (2C), 160.9 (d, J=241.9 Hz, 1C), 137.8, 137.4 (d, J=3.9 Hz, 1C), 136.8 (d, J=9.9 Hz, 1C), 134.4 (d, J=1.5 Hz, 1C), 131.6, 131.4 130.9, 128.8, 123.1, 117.4 (d, J=25.7 Hz, 1C), 109.8 (d, J=23.9 Hz, 1C), 33.9, 33.8; MS (ES+) m/z 413.1 (M+1).

### Example 1.12

### Synthesis of phenoxathiine-4,6-diylbis(methylene) dicarbamimidothioate dihydrobromide

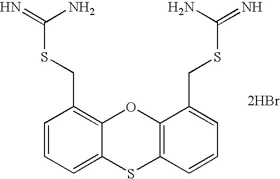

Following the procedure as described in Example 1, making non-critical variations using 4,6-bisbromomethylphenoxathiine to replace 4,6-bis(bromomethyl)dibenzo[b,d]thiophene, phenoxathiine-4,6-diylbis(methylene) dicarbamimidothioate dihydrobromide was obtained as a colorless solid in 93% yield: mp>230° C.; ¹H NMR (300 MHz, DMSO-d₆) δ 9.17 (br s, 8H), 7.31 (dd, J=1.4, 7.7 Hz, 2H), 7.26 (dd, J=1.4, 7.7 Hz, 2H), 7.12 (dd, J=7.7, 7.7 Hz, 2H), 4.61 (s, 4H); ¹H NMR (300 MHz, DMSO-d₆) δ 169.2, 149.3, 129.6, 127.6, 125.7, 124.5, 119.7, 30.3; MS (ES+) m/z 377.1 (M+1).

### Example 1.13

### Synthesis of (3,7-dibromodibenzo[b,d]furan-4,6-diyl)bis(methylene) dicarbamimidothioate dihydrobromide

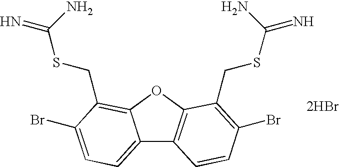

Following the procedure as described in Example 1, making non-critical variations using 3,7-dibromo-4,6-bis(bromomethyl)dibenzo[b,d]furan to replace 4,6-bis(bromomethyl)dibenzo[b,d]thiophene, (3,7-dibromodibenzo[b,d]furan-4,6-diyl)bis(methylene) dicarbamimidothioate dihydrobromide was obtained as a colorless solid in 79% yield: mp>250° C.; ¹H NMR (300 MHz, DMSO-d₆) δ 9.50-9.00 (br s, 8H), 8.14 (d, J=8.2 Hz, 2H), 7.76 (d, J=8.2 Hz, 1H), 4.93 (s, 4H); ¹³C NMR (75 MHz, DMSO-d₆) δ 169.0, 154.7, 129.0, 123.8, 123.6, 123.5, 119.5, 30.6; MS (ES+) m/z 501.2 (M+1), 503.2 (M+1), 505.2 (M+1).

### Example 1.14

### Synthesis of dibenzo[b,d]thiophene-1,9-diylbis(methylene) dicarbamimidothioate dihydrobromide

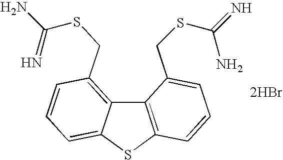

Following the procedure as described in Example 1, making non-critical variations using 1,9-bis(bromomethyl)dibenzo[b,d]thiophene to replace 4,6-bis(bromomethyl)dibenzo[b,d]thiophene, dibenzo[b,d]thiophene-1,9-diylbis(methylene) dicarbamimidothioate dihydrobromide was obtained as a colorless solid in 98% yield: mp 228-230° C.; ¹H NMR (300 MHz, DMSO-d₆) δ 9.23-8.88 (br s, 8H), 8.02 (dd, J=7.6, 1.2 Hz, 2H), 7.69-7.56 (m, 4H), 4.97 (s, 4H); ¹³C NMR (75 MHz, DMSO-d₆) δ 169.2, 140.5, 133.3, 131.6, 127.9, 127.8, 123.2, 36.9; MS (ES+) m/z 361.1 (M+1).

### Example 1.15

### Synthesis of (9-oxo-9H-fluorene-4,5-diyl)bis(methylene) dicarbamimidothioate dihydrobromide

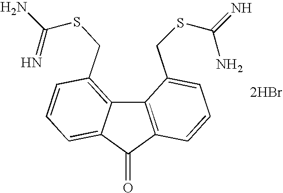

Following the procedure as described in Example 1, making non-critical variations using 4,5-bis(bromomethyl)-9H-fluoren-9-one to replace 4,6-bis(bromomethyl)dibenzo[b,d]thiophene, (9-oxo-9H-fluorene-4,5-diyl)bis(methylene) dicarbamimidothioate dihydrobromide was obtained as a colorless solid in 91% yield: mp 218-220° C.; ¹H NMR (300 MHz, DMSO-d₆) δ 9.33-8.93 (br s, 8H), 7.76 (dd, J=7.6, 1.2 Hz, 2H), 7.65 (dd, J=7.6, 1.2 Hz, 2H), 7.49 (dd, J=7.6, 7.6 Hz, 2H), 4.82 (s, 4H); ¹³C NMR (75 MHz, DMSO-d₆) δ 191.8, 168.8, 144.1, 138.1, 136.0, 131.0, 130.7, 124.2, 35.5; MS (ES+) m/z 357.2 (M+1).

### Example 1.16

### Synthesis of (9-oxo-9H-xanthene-4,5-diyl)bis(methylene) dicarbamimidothioate dihydrobromide

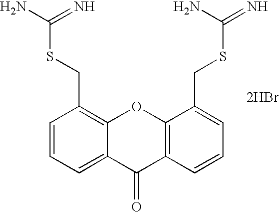

Following the procedure as described in Example 1, making non-critical variations using 4,5-bis(bromomethyl)-9H-xanthen-9-one (prepared according to Atwell et. al., J. Med. Chem., 1990, 33, 1375-1379) to replace 4,6-bis(bromomethyl)-dibenzo[b,d]thiophene, (9-oxo-9H-xanthene-4,5-diyl)bis(methylene) dicarbamimidothioate dihydrobromide was obtained as a colorless solid in 90% yield: mp>230° C.; ¹H NMR (300 MHz, DMSO-d₆) δ 9.35-9.00 (br, 8H), 8.20-8.15 (m, 2H), 7.99-7.94 (m, 2H), 7.51 (dd, J=7.8, 7.8 Hz, 2H), 4.89 (s, 4H); ¹³C NMR (75 MHz, DMSO-d₆) δ176.0, 169.0, 153.5, 136.5, 126.8, 125.1, 125.0, 121.8, 29.9; MS (ES+) m/z 373.1 (M+1).

### Example 1.17

### Synthesis of (9-methylacridine-4,5-diyl)bis(methylene) dicarbamimidothioate dihydrobromide

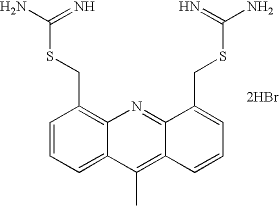

Following the procedure as described in Example 1, making non-critical variations using 4,5-bis(bromomethyl)-9-methylacridine to replace 4,6-bis(bromomethyl)-dibenzo[b,d]thiophene, (9-methylacridine-4,5-diyl)bis(methylene) dicarbamimidothioate dihydrobromide was obtained as a pale yellow solid in 74% yield: mp>270° C. (dec.); ¹H NMR (300 MHz, DMSO-d₆) δ 9.14 (br s, 8H), 8.42-8.37 (m, 2H), 8.05-8.01 (m, 2H), 7.66-7.59 (m, 2H), 5.15 (s, 4H), 3.10 (s, 3H); ¹³C NMR (75 MHz, DMSO-d₆) δ 170.6, 145.6, 144.9, 134.0, 131.5, 126.4, 126.0, 125.5, 32.1, 14.4; MS (ES+) m/z 370.2 (M+1).

### Example 1.18

### Synthesis of acridine-4,5-diylbis(methylene) dicarbamimidothioate dihydrobromide

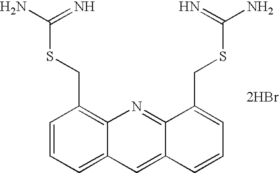

Following the procedure as described in Example 1, making non-critical variations using 4,5-bis(bromomethyl)acridine (prepared according to Giorgio et al., Bioorg. Med. Chem., 2005, 13, 5560-5568) to replace 4,6-bis(bromomethyl)dibenzo[b,d]thiophene, acridine-4,5-diylbis(methylene) dicarbamimidothioate dihydrobromide was obtained as a pale yellow solid in 84% yield: mp>270° C. (dec.); ¹H NMR (300 MHz, DMSO-d₆) δ 9.23 (br s, 4H), 9.20 (s, 1H), 9.15 (br s, 4H), 8.20-8.15 (m, 2H), 8.06-8.03 (m, 2H), 7.69-7.65 (m, 2H), 5.16 (s, 4H); ¹³C NMR (75 MHz, DMSO-d₆) δ 170.5, 145.6, 138.5, 133.5, 131.9, 129.7, 126.7, 126.4, 31.8; MS (ES+) m/z 356.2 (M+1).

### Example 1.19

### Synthesis of (5,7-dihydrodibenzo[c,e]thiepine-1,1′-diyl)bis(methylene) dicarbamimidothioate dihydrobromide

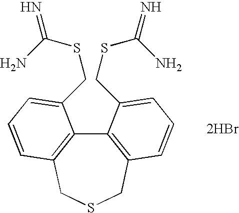

Following the procedure as described in Example 1, making non-critical variations using 1,11-bis(bromomethyl)-5,7-dihydrodibenzo[c,e]thiepine (prepared according to Mislow, et al., J. Am. Chem. Soc. 1964, 86(9):1710-1733) to replace 4,6-bis(bromomethyl)dibenzo[b,d]thiophene, (5,7-dihydrodibenzo[c,e]thiepine-1,11-diyl)bis(methylene) dicarbamimidothioate dihydrobromide was obtained as a colorless solid in 95% yield: mp 155-158° C. (hexanes); ¹H NMR (300 MHz, DMSO-d₆) δ 9.06 (br s, 4H), 8.89 (br s, 4H), 7.57 (dd, J=7.5, 1.2 Hz, 2H), 7.48 (dd, J=7.5, 7.5 Hz, 2H), 7.43 (dd, J=7.5, 1.2 Hz, 2H), 4.54 (d, J=12.8 Hz, 2H), 3.99 (d, J=12.8 Hz, 2H), 3.57 (d, J=12.5 Hz, 2H), 2.93 (d, J=12.5 Hz, 2H); ¹³C NMR (75 MHz, DMSO-d₆) δ 168.6, 136.9, 135.6, 131.8, 129.8, 129.5, 128.2, 33.6, 31.0; MS (ES+) m/z 389.1 (M+1).

### Example 2

### Synthesis of (9-oxo-9H-fluorene-1,8-diyl)bis(methylene) dicarbamimidothioate dihydrobromide

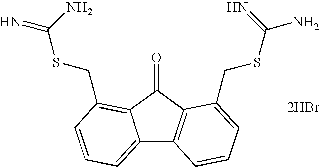

A mixture of 1,8-bis(bromomethyl)-9H-fluoren-9-one (0.22 g, 0.60 mmol), thiourea (0.09 g, 1.20 mmol) and anhydrous ethanol (4.0 mL) was heated in a sealed tube under microwave irradiation (80 W, 100° C.) for 10 min. The reaction mixture was allowed to cool to ambient temperature and the product was collected by filtration, washed with ice-cold ethanol (5 mL), air-dried and dried under high vacuum to obtain (9-oxo-9H-fluorene-1,8-diyl)bis(methylene) dicarbamimidothioate dihydrobromide as a yellow solid in 51% yield (0.16 g): mp>250° C. (ethanol); ¹H NMR (300 MHz, DMSO-d₆) δ 9.35-9.02 (m, 8H), 7.85 (d, J=7.4 Hz, 2H), 7.62 (dd, J=7.4, 7.4 Hz, 2H), 7.42 (d, J=7.4 Hz, 2H), 4.76 (s, 4H); ¹³C NMR (75 MHz, DMSO-d₆) δ 194.1, 169.0, 144.3, 135.8, 134.9, 131.2, 129.5, 121.5, 29.7; MS (ES−) m/z 516.9 (M−1).

### Example 2.1

### Synthesis of biphenylene-1,8-diylbis(methylene)dicarbamimidothioate dihydrobromide

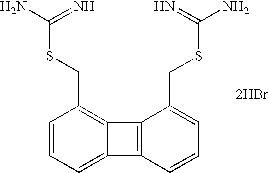

Following the procedure as described in Example 2, making non-critical variations using 1,8-bis(bromomethyl)biphenylene to replace 1,8-bis(bromomethyl)-9H-fluoren-9-one, biphenylene-1,8-diylbis(methylene) dicarbamimidothioate dihydrobromide was obtained as a yellow solid in 64% yield: mp>250° C. (ethanol); ¹H NMR (300 MHz, DMSO-d₆) δ 9.35-9.08 (m, 8H), 6.90-6.79 (m, 4H), 6.77-6.72 (m, 2H), 4.45 (s, 4H); ¹³C NMR (75 MHz, DMSO-d₆) δ 168.5, 150.1, 148.8, 130.0, 129.6, 124.9, 117.5, 32.0; MS (ES+) m/z 329.2 (M+1).

### Example 3

### Synthesis of biphenylene-1,4,5,8-tetrayltetrakis(methylene) tetracarbamimidothioate tetrahydrobromide

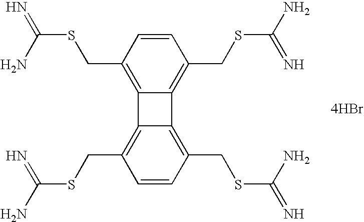

### A. Synthesis of 1,4,5,8-tetrakis(bromomethyl)biphenylene

To a stirred suspension of 1,4,5,8-tetramethylbiphenylene (0.048 g, 0.23 mmol) in carbon tetrachloride (10.0 mL) was added N-bromosuccinimide (0.17 g, 0.95 mmol) followed by the addition of benzoyl peroxide (0.006 g, 0.023 mmol). The mixture was stirred at reflux for 2 h, diluted with dichloromethane (40 mL) and washed with water. The organic layer was separated, dried over sodium sulfate and filtered. The filtrate was concentrated in vacuo and triturated with ethyl acetate. The pale yellow solid was collected by filtration and dried in air to afford 1,4,5,8-tetrakis(bromomethyl)biphenylene in 32% yield (0.04 g); ¹H NMR (300 MHz, DMSO-d₆) δ 6.90 (s, 4H), 4.56 (s, 8H).

### B. Synthesis of biphenylene-1,4,5,8-tetrayltetrakis(methylene) tetracarbamimidothioate tetrahydrobromide

To a solution of 1,4,5,8-tetrakisbromomethylbiphenylene (0.036 g, 0.068 mmol) in ethanol (4.0 mL) was added thiourea (0.022 g, 0.29 mmol). The mixture was maintained at 80° C. for 14 h and cooled to ambient temperature. Ethanol was removed to one third of the initial volume and hexane was added. The precipitation was collected by filtration, washed with ether and ethyl acetate and dried in air to afford biphenylene-1,4,5,8-tetrayltetrakis(methylene) tetracarbamimidothioate tetrahydrobromide in 91% yield (0.048 g): ¹H NMR (300 MHz, DMSO-d₆) δ 9.50-8.88 (m, 16H), 6.90 (s, 4H), 4.41 (s, 8H); MS (ES+) m/z 505.1 (M+1).

### Example 4

### Synthesis of (2,7-di-tert-butyl-9,9-dimethyl-9H-xanthene-4,5-diyl)bis(methylene) dicarbamimidothioate dihydrobromide

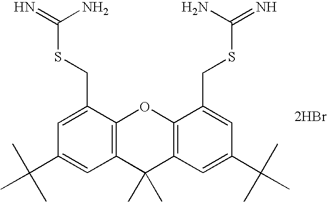

Thiourea (0.076 g, 1.00 mmol) was dissolved in 48% aqueous hydrobromic acid (1.5 mL). The mixture was stirred for 10 min and (2,7-di-tert-butyl-9,9-dimethyl-9H-xanthene-4,5-diyl)dimethanol (0.19 g, 0.50 mmol) was added in one portion. The mixture was stirred at 80° C. for 10 h and evaporated to dryness. The colorless solid obtained was washed with cold water and ether, and dried in vacuo to afford (2,7-di-tert-butyl-9,9-dimethyl-9H-xanthene-4,5-diyl)bis(methylene) dicarbamimidothioate dihydrobromide in 25% yield (0.062 g): ¹H NMR (300 MHz, DMSO-d₆) δ 9.19 (s, 4H), 9.05 (s, 4H), 7.48 (d, J=2.2 Hz, 2H), 7.37 (d, J=2.2 Hz, 2H), 4.60 (s, 4H), 1.58 (s, 6H), 1.26 (s, 18H); MS (ES+) m/z 499.1 (M+1).

### Example 5

### Synthesis of (3,6-difluorobiphenylene-1,8-diyl)bis(methylene) dicarbamimidothioate dihydrobromide

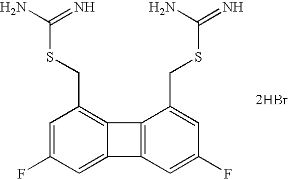

A. A stirred suspension of sodium borohydride (0.22 g, 6.00 mmol) in tetrahydrofuran (15 mL) was heated at 70° C. In one portion, dimethyl 3,6-difluorobiphenylene-1,8-dicarboxylate (0.15 g, 0.50 mmol) was added, and the resulting solution was heated for 1 h at reflux. The reaction mixture was treated with methanol (2 mL) and heating was continued for another 5 h, cooled to ambient temperature and concentrated in vacuo. The residue was dissolved in ethyl acetate (15 mL), washed with water (10 mL) and brine (10 mL), dried over sodium sulfate and concentrated in vacuo to afford (3,6-difluorobiphenylene-1,8-diyl)dimethanol as a yellow solid in 64% yield (0.078 g): ¹H NMR (300 MHz, DMSO-d₆) δ 6.60-6.49 (m, 4H), 5.26 (t, J=5.7 Hz, 2H), 4.31 (d, J=5.7 Hz, 4H).

B. A dry flask was charged with (3,6-difluorobiphenylene-1,8-diyl)dimethanol (0.080 g, 0.31 mmol), dichloromethane (10 mL) and diethyl ether (10 mL). The resulting solution was treated with phosphorus tribromide (0.10 mL, 0.93 mmol) and the solution was stirred for 20 h at ambient temperature under an nitrogen atmosphere. The reaction was quenched with water (20 mL) and the organic solvents removed in vacuo. The mixture was extracted with diethyl ether (3×10 mL) and the combined organic extract was washed with brine (10 mL), dried over sodium sulfate, filtered and concentrated in vacuo and the residue was purified by flash chromatography eluted with a gradient of 5 to 20% ethyl acetate in hexanes to afford 1,8-bis(bromomethyl)-3,6-difluorobiphenylene as a light yellow solid in 77% yield (0.09 g). ¹H NMR (300 MHz, CDCl₃) δ 6.45-6.40 (m, 4H), 4.33 (s, 4H).

C. A dry flask was charged with 1,8-bis(bromomethyl)-3,6-difluorobiphenylene (0.09 g, 0.24 mmol), thiourea (0.04 g, 0.48 mmol) and ethanol (10 mL). The reaction mixture was heated at reflux for 1.5 h, cooled to ambient temperature and filtered. The residue was washed with cold ethanol and dried to yield (3,6-difluorobiphenylene-1,8-diyl)bis(methylene) dicarbamimidothioate dihydrobromide as a light yellow powder in 63% yield (0.08 g): mp>250° C.; ¹H NMR (300 MHz, DMSO-d₆) δ 9.38-8.98 (m, 8H), 6.78-6.75 (m, 2H), 6.68-6.63 (m, 2H), 4.34 (s, 4H); ¹³C NMR (75 MHz, CDCl₃) δ 168.0, 162.2 (d, J_{C—F}=248.2 Hz), 149.5, 143.3, 126.7, 114.6 (d, J_{C—F}=24.3 Hz), 108.8 (d, J_{C—F}=28.0 Hz), 31.5; MS (ES+) m/z 365.2 (M+1).

### Biological Assays

Various techniques are known in the art for testing the activity of compounds of the invention. In order that the invention described herein may be more fully understood, the following biological assays are set forth. It should be understood that these examples are for illustrative purposes only and are not to be construed as limiting this invention in any manner.

### Biological Example 1

DMT1 Activity Assay

### In Vitro Assay

This example discloses various in vitro assay for testing and profiling test agents against DMT1 stably expressed in cells of either an endogenous or recombinant origin. These assays can use stable cell lines overexpressing DMT1 or intestinal cells and intestinal tissue expressing endogenous DMT1. DMT1 function could also be assessed in other cell types that express DMT1. Of greatest relevance would be the erythrocytes (e.g. K562 cells) or hepatocytes (e.g. HepG3).

DMT1 function can be assessed in a number of ways, including monitoring fluorescence changes of an iron fluorophore (e.g. calcein), monitoring uptake of radiolabelled iron (⁵⁵Fe or ⁵⁹Fe) (Picard et al., J. Biol. Chem., 2000, 275(46):35738-45 and Wetli et al., Chem. Biol. 2006 September; 13(9):965-72), or by assessing the current or transport of iron and other metals into the cells or tissues using standard electrophysiological techniques (Gunshin et al., Nature, 1997, 388(6641):482-8.).

Variations of these assays involve alterations of incubation times, the iron status of the cells and tissues (which may be modulated by chemical chelators or by harvesting from iron deficient animals), the metal cation detected and the pH of the reaction can generally be made by conventional techniques known to those skilled in the art.

### Biological Example 2

### In Vivo Assay for Treatment of Iron Disorders

This test measures the efficacy of compounds of the invention in blocking ferrous iron uptake in the duodenum in rats. The animals were rendered iron deficient by feeding an iron deficient diet for 3 weeks, which causes a marked decrease in serum iron and transferrin saturation. As a result of the iron deficiency, DMT1 expression in the duodenum is upregulated. The test animals were then given an oral bolus (or an “iron challenge”) of ferrous iron at 1 mg/kg resulting in a 20-fold increase in serum iron 1 hour post challenge. It was observed that when test animals were dosed with compound 1 hour prior to the iron challenge, there was a substantial reduction in the increase in serum iron level 1 hour post iron challenge. Compounds of the present invention were shown to be efficacious within a range of 30 mg/Kg and 0.1 mg/Kg.

Representative compounds of the invention, when tested in the above assay, demonstrated an IC₅₀ (nM) activity level as set forth below in Table 1 wherein “A” refers to an IC₅₀ activity level of from 1 nM to 10 nM, “B” refers to an IC₅₀ activity level from 10 nM to 100 nM, “C” refers to an IC₅₀ activity level from 100 nM to 1000 nM, and “D” refers to an IC₅₀ activity level equal to or greater than 1000 nM. The Example numbers provided in Table 1 correspond to the Examples herein:

TABLE 1

| | | IC₅₀ |
|---|---|---|
| Example | | Activity |
| No. | Compound Name | Level |
| 1 | dibenzo[b,d]thiophene-4,6-diylbis(methylene) | C |
| | dicarbamimidothioate | |
| 1.1 | (3,7-dibromodibenzo[b,d]thiophene-4,6-diyl)bis(methylene) | C |
| | dicarbamimidothioate | |
| 1.2 | (3-bromodibenzo[b,d]thiophene-4,6-diyl)bis(methylene) | C |
| | dicarbamimidothioate | |
| 1.3 | (3,7-dimethyldibenzo[b,d]furan-4,6-diyl)bis(methylene) | C |
| | dicarbamimidothioate | |
| 1.4 | (3,7-difluorodibenzo[b,d]furan-4,6-diyl)bis(methylene) | B |
| | dicarbamimidothioate | |
| 1.5 | (3,7-dichlorodibenzo[b,d]furan-4,6-diyl)bis(methylene) | C |
| | dicarbamimidothioate | |
| 1.6 | (2-fluorodibenzo[b,d]furan-4,6-diyl)bis(methylene) | B |
| | dicarbamimidothioate | |
| 1.7 | (2,8-dibromodibenzo[b,d]furan-4,6-diyl)bis(methylene) | B |
| | dicarbamimidothioate | |
| 1.8 | dibenzo[b,d]furan-4,6-diylbis(methylene) | B |
| | dicarbamimidothioate | |
| 1.9 | anthracene-1,8-diylbis(methylene) dicarbamimidothioate | C |
| 1.10 | (2-fluorodibenzo[b,d]thiophene-4,6-diyl)bis(methylene) | C |
| | dicarbamimidothioate | |
| 1.11 | (2-chloro-8-fluorodibenzo[b,d]thiophene-4,6- | B |
| | diyl)bis(methylene) dicarbamimidothioate | |
| 1.12 | phenoxathiine-4,6-diylbis(methylene) dicarbamimidothioate | C |
| 1.13 | (3,7-dibromodibenzo[b,d]furan-4,6-diyl)bis(methylene) | C |
| | dicarbamimidothioate | |
| 1.14 | dibenzo[b,d]thiophene-1,9-diylbis(methylene) | D |
| | dicarbamimidothioate | |
| 1.15 | (9-oxo-9H-fluorene-4,5-diyl)bis(methylene) | D |
| | dicarbamimidothioate | |
| 1.16 | (9-oxo-9H-xanthene-4,5-diyl)bis(methylene) | D |
| | dicarbamimidothioate | |
| 1.17 | (9-methylacridine-4,5-diyl)bis(methylene) | D |
| | dicarbamimidothioate | |
| 1.18 | acridine-4,5-diylbis(methylene) dicarbamimidothioate | C |
| 1.19 | (5,7-dihydrodibenzo[c,e]thiepine-1,11-diyl)bis(methylene) | D |
| | dicarbamimidothioate | |
| 2 | (9-oxo-9H-fluorene-1,8-diyl)bis(methylene) | C |
| | dicarbamimidothioate | |
| 2.1 | biphenylene-1,8-diylbis(methylene) dicarbamimidothioate | B |
| 3 | biphenylene-1,4,5,8-tetrayltetrakis(methylene) | C |
| | tetracarbamimidothioat | |
| 4 | (2,7-di-tert-butyl-9,9-dimethyl-9H-xanthene-4,5- | D |
| | diyl)bis(methylene) dicarbamimidothioate | |
| | dihydrobromide | |
| 5 | (3,6-difluorobiphenylene-1,8-diyl)bis(methylene) | B |
| | dicarbamimidothioate | |

A variation of this assay can be used for longer term studies. In this variation, animals are again rendered iron deficient by feeding of an iron deficient diet for 3 weeks. Then animals are switched back to an iron replete diet, while receiving a daily dose of either vehicle or a compound described herein. The vehicle animals recover their iron status, as measured by serum iron and other iron indicies, after 13 days. The drug treated animals, however, do not recover in this timeframe, as the compound is blocking the uptake of dietary iron. Other parameters that can be measured in both models include transferrin saturation, haemoglobin, hematocrit, liver iron and ferritin. More detailed assays can involve the use of radioactive metals as opposed to a bolus of ferrous iron. Multiple metals transported by DMT1 can be used to judge specificity of compound on cation uptake by DMT1, if any.

Genetic rat models of iron overload offers another format to show efficacy of DMT1 inhibitors in preventing further iron loading as development proceeds. These models are applicable to variety of human iron overload disorders such as hereditary hemochromatosis (Levy et al, Blood, 1999, 94:9-11, 1999), juvenile hemochromatosis (Huang et al, J. Clin. Invest., 2005 115:2187-2191), beta-2-microglobulin (de Sousa et al., Immun. Lett., 1994, 39:105-111, 1994), thalassemia (Ciavatta et al., Proc. Nat. Acad. Sci., 1995, 92: 9259-9263), hypotransferrinmia (Craven et. al., Proc. Nat. Acad. Sci., 1987, USA. 84(10):3457-61) and other hypochromic microcytic anemias.

In these models, the knock-out animals above are bred and treated with compound as they develop. Compound efficacy can be assessed by measuring reduced iron flux via the duodenum in a radioactive flux study or by monitoring whether chronic exposure to compounds cause a decrease in the amount of iron loading, as judged by serum iron, transferrin saturation, ferritin and liver iron. These models can be used with an iron bolus, or challenge, as above or iron may be absorbed from the diet. Where appropriate, a model of transfusional iron overload can be created in the rodent by transfusion of iron from another animal in order to exacerbate the iron overload is as seen clinically in the treatment of thalassemia.

All of the U.S. patents, U.S. patent application publications, U.S. patent applications, foreign patents, foreign patent applications and non-patent publications referred to in this specification are incorporated herein by reference in their entireties.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

What is claimed is:

1. A compound of formula (I): 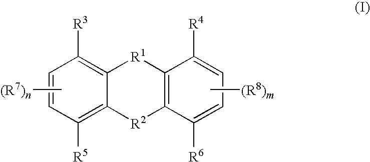
wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are different and are each independently selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
or R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³, or —R¹¹—N(R⁹)—C(═NR¹²)N(R¹²)R¹³;
R⁵ and R⁶ are different and are each independently selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
or R⁵ and R⁶ are the same and are selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R¹⁵, —R¹¹—C(O)OR¹⁴, —R¹¹—OC(O)R¹⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl, or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl;
as a stereoisomer, enantiomer, tautomer thereof or mixtures thereof;
or a pharmaceutically acceptable salt, solvate or prodrug thereof.

2. The compound of claim 1 wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³, or —R¹¹—N(R⁹)—C(═NR¹²)N(R¹²)R¹³;
R⁵ and R⁶ are the same and are selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)R¹⁴, —R¹¹—C(O)R¹⁴, —R¹¹—C(S)R¹⁵, —R¹¹—C(O)OR⁴, —R¹¹—OC(O)R¹⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl, or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl.

3. The compound of claim 2 wherein:
n and m are each independently 0, 1 or 2;
R¹ is —S—;
R² is a direct bond;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²) R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

4. The compound of claim 3 wherein:
n and m are each independently 0, 1 or 2;
R¹ is —S—;
R² is a direct bond;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

5. The compound of claim 4 wherein:
n and m are each independently 0, 1 or 2;
R¹ is —S—;
R² is a direct bond;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

6. The compound of claim 5 selected from the group consisting of: dibenzo[b,d]thiophene-4,6-diylbis(methylene) dicarbamimidothioate; (2-fluorodibenzo[b,d]thiophene-4,6-diyl)bis(methylene) dicarbamimidothioate; (3,7-dibromodibenzo[b,d]thiophene-4,6-diyl)bis(methylene) dicarbamimidothioate; (2-chloro-8-fluorodibenzo[b,d]thiophene-4,6-diyl)bis(methylene) dicarbamimidothioate; and (3-bromodibenzo[b,d]thiophene-4,6-diyl)bis(methylene) dicarbamimidothioate.

7. The compound of claim 2 wherein:
n and m are each independently 0, 1 or 2;
R¹ is a direct bond;
R² is —S—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

8. The compound of claim 7 which is dibenzo[b,d]thiophene-1,9-diylbis(methylene) dicarbamimidothioate.

9. The compound of claim 2 wherein:
n and m are each independently 0, 1 or 2;
R¹ is —O—;
R² is a direct bond or —C(O)—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

10. The compound of claim 9 wherein:
n and m are each independently 0, 1 or 2;
R¹ is —O—;
R² is a direct bond or —C(O)—;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

11. The compound of claim 10 wherein:
n and m are each independently 0, 1 or 2;
R¹ is —O—;
R² is a direct bond or —C(O)—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

12. The compound of claim 11 selected from the group consisting of: (9-oxo-9H-xanthene-4,5-diyl)bis(methylene) dicarbamimidothioate; dibenzo[b,d]furan-4,6-diylbis(methylene) dicarbamimidothioate; (3,7-dimethyldibenzo[b,d]furan-4,6-diyl)bis(methylene) dicarbamimidothioate; (3,7-dichloroldibenzo[b,d]furan-4,6-diyl)bis(methylene) dicarbamimidothioate; (3,7-dibromodibenzo[b,d]furan-4,6-diyl)bis(methylene) dicarbamimidothioate; (2-fluorodibenzo[b,d]furan-4,6-diyl)bis(methylene) dicarbamimidothioate; and (2,8-dibromodibenzo[b,d]furan-4,6-diyl)bis(methylene) dicarbamimidothioate.

13. The compound of claim 2 wherein:
n and m are each independently 0, 1 or 2;
R¹ is a direct bond;
R² is a direct bond;
R³ and R⁴ are the same and are selected from —R¹¹—S—C(═NR¹²)N(R¹²)R¹³, —R¹¹—O—C(═NR¹²)N(R²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

14. The compound of claim 13 wherein:
n and m are each independently 0, 1 or 2;
R¹ is a direct bond;
R² is a direct bond;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

15. The compound of claim 14 wherein:
n and m are each independently 0, 1 or 2;
R¹ is a direct bond;
R² is a direct bond;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

16. The compound of claim 15 selected from the group consisting of: biphenylene-1,8-diylbis(methylene) dicarbamimidothioate; and (3,6-difluorobiphenylene-1,8-diyl)bis(methylene) dicarbamimidothioate.

17. The compound of claim 14 wherein:
n and m are each independently 0, 1 or 2;
R¹ is a direct bond;
R² is a direct bond;
R³ and R⁴ are both —R¹¹—S—C(═NR¹²)N(R¹²)R¹³;
R⁵ and R⁶ are both —R¹¹—S—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

18. The compound of claim 17 which is biphenylene-1,4,5,8-tetrayltetrakis(methylene) tetracarbamimidothioate.

19. The compound of claim 2 wherein:
n and m are each independently 0, 1 or 2;
R¹ is —C(O)—;
R² is a direct bond;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

20. The compound of claim 19 wherein:
n and m are each independently 0, 1 or 2;
R¹ is —C(O)—;
R² is a direct bond;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

21. The compound of claim 20 wherein:
n and m are each independently 0, 1 or 2;
R¹ is —C(O)—;
R² is a direct bond;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

22. The compound of claim 21 which is 2-(8-carbamimidoylsulfanylmethyl-9-oxo-9H-fluoren-1-ylmethyl)-isothiourea.

23. The compound of claim 2 wherein:
n and m are each independently 0, 1 or 2;
R¹ is a direct bond;
R² is —C(O)—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹″-OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

24. The compound of claim 23 which is (9-oxo-9H-fluorene-4,5-diyl)bis(methylene) dicarbamimidothioate.

25. The compound of claim 2 wherein:
n and m are each independently 0, 1 or 2;
R¹ is —O—;
R² is —C(R⁹)₂—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

26. The compound of claim 25 wherein:
n and m are each independently 0, 1 or 2;
R¹ is —O—;
R² is —C(R⁹)₂—;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

27. The compound of claim 26 wherein:
n and m are each independently 0, 1 or 2;
R¹ is —O—;
R² is —C(R⁹)₂—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

28. The compound of claim 27 which is 2-(2,7-di-tert-butyl-5-carbamimidoylsulfanylmethyl-9,9-dimethyl-9H-xanthen-4-ylmethyl)-isothiourea.

29. The compound of claim 2 wherein:
n and m are each independently 0, 1 or 2;
R¹ is —O—;
R² is —S—;
R³ and R⁴ are the same and are selected from —R¹¹—S—C(═NR¹²)N(R²)R¹³, —R¹¹—O—C(═NR¹²)N(R¹²)R³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR²)N(R²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
each R⁷ and R³ is independently selected from the group consisting of —R¹″-OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

30. The compound of claim 29 wherein:
n and m are each independently 0, 1 or 2;
R¹ is —O—;
R² is —S—;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

31. The compound of claim 30 wherein:
n and m are each independently 0, 1 or 2;
R¹ is —O—;
R² is —S—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

32. The compound of claim 31 which is phenoxathiine-4,6-diylbis(methylene) dicarbamimidothioate.

33. The compound of claim 2 wherein:
n and m are each independently 0, 1 or 2;
R¹ is a direct bond;
R² is —CH₂—S—CH₂—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, —R¹¹—S—C(═NR¹²)N(R¹²)R¹³, —R¹¹—O—C(═NR¹²)N(R¹²)R¹³, R¹¹—C(═NR¹²)N(R¹²)R¹³ or —R¹¹—N(R⁹)—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R¹¹ is independently a direct bond or a straight or branched alkylene chain; and
each R¹² and R¹³ is independently hydrogen, alkyl or —OR⁹.

34. The compound of claim 33 wherein:
n and m are each independently 0, 1 or 2;
R¹ is a direct bond;
R² is —CH₂—S—CH₂—;
R³ and R⁴ are both —R¹¹—S—C(═NR¹²)N(R¹²)R¹³;
R⁵ and R⁶ are the same and are selected from hydrogen or —R¹¹—S—C(═NR¹²)N(R¹²)R¹³;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

35. The compound of claim 34 wherein:
n and m are each independently 0, 1 or 2;
R¹ is a direct bond;
R² is —CH₂—S—CH₂—;
R³ and R⁴ are both —R¹¹—S—C(═NR¹²)N(R¹²)R¹³;
R⁵ and R⁶ are both hydrogen;
each R⁷ and R⁸ is independently selected from the group consisting of —R¹¹—OR⁹, alkyl, halo and haloalkyl;
each R⁹ is hydrogen, alkyl, haloalkyl, alkoxyalkyl, optionally substituted aryl or optionally substituted aralkyl;
each R¹¹ is independently a direct bond or a straight or branched alkylene chain; and
each R¹² and R¹³ is independently hydrogen, alkyl or —OR⁹.

36. The compound of claim 35 which is (5,7-dihydrodibenzo[c,e]thiepine-1,11-diyl)bis(methylene) dicarbamimidothioate.

37. The compound of claim 1 wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are different and are each independently selected from —R¹¹—S—C(═NR¹²)N(R¹²)R¹³, —R¹¹—O—C(═NR¹²)N(R²)R¹³, —R¹¹—C(═NR¹²)N(R¹²)R¹³ or —R¹¹—N(R⁹)—C(═NR¹²)N(R¹²)R¹³;
R⁵ and R⁶ are different and are each independently selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R¹⁵, —R¹¹—C(O)OR¹⁴, —R¹¹—OC(O)R¹⁴, —R¹¹—C(S)OR¹⁴, —R¹¹—C(O)N(R¹⁴)₂—R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl.

38. The compound of claim 1 wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are different and are each independently selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are the same and are selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R¹⁵, —R¹¹—C(O)OR¹⁴, —R¹¹—OC(O)R¹⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)R¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl.

39. The compound of claim 1 wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are different and are each independently selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³—N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R⁵, —R¹¹—C(O)OR¹⁴, —R¹¹—C(O)R¹⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)N(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl.

40. A pharmaceutical composition comprising a pharmaceutically acceptable excipient and a compound of formula (I): 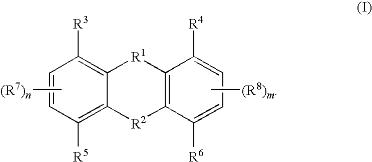
wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are different and are each independently selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³, or —R¹¹—N(R⁹)—C(═NR¹²)N(R¹²)R¹³;
or R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³, or —R¹¹—N(R⁹)—C(═NR¹²)N(R¹²)R¹³;
R⁵ and R⁶ are different and are each independently selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)R¹⁵, —S(O)ₜOR¹⁵, —S(O)ₚR¹⁴, or —S(O)ₜN(R¹⁴)₂, wherein each t is independently 1 or 2 and each p is 0, 1 or 2;
or R⁵ and R⁶ are the same and are selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R¹⁵, —R¹¹—C(O)OR¹⁴, —R¹¹—OC(O)R¹⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl, or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl;
as a stereoisomer, enantiomer, tautomer thereof or mixtures thereof;
or a pharmaceutically acceptable salt, solvate or prodrug thereof.

41. A method of treating an iron disorder in a mammal, wherein the method comprises administering to the mammal a therapeutically effective amount of a compound of formula (I): 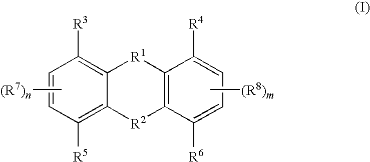
wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are different and are each independently selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
or R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R³, or —R¹¹—N(R⁹)—C(═NR¹²)N(R¹²)R¹³;
R⁵ and R⁶ are different and are each independently selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
or R⁵ and R⁶ are the same and are selected from hydrogen, alkyl, halo, haloalkyl, —R¹¹—CN, —R¹¹—NO₂, —R¹¹—N(R⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R¹⁵, —R¹¹—C(O)OR¹⁴, —R¹¹—OC(O)R¹⁴, —R¹¹—C(S)OR¹⁴, —R¹¹—C(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl, or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl;
as a stereoisomer, enantiomer, tautomer thereof or mixtures thereof;
or a pharmaceutically acceptable salt, solvate or prodrug thereof.

42. A method of treating a disease or condition associated with an iron disorder in a mammal, wherein the method comprises administering to the mammal in need thereof a therapeutically effective amount of a compound of formula (I): 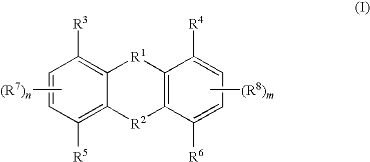
wherein:
n and m are each independently 0, 1 or 2;
R¹ and R² are each independently a direct bond, —C(R⁹)₂—, —S—, —O—, —C(O)—, —N(R⁹)— or —CH₂—R¹⁰—CH₂—;
R³ and R⁴ are different and are each independently selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³, or —R¹¹—N(R⁹)—C(═NR¹²)N(R¹²)R¹³;
or R³ and R⁴ are the same and are selected from —R¹¹—S—C(═NR¹²)N(R¹²)R¹³, —R¹¹—O—C(═NR¹²)N(R¹²)R¹³, —R¹¹—C(═NR¹²)N(R¹²)R¹³ or —R¹¹—N(R⁹)—C(═NR¹²)N(R¹²)R¹³;
R⁵ and R⁶ are different and are each independently selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹″-N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
or R⁵ and R⁶ are the same and are selected from hydrogen, alkyl, halo, haloalkyl, —R¹¹—CN, —R¹¹—NO₂, —R¹¹—N(R¹⁴)₂, —R¹¹—C(O)OR¹⁴, —R¹¹—C(O)N(R¹⁴)₂, —R¹¹—S—C(═NR¹²)N(R¹²)R¹³, —R¹¹—O—C(═NR¹²)N(R¹²)R¹³, —R¹¹—C(═NR¹²)N(R¹²)R¹³, —R¹¹—N(R⁹)—C(═NR¹²)N(R¹²)R¹³, —N(R¹⁴)S(O)ₜR¹⁵, —S(O)ₜOR¹⁵, —S(O)ₚR¹⁴, or —S(O)ₜN(R¹⁴)₂, wherein each t is independently 1 or 2 and each p is 0, 1 or 2;
each R⁷ and R⁸ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R¹¹—CN, —R¹¹—NO₂, —R¹¹—OR⁹, —R⁵—OS(O)₂R¹⁵, —R¹¹—N(R¹⁴)₂, —R¹¹—S(O)ₚR¹⁴, —R¹¹—C(O)R¹⁴, —R¹¹—C(S)R¹⁵, —R¹¹—C(O)OR¹⁴, —R¹¹—C(O)R¹⁴, —R¹¹—C(S)OR¹⁴, R¹¹—(O)N(R¹⁴)₂, —R¹¹—C(S)N(R¹⁴)₂, —N═C(R¹⁵)₂, —R¹¹—N(R¹⁴)C(O)R¹⁵, —R¹¹—N(R¹⁴)C(S)R¹⁵, —R¹¹—N(R¹⁴)C(O)OR¹⁴, —R¹¹—N(R¹⁴)C(S)OR¹⁴, —R¹¹—N(R¹⁴)C(O)N(R¹⁴)₂, —R¹¹—N(R¹⁴)C(S)N(R¹⁴)₂, —R¹¹—N(R¹⁴)S(O)ₜR¹⁴, —R¹¹—N(R¹⁴)S(O)ₜN(R¹⁴)₂, —R¹¹—S(O)ₜN(R¹⁴)₂, —R¹¹—N(R¹⁴)C(═NR¹⁴)N(R¹⁴)₂, and —R¹¹—N(R¹⁴)C(N═C(R¹⁴)₂)N(R¹⁴)₂, wherein each p is independently 0, 1, or 2 and each t is independently 1 or 2;
each R⁹ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
R¹⁰ is —C(R⁹)₂—, —S—, —O— or —N(R⁹)—;
each R¹¹ is independently a direct bond or a straight or branched alkylene chain;
each R¹² and R¹³ is independently hydrogen, alkyl, or —OR⁹;
each R¹⁴ is independently hydrogen, alkyl, optionally substituted aryl, optionally substituted aralkyl, optionally substituted heteroaryl or optionally substituted heteroaryl; and
each R¹⁵ is alkyl;
as a stereoisomer, enantiomer, tautomer thereof or mixtures thereof;
or a pharmaceutically acceptable salt, solvate or prodrug thereof.

43. A compound of formula (II): 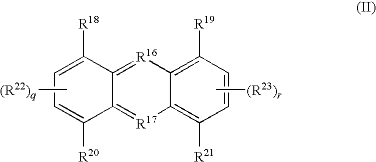
wherein:
q and r are each independently 0, 1 or 2;
R¹⁶ and R¹⁷ are each independently ═C(R²⁴)— or ═N—;
R¹⁸ and R¹⁹ are different and are each independently selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
or R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are different and are each independently selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
or R²⁰ and R²¹ are the same and are selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)₂, —R²⁵—S(O)R²⁸, —R²⁵—C(O)R²⁸, —R²⁵—C(S)R²⁹, R²⁵—C(O)R²⁸, R²⁵—OC(O)R²⁸,—R²⁵—C(S)OR²⁸, —R²⁵—C(O)N(R²)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl;
as a stereoisomer, enantiomer, tautomer thereof or mixtures thereof;
or a pharmaceutically acceptable salt, solvate or prodrug thereof.

44. The compound of claim 43 wherein:
q and r are each independently 0, 1 or 2;
R¹⁶ and R¹⁷ are each independently ═C(R²⁴)— or ═N—;
R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are the same and are selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(R²⁶)N(R²⁶)R²⁷, R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, R²⁵, —R²⁴, —R²⁵—OS(O)₂R²⁹, —R²⁵—N(R²⁸)₂, —R²⁵—S(O)ₚR²⁸, —R²⁵—C(O)R²⁸, —R²⁵—C(S)R²⁹, —R²⁵—C(O)OR²⁸, —R²⁵—OC(O)R²⁸, —R²⁵—C(S)OR²⁸, —R²⁵—C(O)N(R²⁸) —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl.

45. The compound of claim 44 wherein:
q and r are each independently 0, 1 or 2;
R¹⁶ and R¹⁷ are each ═C(R²⁴)—;
R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
R²⁰ and R²¹ are the same and are selected from hydrogen, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
each R²² and R²³ is independently selected from the group consisting of —R²⁵—OR²⁴, alkyl, halo and haloalkyl;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain; and
each R²⁶ and R²⁷ is independently hydrogen, alkyl or —OR²⁴.

46. The compound of claim 45 wherein:
q and r are each independently 0, 1 or 2;
R¹⁶ and R¹⁷ are each ═C(R²⁴)—;
R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
R²⁰ and R²¹ are the same and are selected from hydrogen, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
each R²² and R²³ is independently selected from the group consisting of —R²⁵—OR²⁴, alkyl, halo and haloalkyl;
each R²⁴ is hydrogen, alkyl, haloalkyl, alkoxyalkyl, optionally substituted aryl or optionally substituted aralkyl;
each R²⁵ is independently a direct bond or a straight or branched alkylene chain; and
each R²⁶ and R²⁷ is independently hydrogen, alkyl or —OR²⁴.

47. The compound of claim 46 wherein:
q and r are each independently 0, 1 or 2;
R¹⁶ and R¹⁷ are each ═C(R²⁴)—;
R¹⁸ and R¹⁹ are both —R²⁵—S—C(═NR²⁶)N(R²⁶)R²⁷;
R²⁰ and R²¹ are both hydrogen;
each R²² and R²³ is independently selected from the group consisting of —R²⁵—OR²⁴, alkyl, halo and haloalkyl;
each R²⁴ is hydrogen, alkyl, haloalkyl, alkoxyalkyl, optionally substituted aryl or optionally substituted aralkyl;
each R²⁵ is independently a direct bond or a straight or branched alkylene chain; and
each R²⁶ and R²⁷ is independently hydrogen, alkyl or —OR²⁴.

48. The compound of claim 47 which is anthracene-1,8-diylbis(methylene) dicarbamimidothioate.

49. The compound of claim 44 wherein:
q and r are each independently 0, 1 or 2;
R¹⁶ is ═N—;
R¹⁷ is ═C(R²⁴)—;
R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
R²⁰ and R²¹ are the same and are selected from hydrogen, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
each R²² and R²³ is independently selected from the group consisting of —R²⁵—OR²⁴, alkyl, halo and haloalkyl;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain; and
each R²⁶ and R²⁷ is independently hydrogen, alkyl or —OR²⁴.

50. The compound of claim 49 wherein:
q and r are each independently 0, 1 or 2;
R¹⁶ is ═N—;
R¹⁷ is ═C(R²⁴)—;
R¹⁸ and R¹⁹ are the same and are selected from —R²⁵—S—C(═NR²⁶)N(R²⁶)R²⁷, —R²⁵—C(═NR²⁶)N(R²⁶)R²⁷, —R²⁵—C(═NR²⁶)N(R²⁶)R²⁷ or —R²⁵—N(R²⁴)—C(═NR²⁸)N(R²⁶)R²⁷;
R²⁰ and R²¹ are the same and are selected from hydrogen, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R²⁴)—C(═NR²⁶)N(R²⁶)R²⁷;
each R²² and R²³ is independently selected from the group consisting of —R²⁵—OR²⁴, alkyl, halo and haloalkyl;
each R²⁴ is hydrogen, alkyl, haloalkyl, alkoxyalkyl, optionally substituted aryl or optionally substituted aralkyl;
each R²⁵ is independently a direct bond or a straight or branched alkylene chain; and
each R²⁶ and R²⁷ is independently hydrogen, alkyl or —OR²⁴.

51. The compound of claim 50 wherein:
q and r are each independently 0, 1 or 2;
R¹⁶ is ═N—;
R¹⁷ is ═C(R²⁴)—;
R¹⁸ and R¹⁹ are both —R²⁵—S—C(═NR²⁶)N(R²⁶)R²⁷;
R²⁰ and R²¹ are both hydrogen;
each R²² and R²³ is independently selected from the group consisting of —R²⁵—OR²⁴, alkyl, halo and haloalkyl;
each R²⁴ is hydrogen, alkyl, haloalkyl, alkoxyalkyl, optionally substituted aryl or optionally substituted aralkyl;
each R²⁵ is independently a direct bond or a straight or branched alkylene chain; and
each R²⁶ and R²⁷ is independently hydrogen, alkyl or —OR²⁴.

52. The compound of claim 51 selected from the group consisting of: acridine-4,5-diylbis(methylene) dicarbamimidothioate; and (9-methylacridine-4,5-diyl)bis(methylene) dicarbamimidothioate.

53. The compound of claim 43 wherein:
q and r are each independently 0, 1 or 2;
R¹⁶ and R¹⁷ are each independently ═C(R²⁴)— or ═N—;
R¹⁸ and R¹⁹ are different and are each independently selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are different and are each independently selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, R²⁵, —R²⁴, —R²⁵—OS(O)₂R²⁹, —R²⁵—N(R²⁸)₂, —R²⁵—S(O)R²⁸, —R²⁵—C(O)R²⁸, —R²⁵—C(S)R²⁹, —R²⁵—C(O)OR²⁸, —R²⁵—OC(O)R²⁸, —R²⁵—C(S)OR²⁸, —R²⁵—C(O)N(R²⁸)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁶ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl.

54. The compound of claim 43 wherein:
q and r are each independently 0, 1 or 2;
R¹⁶ and R¹⁷ are each independently ═C(R²⁴)— or ═N—;
R¹⁸ and R¹⁹ are different and are each independently selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are the same and are selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)₂, —R²⁵—S(O)R²⁸, —R²⁵—C(O)R²⁸, —R²⁵—C(S)R²⁹, —R²⁵—C(O)OR²⁸, —R²⁵—OC(O)R²⁸, —R²⁵—C(S)OR²⁸, —R²⁵—C(O)N(R²⁸)₂, R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl.

55. The compound of claim 43 wherein:
q and r are each independently 0, 1 or 2;
R¹⁶ and R¹⁷ are each independently ═C(R²⁴)— or ═N—;
R¹⁸ and R¹⁹ are the same and are selected from —R²⁵—S—C(═NR²⁶)N(R²⁶)R²⁷, —R²⁵—O—C(NR²⁶)N(R²⁶)R²⁷, —R²⁵—C(NR²⁶)N(R²⁶)R²⁷ or —R²⁵—N(R⁹)—C(═NR²⁶)N(R²⁶)R²⁷;
R²⁰ and R²¹ are different and are each independently selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, R²⁵—S—C(═NR²⁶)N(R²⁶)R²⁷, —R²⁵—O—C(═NR²⁶)N(R²⁶)R —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)₂, —R²⁵—S(O)ₚR²⁸, —R²⁵—C(O)R²⁸, —R²⁵—C(S)R²⁹, R²⁵—C(O)OR²⁸, R²⁵—OC(O)R²⁸, R²⁵—C(S)OR²⁸, —R²⁵—C(O)N(R²⁸)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl.

56. A pharmaceutical composition comprising a pharmaceutically acceptable excipient and a compound of formula (II): 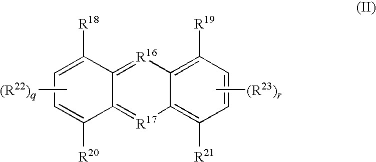
wherein:
q and r are each independently 0, 1 or 2;
R¹⁶ and R¹⁷ are each independently ═C(R²⁴)— or ═N—;
R¹⁸ and R¹⁹ are different and are each independently selected from —R²⁵—S—C(NR²⁶)N(R²⁶)R²⁷, —R²⁵—O—C(═NR^{R2}26)R²⁷, —R²⁵—C(═NR²⁶)N(R²⁶)R²⁷ or —R²⁵—N(R⁹)—C(═NR²⁶)N(R²⁶)R²⁷;
or R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are different and are each independently selected from hydrogen, alkyl, halo, haloalkyl, —R²⁵—CN, —R²⁵—NO₂, —R²⁵—N(R²⁸)₂, —R²⁵—C(O)OR²⁸, R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
or R²⁰ and R²¹ are the same and are selected from hydrogen, alkyl, halo, haloalkyl, —R²⁵—CN, —R²⁵—NO₂, —R²⁵—N(R²⁸)₂, —R²⁵—C(O)OR², R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹—S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, R²⁵—OR²⁴, —R²⁵—OS(O)₂R²⁹, —R²⁵—N(R²⁸)₂, —R²⁵—S(O)ₚR²⁸, —R²⁵—C(O)R²⁸, —R²⁵—C(S)R²⁹, —R²⁵—C(O)OR²⁸, —R²⁵—OC(O)R²⁸, —R²⁵—C(S)R²⁸, —R²⁵—C(O)N(R²⁸)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(s)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl;
as a stereoisomer, enantiomer, tautomer thereof or mixtures thereof;
or a pharmaceutically acceptable salt, solvate or prodrug thereof.

57. A method of treating an iron disorder in a mammal, wherein the method comprises administering to the mammal a therapeutically effective amount of a compound of formula (II): 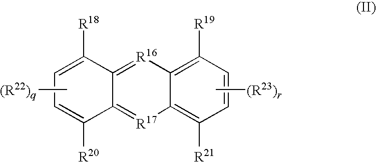
wherein:
q and r are each independently 0, 1 or 2;
R¹⁶ and R¹⁷ are each independently ═C(R²⁴)— or ═N—;
R¹⁸ and R¹⁹ are different and are each independently selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
or R¹⁸ and R¹⁹ are the same and are selected from —R²⁵—S—C(═NR²⁶)N(R²⁶)R²⁷, —R²⁵—O—C(═NR²⁶)N(R²⁶)R²⁷, —R²⁵—C(═NR²)N(R²⁶)R²⁷ or —R²⁵—N(R⁹)—C(═NR²⁶)N(R²⁶)R²⁷;
R²⁰ and R²¹ are different and are each independently selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
or R²⁰ and R²¹ are the same and are selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)ₐ, —R²⁵—S(O)ₚR²⁸, —R²⁵—C(O)R²⁸, —R²⁵—C(S)R²⁹, —R²⁵—C(O)OR²⁸, —R²⁵—OC(O)R²⁸, —R²⁵—C(S)OR²⁸, —R²⁵—C(O)N(R²⁸)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²⁸)₂, —R²⁵—N(R²⁸)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl;
as a stereoisomer, enantiomer, tautomer thereof or mixtures thereof;
or a pharmaceutically acceptable salt, solvate or prodrug thereof.

58. A method of treating a disease or condition associated with an iron disorder in a mammal, wherein the method comprises administering to the mammal in need thereof a therapeutically effective amount of a compound of formula (II): 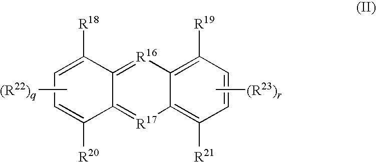
wherein:
q and r are each independently 0, 1 or 2;
R¹⁶ and R¹⁷ are each independently ═C(R²⁴)— or ═N—;
R¹⁸ and R¹⁹ are different and are each independently selected from —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
or R¹⁸ and R¹⁹ are the same and are selected from —R²⁵—S—C(═NR²⁶)N(R²⁶)R²⁷, —R²⁵—OC(═NR²⁶)N(R²⁶)R²⁷, —R²⁵—C(═NR²⁶)N(R²⁶)R²⁷ or —R²⁵—N(R⁹)—C(═NR²⁶)N(R²⁶)R²⁷;
R²⁰ and R²¹ are different and are each independently selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²⁶)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
or R²⁰ and R²¹ are the same and are selected from hydrogen, alkyl, halo, haloalkyl, —R²⁵—CN, —R²⁵—NO₂, —R²⁵—N(R²⁸)₂, —R²⁵—C(O)OR²⁸, —R²⁵—C(O)N(R²⁸)₂, —R²⁵—S—C(═NR²⁶)N(R²⁶)R²⁷, —R²⁵—O—C(═NR²⁶)N(R²⁶)R²⁷, —R²⁵—C(═NR²⁶)N(R²)R²⁷, —R²⁵—N(R⁹)—C(═NR²⁶)N(R²⁶)R²⁷, —N(R²⁸)S(O)ₜR²⁹, —S(O)ₜOR²⁹, —S(O)ₚR²⁸, or —S(O)ₜN(R²⁸)₂, wherein each t is independently 1 or 2 and each p is 0, 1 or 2;
each R²² and R²³ is independently selected from the group consisting of alkyl, alkenyl, alkynyl, halo, haloalkyl, haloalkenyl, haloalkoxy, optionally substituted cycloalkyl, optionally substituted cycloalkylalkyl, optionally substituted aryl, optionally substituted aralkyl, optionally substituted aralkenyl, optionally substituted heterocyclyl, optionally substituted heterocyclylalkyl, optionally substituted heteroaryl, optionally substituted heteroarylalkyl, —R²⁵—CN, —R²⁵—NO₂, —R²⁵—OR²⁴, —R²⁵—OS(O)₂R²⁹, —R²⁵—N(R²⁸)₂, —R²⁵—S(O)R²⁸R²⁵—C(O)R²⁸, —R²⁵— C(S)R²⁹, —R²⁵—C(O)R²⁸, —R²⁵—OC(O)R²⁸, —R²⁵—C(S)OR²⁸, —R²⁵—C(O)N(R²⁸)₂, —R²⁵—C(S)N(R²⁸)₂, —N═C(R²⁹)₂, —R²⁵—N(R²⁸)C(O)R²⁹, —R²⁵—N(R²⁸)C(S)R²⁹, —R²⁵—N(R²⁸)C(O)OR²⁸, —R²⁵—N(R²⁸)C(S)OR²⁸, —R²⁵—N(R²⁸)C(O)N(R²⁸)₂, —R²⁵—N(R²⁸)C(S)N(R²)₂, —R²⁵—N(R²″)S(O)ₜR²⁸, —R²⁵—N(R²⁸)S(O)ₜN(R²⁸)₂, —R²⁵—S(O)ₜN(R²⁸)₂, —R²⁵—N(R²⁸)C(═NR²⁸)N(R²⁸)₂, and —R²⁵—N(R²⁸)C(N═C(R²⁸)₂)N(R²⁸)₂, wherein each p is independently 0, 1, or 2 and each t is independently 1 or 2;
each R²⁴ is hydrogen, alkyl, alkenyl, alkynyl, haloalkyl, alkoxyalkyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl or optionally substituted heteroarylalkyl;
each R²⁵ is independently a direct bond or a straight or branched alkylene chain;
each R²⁶ and R²⁷ is independently hydrogen, alkyl or —OR²⁴;
each R²⁸ is independently hydrogen, alkyl, optionally substituted aryl, optionally substituted aralkyl, optionally substituted heteroaryl or optionally substituted heteroaryl; and
each R²⁹ is alkyl;
as a stereoisomer, enantiomer, tautomer thereof or mixtures thereof;
or a pharmaceutically acceptable salt, solvate or prodrug thereof.
